SEQUENCE LISTING (49 sequences)

SEQ ID NO: 1 - DNA, 2885 bp, synthetic construct
  misc_feature 1..2885: note = Synthetic: gRNA plasmid sequence
  source 1..2885: mol_type = other DNA
gaattctaaagatctttgacagctagctcagtcctaggtataatactagttctggcgcagttgatatgtagttttagagctagaaatagcaagttaaaataaggctagtccgttatcaacttgaaaaagtggcaccgagtcggtgctttttttgaagcttgggcccgaacaaaaactcatctcagaagaggatctgaatagcgccgtcgaccatcatcatcatcatcattgagtttaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaactggatccttactcgagtctagactgcaggcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgacacatttccccgaaaagtgccacctgacgtctaagaaaccattattatcatgacattaacctataaaaataggcgtatcacgaggcagaatttcagataaaaaaaatccttagctttcgctaaggatgatttctg

SEQ ID NO: 2 - DNA, 2068 bp, synthetic construct
  misc_feature 1..2068: note = Synthetic: repair DNA sequence
  source 1..2068: mol_type = other DNA
acgaattcgagctcggtacccggggatcctatatgtgccggacgccgaccgtgccgccccagctgattgagcagatgctgcaccggcccgtcaggggggcagccgtaccctgacggcacgacgctgcggaactgatagcgcccctgttcatcggtaatgatggtacgacgcaggttaaacgcactctgacttttatcgaagaacgagtaattaccgagggtattggcgtgccagacctcaacgctggcgccgggaagcggttgaccctcttcgttccataccgtgccctgcatcaacagcacttcgccctggttatcttcagaaccatcgtccagccgcgcccaggcctgacagactggcgccccggccacatagagaggaccttcgatggtgcgcggtgtcccaccgtgcagccccgccctcgcttcagcctcatccgcccggatatccataaaacgctccagtccggttcccgcagccagcagccccatctcctgagcgataccggtatcggccagatattccagccctttccagatctcagtttgctgcaggcccagatcctcaatcgctttaaacagatcgctcaggagacgaacggtgacctgctgaatacgtgggttcaccgtgcctttcgcggttttgacgataaagcagttaaccaggttgtcgatctctttgctgttcatattgacgctccatttcaggtacagctcaggatgagaaagtatttaacgatcgtccacacgcaccgccgaagggtggcggcacagcggcactatctgcatcgtcataaagggataaagcggcagctcactcagcagctggtgcaactcctcaacgctatcgacgtcaaatacgctgacgttggcgtaatgcccggcaatgcgccagatgtgtcgccacttgccgcgacgctgaagcgcctgcgagtaggctttttcccgtgccttaagttcgctggcgacattttccgccatcgtctgcggcagattcacatccatagttacgtgaaatagcatggtgaaatttcctcctacgctatagaatcccgccgatcggggatagccaaatcctgctgaaaatgattgacccgctgtatgagaatgcgcagatcgcgcctgttcgccccagctcacataacttgcgataaaaacgtcttttagtgccaatgacacttttaggttattttttcgccggtgaagtcagcaccctgatgactcaaactcaggaggagcgagatgatcgacaaaagtctgtttttggccgatgaagccgtggctgacattcacgatggtgccacgctgatgattggcggatttggcccggcaggacagccctatgcgctgctggacgcactgattcggcggcggccacagaacctcacgctggtcagcaacaacgctggcaacgctgattcgggtctggcgctactgctcaaagcggggtgcgtgcgcaagatgatctgttcctttccgcgccagagtgactcatgggtatttgacgacctctaccgccgcggagagattgagctggagctggtgccgcagggcaatcttgtggcgcgtattcaggcaggtggcagcgggctgggggccatttttaccccaacgggtttcggtactgaactggctgcgggcaaagagacgcgcgaaattgaggggcggcagtacgtgctggaactggcgctgaaggcagactttgccctgatcaaagcacagtgcggtgatcgctggggtaacctggtctatagcaaagcggcacgcaacttcaacccgattatggcgatggctgcccgctgcaccatcgctgaagtctcccgccacgtcgcgctgggcaagctggacccggaggcggtggtgactcccggtatttttgtgcagcgcgtggtctgttcagccaatctcagccccgcaacgatcgcctcaggagcgtgacacaatgcaactcatccgcctcacgcataaccagctggcgcagcgcatcgcccgcgatatcccggacggagcctacgtcaatcttggcatcggcctgccaacctgcaggcatgcaagcttggcactggccg

SEQ ID NO: 3 - DNA, 1110 bp, Erwinia aphidicola
  misc_feature 1..1110: note = LJJL01muconate cycloisomerase gene sequence (Gene ID: 2752379084)
  source 1..1110: mol_type = genomic DNA
ttaacggcggcgcagcgccgcgatcctgtcgcgatcgagggcaatgcccagcccgggcccggtcgggacctgcagcataaaatcacgataaactagcggttcactgagaatatcttcggtcagcagcagcgggccaaacagctcagtgccgaaactcaaatcgttgaaggtggcgcagagatgcgccgtggcggccgtgcctacggcgccttccagcatggtgcccccgtaaagcgcgatatctgccagctgggcaatatcggcaacccggcgcgcctgcgtcagcccgccggactgggtaatttttatcgagaacacgtcggcggcagcgtggcgggccagctcgaaagcatcgcgcggccctttcagcgcttcgtccgcgatcactggcaggctgaaacggcgggtcagtcgcgccagtccggcacgattttctgccgcaatcggctgctcaacggcatcaatgccgccggcttccagtgctgccattccgcgctcggcctggcgctcgctccaggcctggttgacgtcgacgcgcacgctgacctcatcccccaacgcctgcctgatggccagcgcgtgcgccacgtccgcatcaacatcgcgcaggccgattttgagtttaaaaatacgatgacggcgcagggctaacatctgccgggcttcggcaatatctttatcggtgctgccgctggccaacgtccaggccaccggtagcgcatcgcgcacccgtcctcccagcagttcactgagcgcaatattcagacgccgtgcctgagcgtccagcaaggcggtttcaattgcgcatttagcaaagcggttgccctgcactgatttattcaggcgcgccatcagctgggcgatgcggcgcgcatcctgaccgatcagtaacggcgtcatccagcggtcaatattgactttgacgctctcggggctttcgtcgccgtagctcaggccaccaatggtggtggcttctccccagccctcaaacccgtcctcacacaccagatggaccagcaccagcgtttgcacctgaagcgtggccattgccaggcgatgtggacggatagcaggaacatcaataagcagcgtttcaatagttttgatcat

SEQ ID NO: 4 - protein, 369 aa, Erwinia aphidicola
  REGION 1..369: note = misc_feature - LJJL01Muconate cycloisomerase protein sequence (Gene ID:2752379084)
MIKTIETLLIDVPAIRPHRLAMATLQVQTLVLVHLVCEDGFEGWGEATTIGGLSYGDESPESVKVNIDRWMTPLLIGQDARRIAQLMARLNKSVQGNRFAKCAIETALLDAQARRLNIALSELLGGRVRDALPVAWTLASGSTDKDIAEARQMLALRRHRIFKLKIGLRDVDADVAHALAIRQALGDEVSVRVDVNQAWSERQAERGMAALEAGGIDAVEQPIAAENRAGLARLTRRFSLPVIADEALKGPRDAFELARHAAADVFSIKITQSGGLTQARRVADIAQLADIALYGGTMLEGAVGTAATAHLCATFNDLSFGTELFGPLLLTEDILSEPLVYRDFMLQVPTGPGLGIALDRDRIAALRRR

SEQ ID NO: 5 - protein, 320 aa, unidentified
  REGION 1..320: note = Bacteria, LCC protein sequence
MNFPRASRLMQAAVLGGLMAVSAAATAMDGVLWRVRTAALMAALLALAAWALVWASPSVEAQSNPYQRGPNPTRSALTADGPFSVATYTVSRLSVSGFGGGVIYYPTGTSLTFGGIAMSPGYTADASSLAWLGRRLASHGFVVLVINTNSRFDGPDSRASQLSAALNYLRTSSPSAVRARLDANRLAVAGHSMGGGGTLRIAEQNPSLKAAVPLTPWHTDKTFNTSVPVLIVGAEADTVAPVSQHAIPFYQNLPSTTPKVYVELCNASHIAPNSNNAAISVYTISWMKLWVDNDTRYRQFLCNVNDPALCDFRTNNRHCQ

SEQ ID NO: 6 - protein, 613 aa, unidentified
  REGION 1..613: note = Bacteria, MHET protein sequence
MQTTVTTMLLASVALAACAGGGSTPLPLPQQQPPQQEPPPPPVPLASRAACEALKDGNGDMVWPNAATVVEVAAWRDAAPATASAAALPEHCEVSGAIAKRTGIDGYPYEIKFRLRMPAEWNGRFFMEGGSGTNGSLSAATGSIGGGQIASALSRNFATIATDGGHDNAVNDNPDALGTVAFGLDPQARLDMGYNSYDQVTQAGKAAVARFYGRAADKSYFIGCSEGGREGMMLSQRFPSHYDGIVAGAPGYQLPKAGISGAWTTQSLAPAAVGLDAQGVPLINKSFSDADLHLLSQAILGTCDALDGLADGIVDNYRACQAAFDPATAANPANGQALQCVGAKTADCLSPVQVTAIKRAMAGPVNSAGTPLYNRWAWDAGMSGLSGTTYNQGWRSWWLGSFNSSANNAQRVSGFSARSWLVDFATPPEPMPMTQVAARMMKFDFDIDPLKIWATSGQFTQSSMDWHGATSTDLAAFRDRGGKMILYHGMSDAAFSALDTADYYERLGAAMPGAAGFARLFLVPGMNHCSGGPGTDRFDMLTPLVAWVERGEAPDQISAWSGTPGYFGVAARTRPLCPYPQIARYKGSGDINTEANFACAAPPIIQDEPQTPA

SEQ ID NO: 7 - DNA, 5410 bp, synthetic construct
  misc_feature 1..5410: note = Synthetic: pBLT-2:Ptac-LCC_MHETase plasmid gene sequence
  source 1..5410: mol_type = other DNA
atcattcaggacgagcctcagactccagcgtaactggactgaaaacaaactaaagcgcccttgtggcgctttagttttgttccgcggccaccggctggctcgcttcgctcggcccgtggacaaccctgctggacaagctgatggacaggctgcgcctgcccacgagcttgaccacagggattgcccaccggctacccagccttcgaccacatacccaccggctccaactgcgcggcctgcggccttgccccatcaatttttttaattttctctggggaaaagcctccggcctgcggcctgcgcgcttcgcttgccggttggacaccaagtggaaggcgggtcaaggctcgcgcagcgaccgcgcagcggcttggccttgacgcgcctggaacgacccaagcctatgcgagtgggggcagtcgaaggcgaagcccgcccgcctgccccccgagcctcacggcggcgagtgcgggggttccaagggggcagcgccaccttgggcaaggccgaaggccgcgcagtcgatcaacaagccccggaggggccactttttgccggagggggagccgcgccgaaggcgtgggggaaccccgcaggggtgcccttctttgggcaccaaagaactagatatagggcgaaatgcgaaagacttaaaaatcaacaacttaaaaaaggggggtacgcaacagctcattgcggcaccccccgcaatagctcattgcgtaggttaaagaaaatctgtaattgactgccacttttacgcaacgcataattgttgtcgcgctgccgaaaagttgcagctgattgcgcatggtgccgcaaccgtgcggcaccctaccgcatggagataagcatggccacgcagtccagagaaatcggcattcaagccaagaacaagcccggtcactgggtgcaaacggaacgcaaagcgcatgaggcgtgggccgggcttattgcgaggaaacccacggcggcaatgctgctgcatcacctcgtggcgcagatgggccaccagaacgccgtggtggtcagccagaagacactttccaagctcatcggacgttctttgcggacggtccaatacgcagtcaaggacttggtggccgagcgctggatctccgtcgtgaagctcaacggccccggcaccgtgtcggcctacgtggtcaatgaccgcgtggcgtggggccagccccgcgaccagttgcgcctgtcggtgttcagtgccgccgtggtggttgatcacgacgaccaggacgaatcgctgttggggcatggcgacctgcgccgcatcccgaccctgtatccgggcgagcagcaactaccgaccggccccggcgaggagccgcccagccagcccggcattccgggcatggaaccagacctgccagccttgaccgaaacggaggaatgggaacggcgcgggcagcagcgcctgccgatgcccgatgagccgtgttttctggacgatggcgagccgttggagccgccgacacgggtcacgctgccgcgccggtagtacgtaagaggttccaactttcaccataatgaaataagatcactaccgggcgtattttttgagttatcgagattttcaggagctaaggaagctaaaatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcaggctaaactggctgacggaatttatgcctcttccgaccatcaagcattttatccgtactcctgatgatgcatggttactcaccactgcgatcccagggaaaacagcattccaggtattagaagaatatcctgattcaggtgaaaatattgttgatgcgctggcagtgttcctgcgccggttgcattcgattcctgtttgtaattgtccttttaacggcgatcgcgtatttcgtctcgctcaggcgcaatcacgaatgaataacggtttggttggtgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcacttgatgctcgatgagtttttctgagggcggatccccctcaagtcaaaagcctccggtcggaggcttttgactttctgctatggaggtcaggtatgattacactctagagagctgttgacaattaatcatcggctcgtataatgtgtggaattgtgagcggataacaatttcacaccatcaagtcaaaacactatataggaacgaaaccatgaacttccctcgcgcgtcgcgcctgatgcaggcggcggtcctcggtggtctgatggcagtcagcgccgcggccaccgctatggacggtgtactgtggcgtgtgcgcaccgcagccctgatggccgccctgctggcactggctgcgtgggcgctggtgtgggcgtcgccgagcgttgaggcgcagagcaacccttaccagcgtggcccgaacccgacccgctcggccctgaccgccgacggtccattctctgtcgccacctataccgtcagccgcctgtcggtgtcgggtttcggcggcggcgtgatctactacccgaccggcacctccttgaccttcggcggcattgccatgtcccctggctacaccgctgacgcctcgagcctggcctggctgggtcgccgtctggccagccacggcttcgtcgtactggtcatcaacaccaacagccgtttcgacggccctgactcccgcgccagccagctgtcggctgctctgaactacctgcgtacctcgtcgccgtccgcagtccgtgcccgcctggacgccaaccgtctggccgtagccggccactcgatgggcggcggcggcaccctgcgcatcgccgaacaaaacccaagcctgaaggccgctgtcccactgaccccgtggcacaccgacaagaccttcaacacctcggtgccagtgctgatcgtaggtgcggaggccgacaccgtggccccagtgtcccagcacgccatcccgttctaccagaacctgccgtccaccaccccaaaggtttatgtcgagctctgcaacgccagtcacatcgccccgaattcgaacaacgctgccatctccgtctacaccatcagctggatgaagctgtgggttgacaacgacacccgctaccgccagttcctgtgtaacgtgaacgacccggccctgtgcgatttccgtaccaacaatcgtcactgccagtgacaaggattacatataagggtatataaatgcagaccaccgtcaccactatgctgctggcatcggtcgccctggccgcctgcgcaggcggcggcagcaccccgctgccgctgccgcagcaacagccgccacagcaggagccgccgcctcctccagtcccgctggcttcccgtgctgcgtgtgaggccctgaaggacggcaacggggacatggtttggccgaacgccgccaccgtagttgaagtggccgcatggcgcgacgctgccccggctaccgcgtccgccgccgctctgccggaacactgcgaagttagcggcgccatcgccaagcgcactggtattgacggttatccgtacgaaatcaagttccgcctgcgcatgccggcggagtggaatggccgtttcttcatggagggtggttccggcaccaacggctccctgagcgcggccaccggcagcatcggtggcggccagatcgcctcggccctgtcccgcaacttcgccaccatcgcgaccgacggtggccacgacaacgctgtcaacgacaatccagacgccctgggtacggtagcgttcggcctggacccacaggctcgcctggacatgggttacaattcgtacgaccaggtgacccaagctggcaaagccgccgttgcccgtttctacggccgtgccgccgacaagtcgtacttcatcggctgctcggaaggtggtcgggagggcatgatgctcagccaacgcttcccatcccactacgacggtatcgtcgccggtgcccctggctaccagctgcctaaagccggtatctcgggcgcttggaccactcagtcgctggccccggcggcggtgggcctggacgctcagggcgtcccgctgatcaacaagagcttctccgatgccgacctgcacctgctgtcgcaggccatcctcggtacttgcgatgcgctggacggcctggctgacggcatcgttgacaactaccgcgcgtgccaggccgctttcgacccggctaccgcggctaaccctgccaacggtcaagctctgcaatgtgtgggtgccaaaaccgccgattgcctgagcccggtacaggttaccgccatcaaacgtgcaatggccggcccggtcaacagcgccggcaccccgctgtacaaccgttgggcctgggacgctggtatgagcggcctgtccggtaccacctacaatcagggctggcgttcctggtggctgggtagcttcaactcctcggcgaacaacgcgcagcgtgtttcgggtttctccgcccgctcctggctggtcgacttcgccaccccaccagagcctatgccgatgacccaggtggctgcacgcatgatgaaattcgacttcgacatcgacccgctgaagatctgggccaccagcggccagttcacccagtcgagcatggactggcacggggccacctccaccgacctggccgccttccgcgatcgtggcggcaagatgatcctgtaccacggtatgagcgacgcagccttctcggccctggacaccgctgactactacgaacgcctgggcgccgctatgccgggcgccgcgggcttcgctcgtctgttcctcgtcccaggcatgaaccactgttcgggcggtccaggtaccgaccgtttcgacatgctgacccctctggtggcgtgggttgagcgcggcgaagccccggaccagatctcggcgtggagcggcaccccaggctacttcggcgtcgctgcccgtacccgcccgctgtgcccgtacccgcaaatcgcacgctacaagggttccggcgatatcaacaccgaagcaaacttcgcctgcgccgcgcctccg

SEQ ID NO: 8 - DNA, 1070 bp, synthetic construct
  misc_feature 1..1070: note = Synthetic: new synthetic LCC expression cassette
  source 1..1070: mol_type = other DNA
ctctagagagctgttgacaattaatcatcggctcgtataatgtgtggaattgtgagcggataacaatttcacaccatcaagtcaaaacactatataggaacgaaaccatgaacttccctcgcgcgtcgcgcctgatgcaggcggcggtcctcggtggtctgatggcagtcagcgccgcggccaccgctatggacggtgtactgtggcgtgtgcgcaccgcagccctgatggccgccctgctggcactggctgcgtgggcgctggtgtgggcgtcgccgagcgttgaggcgcagagcaacccttaccagcgtggcccgaacccgacccgctcggccctgaccgccgacggtccattctctgtcgccacctataccgtcagccgcctgtcggtgtcgggtttcggcggcggcgtgatctactacccgaccggcacctccttgaccttcggcggcattgccatgtcccctggctacaccgctgacgcctcgagcctggcctggctgggtcgccgtctggccagccacggcttcgtcgtactggtcatcaacaccaacagccgtttcgacggccctgactcccgcgccagccagctgtcggctgctctgaactacctgcgtacctcgtcgccgtccgcagtccgtgcccgcctggacgccaaccgtctggccgtagccggccactcgatgggcggcggcggcaccctgcgcatcgccgaacaaaacccaagcctgaaggccgctgtcccactgaccccgtggcacaccgacaagaccttcaacacctcggtgccagtgctgatcgtaggtgcggaggccgacaccgtggccccagtgtcccagcacgccatcccgttctaccagaacctgccgtccaccaccccaaaggtttatgtcgagctctgcaacgccagtcacatcgccccgaattcgaacaacgctgccatctccgtctacaccatcagctggatgaagctgtgggttgacaacgacacccgctaccgccagttcctgtgtaacgtgaacgacccggccctgtgcgatttccgtaccaacaatcgtcactgccagtga

SEQ ID NO: 9 - DNA, 4965 bp, synthetic construct
  misc_feature 1..4965: note = Synthetic: ACE-540972 plasmid gene sequence
  source 1..4965: mol_type = other DNA
atggccacgcagtccagagaaatcggcattcaagccaagaacaagcccggtcactgggtgcaaacggaacgcaaagcgcatgaggcgtgggccgggcttattgcgaggaaacccacggcggcaatgctgctgcatcacctcgtggcgcagatgggccaccagaacgccgtggtggtcagccagaagacactttccaagctcatcggacgttctttgcggacggtccaatacgcagtcaaggacttggtggccgagcgctggatctccgtcgtgaagctcaacggccccggcaccgtgtcggcctacgtggtcaatgaccgcgtggcgtggggccagccccgcgaccagttgcgcctgtcggtgttcagtgccgccgtggtggttgatcacgacgaccaggacgaatcgctgttggggcatggcgacctgcgccgcatcccgaccctgtatccgggcgagcagcaactaccgaccggccccggcgaggagccgcccagccagcccggcattccgggcatggaaccagacctgccagccttgaccgaaacggaggaatgggaacggcgcgggcagcagcgcctgccgatgcccgatgagccgtgttttctggacgatggcgagccgttggagccgccgacacgggtcacgctgccgcgccggtagtacgtaagaggttccaactttcaccataatgaaataagatcactaccgggcgtattttttgagttatcgagattttcaggagctaaggaagctaaaatgagccatattcaacgggaaacgtcttgctcgaggccgcgattaaattccaacatggatgctgatttatatgggtataaatgggctcgcggcaatcaggtgcgacaatctatcgattgtatgggaagcccgatgcgccagagttgtttctgaaacatggcaaaggtagcgttgccaatgatgttacagatgagatggtcaggctaaactggctgacggaatttatgcctcttccgaccatcaagcattttatccgtactcctgatgatgcatggttactcaccactgcgatcccagggaaaacagcattccaggtattagaagaatatcctgattcaggtgaaaatattgttgatgcgctggcagtgttcctgcgccggttgcattcgattcctgtttgtaattgtccttttaacggcgatcgcgtatttcgtctcgctcaggcgcaatcacgaatgaataacggtttggttggtgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcacttgatgctcgatgagtttttctgagggcggatccccctcaacggatccccctcaagtcaaaagcctccggtcggaggcttttgactttctgctatggaggtcaggtatgattacactctagagagctgttgacaattaatcatcggctcgtataatgtgtggaattgtgagcggataacaatttcacaccatcaagtcaaaacactatataggaacgaaaccatgaacttccctcgcgcgtcgcgcctgatgcaggcggcggtcctcggtggtctgatggcagtcagcgccgcggccaccgctatgtgcctgagcctggcggcactgctgaacgcggttctggtgctgcgcgccgcaccggccctgggtagcccggcaaagcgtagctgctcccaggcagatctgaccgtggagctggacagcggcatcgttcacggcaccgtggagagcggcaacccggatgttcgtcagttcctgggtatcccgtacgcgaaaccgccggtgggtgacctgcgcttcgcaccgagcgagccgattgagagcttcggcgagatcaccgcggataccctgccgccgagctgtatgcagtacctgaccagcctggagagcatctggaccatcgacgtgctgcaattcaacgaagcgggtctgaacaccaccggtccgcagagcgaggattgcctgaccatcagcgtgtgggcgccgcgtggcgccaagaaagacctgccggtcctgctgtggatctacggcggtagcttcaagaccggcggtgaggatgtcccgtaccagatcccgacccagtgggttcagcgtacccaggaccacatcgtggtcagcttcaactaccgcgtcaacatcttcggtttcccgaacgcggccggtctggacgatgacaagcagaacctgggcctgctggatcagcgtctggcggtcgagtgggttcgcgataacatcgccaaattcggcggtgacgttaaccgtatcggtctgtggggtcagagcgcgggcggtatcagcgtcgcctactacagctacacctacccggaggacccgattgtgagcgcgctgctgatgaacagcggtaacgagtacctggatatcaccagcagcgacaccacccacagcaacttcaccttcatggccagccagttcggttgcggtgatctggccccggatgaggagctggcgtgtatgcgtaccgttgatgcgagcgccatcgaggagttcctgcacatctacatcgacaacggcaccgagccggcggtgagcttcgcaccgattgtggacgagaagaccgtcttcagcgatttctacgaccgcgccgtcaacggtagcgttgccgcactgccgaccatcctgggcagcaacctggatgacggtgtgccgttcgtcacctacagcccggatggcgttaacgcgaccgacgcctacgaggtgaccaccgactacttcttctgcccggcgttcaagagcgccaacaaccgtcacgcggccggtgcgccggtgttccgctacgagtacagcggcaacttcagcaacatcagcccgaaaccgtggatgggtgcctggcacaacagcgagctgccgctgctgttcggcacctacagcaactaccgtggtccgagcaccagcctggagatcgagaccagcatcgcgatgcaggatgcctggctgagcctggtcgcgaacggtgcggccggtccgctggccctgggttggccgctgtacagcccggaggcgggcagcctgctgcgcgatttcggtaaagacgtggcggcccagaccaccaacttcgaggactgggagagcacctgctccgaggtgttccagccgggaggtggaggaggttctatgagcaagggcgaggagctctttaccggcgtcgtccccattctcgttgagctggacggcgacgtgaacggacataagttcagtgtctcgggcgagggcgaaggagatgccacctatgggaagctaaccctgaagttcatctgcacaaccgggaagctgccggtcccctggccgacgctggttaccaccctgacctacggcgtgcaatgcttctcgcgctaccctgaccacatgaagcgccacgacttcttcaaatccgctatgccggagggctacgtccaggaacgcaccatattcttcaaggacgacggtaactacaagacgcgcgccgaagtcaagttcgagggggataccctcgtgaaccgaatcgagttgaaggggatcgacttcaaagaagatggcaacatcctcggccacaaactggagtacaactacaattcgcataacgtgtacatcatggccgacaagcagaagaatggcatcaaggtgaacttcaagattcgccacaacatcgaggacgggtccgttcagctggccgaccactatcagcagaacacaccaattggagacggccccgtcctgctccccgataaccattacctttcgacacagtcggcgctgtcgaaggacccgaacgaaaagcgggaccacatggtgctcctggagttcgtcacggcggccgggatcacgcacggaatggacgaactctacaagtaggatatcattcaggacgagcctcagactccagcgtaactggactgaaaacaaactaaagcgcccttgtggcgctttagttttgttccgcggccaccggctggctcgcttcgctcggcccgtggacaaccctgctggacaagctgatggacaggctgcgcctgcccacgagcttgaccacagggattgcccaccggctacccagccttcgaccacatacccaccggctccaactgcgcggcctgcggccttgccccatcaatttttttaattttctctggggaaaagcctccggcctgcggcctgcgcgcttcgcttgccggttggacaccaagtggaaggcgggtcaaggctcgcgcagcgaccgcgcagcggcttggccttgacgcgcctggaacgacccaagcctatgcgagtgggggcagtcgaaggcgaagcccgcccgcctgccccccgagcctcacggcggcgagtgcgggggttccaagggggcagcgccaccttgggcaaggccgaaggccgcgcagtcgatcaacaagccccggaggggccactttttgccggagggggagccgcgccgaaggcgtgggggaaccccgcaggggtgcccttctttgggcaccaaagaactagatatagggcgaaatgcgaaagacttaaaaatcaacaacttaaaaaaggggggtacgcaacagctcattgcggcaccccccgcaatagctcattgcgtaggttaaagaaaatctgtaattgactgccacttttacgcaacgcataattgttgtcgcgctgccgaaaagttgcagctgattgcgcatggtgccgcaaccgtgcggcaccctaccgcatggagataagc

SEQ ID NO: 10 - DNA, 4978 bp, synthetic construct
  misc_feature 1..4978: note = Synthetic: LAP_117292 plasmid gene sequence
  source 1..4978: mol_type = other DNA
atggccacgcagtccagagaaatcggcattcaagccaagaacaagcccggtcactgggtgcaaacggaacgcaaagcgcatgaggcgtgggccgggcttattgcgaggaaacccacggcggcaatgctgctgcatcacctcgtggcgcagatgggccaccagaacgccgtggtggtcagccagaagacactttccaagctcatcggacgttctttgcggacggtccaatacgcagtcaaggacttggtggccgagcgctggatctccgtcgtgaagctcaacggccccggcaccgtgtcggcctacgtggtcaatgaccgcgtggcgtggggccagccccgcgaccagttgcgcctgtcggtgttcagtgccgccgtggtggttgatcacgacgaccaggacgaatcgctgttggggcatggcgacctgcgccgcatcccgaccctgtatccgggcgagcagcaactaccgaccggccccggcgaggagccgcccagccagcccggcattccgggcatggaaccagacctgccagccttgaccgaaacggaggaatgggaacggcgcgggcagcagcgcctgccgatgcccgatgagccgtgttttctggacgatggcgagccgttggagccgccgacacgggtcacgctgccgcgccggtagtacgtaagaggttccaactttcaccataatgaaataagatcactaccgggcgtattttttgagttatcgagattttcaggagctaaggaagctaaaatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcaggctaaactggctgacggaatttatgcctcttccgaccatcaagcattttatccgtactcctgatgatgcatggttactcaccactgcgatcccagggaaaacagcattccaggtattagaagaatatcctgattcaggtgaaaatattgttgatgcgctggcagtgttcctgcgccggttgcattcgattcctgtttgtaattgtccttttaacggcgatcgcgtatttcgtctcgctcaggcgcaatcacgaatgaataacggtttggttggtgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcacttgatgctcgatgagtttttctgagggcggatccccctcaacggatccccctcaagtcaaaagcctccggtcggaggcttttgactttctgctatggaggtcaggtatgattacactctagagagctgttgacaattaatcatcggctcgtataatgtgtggaattgtgagcggataacaatttcacaccatcaagtcaaaacactatataggaacgaaaccatgaacttccctcgcgcgtcgcgcctgatgcaggcggcggtcctcggtggtctgatggcagtcagcgccgcggccaccgctatgcgtgtcaccctgggtctggcggccctggccgttggtgccagcgccatcccgaccgagctgcgtgagcgtcaggacggccacaacagcaaaccgtgcccgaagaaaccgctggtgaccaaagacgccctggagaaggatatcaccatcaagaaactgatgcgcggtgcgcagcagctggaggatttcgcctacagctacccggcgcgtaaccgcatcatgggcggtcaggcgcacaacgacaccgtcaagtggctgaagaaagagctggagagcaccagctactacgatgtgaccctgcaaccgttcagcaactacgtcatgctgaacggcaccctgaacgccttcaccatcgacggtcgtgcgatcaacagcaccatcctggagtacagcgccagcaccccgggtctggttaccgccccgattgtgccggtgaacaacctgggttgcgaggccagcgatttcccggccgaggtgtccggtgccatcgcgctgatcagccgtggcacctgcgagttcggtctgaaaagcgccctggcgggtagcgccggcgccgttggtgccatcatctacaacaacgtggccggcaccatcagcgccaccctgggcccgccgccgcgtccggagggtgactacgtggcgagcgtcaccctgagcctggaggaaggccaggccatcgttgccaaagtggccggcggtgcgaccgtgaccggcaccctggacgtcctgaccgatgtccaggttatcaccaccaacaacgtcctggcgaccagcaaatgcggcgacaagaacaaccagctgtggctgggtgcccacaccgatagcgttggtgccggtccgggtatcaacgacgatggtagcggcaccgttgccatcctgaacgtggccaaaagcctggcgaagtacaacgtgaacaacgcggtcagcttcggtttctggagcggtgaagagagcggtctgctgggtagcaccttcttcgtggagagcctgagcccggaggccgccctggacgttcgtgcctacctgaacttcgatatgatcgcgagcccgaactacgtgcaccagatctacgatggtgatggcagcgcctacggcctgagcggtccggcgggcagcgatgatatcgaggccttcttcgcgggttacttcaaagacctgaacatcccgagcaacgagaccgagttcaacggtcgcagcgactacggcccgttcctggatgcaaacatcccggcgggcggcaccaccaccggtgcagatgaggtcaagaccgttgaagagcaggccatctggggcggtgtggccggcgagatcctggaccagaactaccaccaggcggccgataacgtcaccaacctgaacgaggaagcgtggctgctgcacagccgcggcatcgcggcggcggtggcccactacgccaccagctgggaaggcatcccggagcgtgttccggcaaacggcaccgccaaacgtagcgtgcgtgagccggtggtccgtaagggtgtcaaaggcggtaaagttaccctgaagctgctgggaggtggaggaggttctatgagcaagggcgaggagctctttaccggcgtcgtccccattctcgttgagctggacggcgacgtgaacggacataagttcagtgtctcgggcgagggcgaaggagatgccacctatgggaagctaaccctgaagttcatctgcacaaccgggaagctgccggtcccctggccgacgctggttaccaccctgacctacggcgtgcaatgcttctcgcgctaccctgaccacatgaagcgccacgacttcttcaaatccgctatgccggagggctacgtccaggaacgcaccatattcttcaaggacgacggtaactacaagacgcgcgccgaagtcaagttcgagggggataccctcgtgaaccgaatcgagttgaaggggatcgacttcaaagaagatggcaacatcctcggccacaaactggagtacaactacaattcgcataacgtgtacatcatggccgacaagcagaagaatggcatcaaggtgaacttcaagattcgccacaacatcgaggacgggtccgttcagctggccgaccactatcagcagaacacaccaattggagacggccccgtcctgctccccgataaccattacctttcgacacagtcggcgctgtcgaaggacccgaacgaaaagcgggaccacatggtgctcctggagttcgtcacggcggccgggatcacgcacggaatggacgaactctacaagtaggatatcattcaggacgagcctcagactccagcgtaactggactgaaaacaaactaaagcgcccttgtggcgctttagttttgttccgcggccaccggctggctcgcttcgctcggcccgtggacaaccctgctggacaagctgatggacaggctgcgcctgcccacgagcttgaccacagggattgcccaccggctacccagccttcgaccacatacccaccggctccaactgcgcggcctgcggccttgccccatcaatttttttaattttctctggggaaaagcctccggcctgcggcctgcgcgcttcgcttgccggttggacaccaagtggaaggcgggtcaaggctcgcgcagcgaccgcgcagcggcttggccttgacgcgcctggaacgacccaagcctatgcgagtgggggcagtcgaaggcgaagcccgcccgcctgccccccgagcctcacggcggcgagtgcgggggttccaagggggcagcgccaccttgggcaaggccgaaggccgcgcagtcgatcaacaagccccggaggggccactttttgccggagggggagccgcgccgaaggcgtgggggaaccccgcaggggtgcccttctttgggcaccaaagaactagatatagggcgaaatgcgaaagacttaaaaatcaacaacttaaaaaaggggggtacgcaacagctcattgcggcaccccccgcaatagctcattgcgtaggttaaagaaaatctgtaattgactgccacttttacgcaacgcataattgttgtcgcgctgccgaaaagttgcagctgattgcgcatggtgccgcaaccgtgcggcaccctaccgcatggagataagc

SEQ ID NO: 11 - DNA, 5335 bp, synthetic construct
  misc_feature 1..5335: note = Synthetic: pBLT-2-LCC-Mle046 plasmid gene sequence
  source 1..5335: mol_type = other DNA
gtgtggccaataagctttgactggactgaaaacaaactaaagcgcccttgtggcgctttagttttgttccgcggccaccggctggctcgcttcgctcggcccgtggacaaccctgctggacaagctgatggacaggctgcgcctgcccacgagcttgaccacagggattgcccaccggctacccagccttcgaccacatacccaccggctccaactgcgcggcctgcggccttgccccatcaatttttttaattttctctggggaaaagcctccggcctgcggcctgcgcgcttcgcttgccggttggacaccaagtggaaggcgggtcaaggctcgcgcagcgaccgcgcagcggcttggccttgacgcgcctggaacgacccaagcctatgcgagtgggggcagtcgaaggcgaagcccgcccgcctgccccccgagcctcacggcggcgagtgcgggggttccaagggggcagcgccaccttgggcaaggccgaaggccgcgcagtcgatcaacaagccccggaggggccactttttgccggagggggagccgcgccgaaggcgtgggggaaccccgcaggggtgcccttctttgggcaccaaagaactagatatagggcgaaatgcgaaagacttaaaaatcaacaacttaaaaaaggggggtacgcaacagctcattgcggcaccccccgcaatagctcattgcgtaggttaaagaaaatctgtaattgactgccacttttacgcaacgcataattgttgtcgcgctgccgaaaagttgcagctgattgcgcatggtgccgcaaccgtgcggcaccctaccgcatggagataagcatggccacgcagtccagagaaatcggcattcaagccaagaacaagcccggtcactgggtgcaaacggaacgcaaagcgcatgaggcgtgggccgggcttattgcgaggaaacccacggcggcaatgctgctgcatcacctcgtggcgcagatgggccaccagaacgccgtggtggtcagccagaagacactttccaagctcatcggacgttctttgcggacggtccaatacgcagtcaaggacttggtggccgagcgctggatctccgtcgtgaagctcaacggccccggcaccgtgtcggcctacgtggtcaatgaccgcgtggcgtggggccagccccgcgaccagttgcgcctgtcggtgttcagtgccgccgtggtggttgatcacgacgaccaggacgaatcgctgttggggcatggcgacctgcgccgcatcccgaccctgtatccgggcgagcagcaactaccgaccggccccggcgaggagccgcccagccagcccggcattccgggcatggaaccagacctgccagccttgaccgaaacggaggaatgggaacggcgcgggcagcagcgcctgccgatgcccgatgagccgtgttttctggacgatggcgagccgttggagccgccgacacgggtcacgctgccgcgccggtagtacgtaagaggttccaactttcaccataatgaaataagatcactaccgggcgtattttttgagttatcgagattttcaggagctaaggaagctaaaatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcaggctaaactggctgacggaatttatgcctcttccgaccatcaagcattttatccgtactcctgatgatgcatggttactcaccactgcgatcccagggaaaacagcattccaggtattagaagaatatcctgattcaggtgaaaatattgttgatgcgctggcagtgttcctgcgccggttgcattcgattcctgtttgtaattgtccttttaacggcgatcgcgtatttcgtctcgctcaggcgcaatcacgaatgaataacggtttggttggtgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcacttgatgctcgatgagtttttctgagggcggatccccctcaagtcaaaagcctccggtcggaggcttttgactttctgctatggaggtcaggtatgattacactctagagagctgttgacaattaatcatcggctcgtataatgtgtggaattgtgagcggataacaatttcacaccatcaagtcaaaacactatataggaacgaaaccatgaacttccctcgcgcgtcgcgcctgatgcaggcggcggtcctcggtggtctgatggcagtcagcgccgcggccaccgctatggacggtgtactgtggcgtgtgcgcaccgcagccctgatggccgccctgctggcactggctgcgtgggcgctggtgtgggcgtcgccgagcgttgaggcgcagagcaacccttaccagcgtggcccgaacccgacccgctcggccctgaccgccgacggtccattctctgtcgccacctataccgtcagccgcctgtcggtgtcgggtttcggcggcggcgtgatctactacccgaccggcacctccttgaccttcggcggcattgccatgtcccctggctacaccgctgacgcctcgagcctggcctggctgggtcgccgtctggccagccacggcttcgtcgtactggtcatcaacaccaacagccgtttcgacggccctgactcccgcgccagccagctgtcggctgctctgaactacctgcgtacctcgtcgccgtccgcagtccgtgcccgcctggacgccaaccgtctggccgtagccggccactcgatgggcggcggcggcaccctgcgcatcgccgaacaaaacccaagcctgaaggccgctgtcccactgaccccgtggcacaccgacaagaccttcaacacctcggtgccagtgctgatcgtaggtgcggaggccgacaccgtggccccagtgtcccagcacgccatcccgttctaccagaacctgccgtccaccaccccaaaggtttatgtcgagctctgcaacgccagtcacatcgccccgaattcgaacaacgctgccatctccgtctacaccatcagctggatgaagctgtgggttgacaacgacacccgctaccgccagttcctgtgtaacgtgaacgacccggccctgtgcgatttccgtaccaacaatcgtcactgccagtgacaaggattacatataagggtatataaatgaacttccctcgcgcgtcgcgcctgatgcaggcggcggtcctcggtggtctgatggcagtcagcgccgcggccaccgctcatatggcagttgcaggtgacttcccgaatcagttcctgagctgcttcgatgcagccaatctgaccgaaattgaactgccggcagatgttcagggcttccgtctgattgaaattgccgaacatgccggtgataaaggcatgccggcccattgtgaaattgtgggcgccattaatgatcgcattagtccggtggatggccagcattatagcattaaattccgtctgcgtctgccgcaggattggaatggtcgcttctatatggaaggcggtggcggtagcaatggtgttctgaaagatgcaatgggcccgaccggcctgaatcaggaagatagtgccctggaacgcggcttcgcagtggtgaccaccgatagcggccatgataatgataccaatagcgatagcaatgccagcggccgcagcgccttcggtatggatccgcaggcacgcctggacttcggttatatgagttatgatattgttacccgtgtgggtaaagcaattgtggaaaaatattatggtgcagccccggaaaaaagttacttcattggttgtagtgaaggcggtcgtgaagcagccctgatgacccagcgctatccggatctgtatgatggtgtggttgccggtgccccgggtattcacttcagctatagtgcagcctatgccccgttcctgctgcgcatcttcggtaatctggccgaaaccagaaatcagagcggtccggatggtattccgctgctgaataaactgtatagtgataatgatgtgcagctgattgccgatgccgttgtgggcgcctgtgatgccctggatggcctggaagatcgtatgagcaataatattgaagcctgtaccaccgttaccgtgctgccgcgcctgcgcgctctgacatgcagtggcgccaaagaagaaggttgcctgctggaagatcagattgatgccttcgtggccggtatggcaggtccggtgaccagtgatggtacccgtctgtatccgggtcatccgtgggatccgggtattggcggtcgtattggtgatagtgttaatgatggcttccgtagttggtggttcggtagctatgatagcgatcagaataatgcccgcaaagtgaccctgagcaccccgcagcatgccatgctgtggcagaccccgccggttccgctgcgtcctgatgaatatgtgcgcttcgaaatgaacttcaatattgatgaaacacctgccctggcatacgctaccaccgatctgtatccggtgagcagtgcagaactgggcaatgccgatagtccggatctgagcgacttcgcaagccgcggcggtaaactggtgatctatcatggtgccgcagatgcagcattcagtgcactggataccattaaatattggaatgccgttaatgaaaccgccgatggtcaggccgcagacttcgcacgtctgttcattattccgggtatgaatcattgccagggtggtccggccaccgatgatgttgatctgctgaccccgctgatggcatgggttgaagatgatctgccgattgaacgcctggaagccaccgtgagcaatccggattacttcggtggtaaaaatctgagtcgcccgctgtgtccgtatccgctgtatgccgaatatgatggtgaaggtgatccgagtagcgcagaatcattcacct

SEQ ID NO: 12 - DNA, 1957 bp, synthetic construct
  misc_feature 1..1957: note = Synthetic: Mutant Mle046 DNA sequence
  source 1..1957: mol_type = other DNA
ggatccccctcaagtcaaaagcctccggtcggaggcttttgactttctgctatggaggtcaggtatgattttgcattaggcaccccaggcttgacaattaatcatcggctcgtataatgtgtggaattgtgagcggataacaatttcacactctagaggaggaatcttactctagaatgcatatggcagttgcaggtgacttcccgaatcagttcctgagctgcttcgatgcagccaatctgaccgaaattgaactgccggcagatgttcagggcttccgtctgattgaaattgccgaacatgccggtgataaaggcatgccggcccattgtgaaattgtgggcgccattaatgatcgcattagtccggtggatggccagcattatagcattaaattccgtctgcgtctgccgcaggattggaatggtcgcttctatatggaaggcggtggcggtagcaatggtgttctgaaagatgcaatgggcccgaccggcctgaatcaggaagatagtgccctggaacgcagtttcgcagtggtgaccaccgatagcggccatgataatgataccaatagcgatagcaatgccagcggccgcagcgccttcggtatggatccgcaggcacgcctggacttcggttatatgagttatgatattgttacccgtgtgggtaaagcaattgtggaaaaatattatggtgcagccccggaaaaaagttacttcattggttgtagtgaaggcggtcgtgaagcagccctgatgacccagcgctatccggatctgtatgatggtgtggttgccggtgccccgggtattcacttcagctatagtgcagcctatgccccgttcctgctgcgcatcttcggtaatctggccgaaaccagaaatcagagcggtccggatggtattccgctgctgaataaactgtatagtgataatgatgtgcagctgattgccgatgccgttgtgggcgcctgtgatgccctggatggcctggaagatcgtatgagcaataatattgaagcctgtaccaccgttaccgtgctgccgcgcctgcgcgctctgacatgcagtggcgccaaagaagaaggttgcctgctggaagatcagattgatgccttcgtggccggtatggcaggtccggtgaccagtgatggtacccgtctgtatccgggtcatccgtgggatccgggtattggcggtcgtattggtgatagtgttaatgatggcttccgtagttggtggttcggtagctatgatagcgatcagaataatgcccgcaaagtgaccctgagcaccccgcagcatgccatgctgtggcagaccccgccggttccgctgcgtcctgatgaatatgtgcgcttcgaaatgaacttcaatattgatgaaacacctgccctggcatacgctaccaccgatctgtatccggtgagcagtgcagaactgggcaatgccgatagtccggatctgagcgacttcgcaagccgcggcggtaaactggtgatctatcatggtgccgcagatgcagcattcagtgcactggataccattaaatattggaatgccgttaatgaaaccgccgatggtcaggccgcagacttcgcacgtctgttcattattccgggtatgaatcattgccagggtggtccggccaccgatgatgttgatctgctgaccccgctgatggcatgggttgaagatgatctgccgattgaacgcctggaagccaccgtgagcaatccggattacttcggtggtaaaaatctgagtcgcccgctgtgtccgtatccgctgtatgccgaatatgatggtgaaggtgatccgagtagcgcagaatcattcacctgtgtggccaataagctttgagatatcgatatcattcaggacgagcctcagactccagcgtaactggactgaaaacaaactaaagcgcccttgtggcgctttagttttgttcc

SEQ ID NO: 13 - protein, 562 aa, synthetic construct
  REGION 1..562: note = Synthetic: Mle046 protein sequence
MHMAVAGDFPNQFLSCFDAANLTEIELPADVQGFRLIEIAEHAGDKGMPAHCEIVGAINDRISPVDGQHYSIKFRLRLPQDWNGRFYMEGGGGSNGVLKDAMGPTGLNQEDSALERGFAVVTTDSGHDNDTNSDSNASGRSAFGMDPQARLDFGYMSYDIVTRVGKAIVEKYYGAAPEKSYFIGCSEGGREAALMTQRYPDLYDGVVAGAPGIHFSYSAAYAPFLLRIFGNLAETRNQSGPDGIPLLNKLYSDNDVQLIADAVVGACDALDGLEDRMSNNIEACTTVTVLPRLRALTCSGAKEEGCLLEDQIDAFVAGMAGPVTSDGTRLYPGHPWDPGIGGRIGDSVNDGFRSWWFGSYDSDQNNARKVTLSTPQHAMLWQTPPVPLRPDEYVRFEMNFNIDETPALAYATTDLYPVSSAELGNADSPDLSDFASRGGKLVIYHGAADAAFSALDTIKYWNAVNETADGQAADFARLFIIPGMNHCQGGPATDDVDLLTPLMAWVEDDLPIERLEATVSNPDYFGGKNLSRPLCPYPLYAEYDGEGDPSSAESFTCVANKL

SEQ ID NO: 14 - protein, 562 aa, synthetic construct
  REGION 1..562: note = Synthetic: mutant Mle046 (G117S) protein sequence
MHMAVAGDFPNQFLSCFDAANLTEIELPADVQGFRLIEIAEHAGDKGMPAHCEIVGAINDRISPVDGQHYSIKFRLRLPQDWNGRFYMEGGGGSNGVLKDAMGPTGLNQEDSALERSFAVVTTDSGHDNDTNSDSNASGRSAFGMDPQARLDFGYMSYDIVTRVGKAIVEKYYGAAPEKSYFIGCSEGGREAALMTQRYPDLYDGVVAGAPGIHFSYSAAYAPFLLRIFGNLAETRNQSGPDGIPLLNKLYSDNDVQLIADAVVGACDALDGLEDRMSNNIEACTTVTVLPRLRALTCSGAKEEGCLLEDQIDAFVAGMAGPVTSDGTRLYPGHPWDPGIGGRIGDSVNDGFRSWWFGSYDSDQNNARKVTLSTPQHAMLWQTPPVPLRPDEYVRFEMNFNIDETPALAYATTDLYPVSSAELGNADSPDLSDFASRGGKLVIYHGAADAAFSALDTIKYWNAVNETADGQAADFARLFIIPGMNHCQGGPATDDVDLLTPLMAWVEDDLPIERLEATVSNPDYFGGKNLSRPLCPYPLYAEYDGEGDPSSAESFTCVANKL

SEQ ID NO: 15 - DNA, 75 bp, synthetic construct
  misc_feature 1..75: note = Synthetic: oLJ189
  source 1..75: mol_type = other DNA
ggaattgtgagcggataacaatttcacacttcatcaagtcaaaacactatataggaacgaaaccatgaacttccc

SEQ ID NO: 16 - DNA, 48 bp, synthetic construct
  misc_feature 1..48: note = Synthetic: oLJ190
  source 1..48: mol_type = other DNA
cgctggagtctgaggctcgtcctgaatgatcggaggcgcggcgcaggc

SEQ ID NO: 17 - DNA, 21 bp, synthetic construct
  misc_feature 1..21: note = Synthetic: oLJLJ102
  source 1..21: mol_type = other DNA
taacaaggattacatataagg

SEQ ID NO: 18 - DNA, 20 bp, synthetic construct
  misc_feature 1..20: note = Synthetic: oLJLJ103
  source 1..20: mol_type = other DNA
aagtgtgaaattgttatccg

SEQ ID NO: 19 - DNA, 19 bp, synthetic construct
  misc_feature 1..19: note = Synthetic: oLJLJ104
  source 1..19: mol_type = other DNA
atcattcaggacgagcctc

SEQ ID NO: 20 - DNA, 18 bp, synthetic construct
  misc_feature 1..18: note = Synthetic: oLJLJ105
  source 1..18: mol_type = other DNA
ttagctgcagttcgcagt

SEQ ID NO: 21 - DNA, 37 bp, synthetic construct
  misc_feature 1..37: note = Synthetic: oLJLJ120
  source 1..37: mol_type = other DNA
caaggattacatataagggtatataaatgcagaccac

SEQ ID NO: 22 - DNA, 28 bp, synthetic construct
  misc_feature 1..28: note = Synthetic: oLJLJ121
  source 1..28: mol_type = other DNA
aatcatacctgacctccatagcagaaag

SEQ ID NO: 23 - DNA, 46 bp, synthetic construct
  misc_feature 1..46: note = Synthetic: oLJLJ122
  source 1..46: mol_type = other DNA
aggtcaggtatgattacactctagagagctgttgacaattaatcat

SEQ ID NO: 24 - DNA, 38 bp, synthetic construct
  misc_feature 1..38: note = Synthetic: oLJLJ123
  source 1..38: mol_type = other DNA
tatatgtaatccttgtcactggcagtgacgattgttgg

SEQ ID NO: 25 - DNA, 18 bp, synthetic construct
  misc_feature 1..18: note = Synthetic: oLJLJ131
  source 1..18: mol_type = other DNA
atgagcaagggcgaggag

SEQ ID NO: 26 - DNA, 18 bp, synthetic construct
  misc_feature 1..18: note = Synthetic: oLJLJ132
  source 1..18: mol_type = other DNA
ttgagggggatccgccct

SEQ ID NO: 27 - DNA, 42 bp, synthetic construct
  misc_feature 1..42: note = Synthetic: oLJLJ133
  source 1..42: mol_type = other DNA
tgagggcggatccccctcaacggatccccctcaagtcaaaag

SEQ ID NO: 28 - DNA, 57 bp, synthetic construct
  misc_feature 1..57: note = Synthetic: oLJLJ134
  source 1..57: mol_type = other DNA
agctcctcgcccttgctcatagaacctcctccacctccctggcagtgacgattgttg

SEQ ID NO: 29 - DNA, 43 bp, synthetic construct
  misc_feature 1..43: note = Synthetic: oLJLJ183
  source 1..43: mol_type = other DNA
acgagcctcagactccagcgggaggtggaggaggttctatgag

SEQ ID NO: 30 - DNA, 35 bp, synthetic construct
  misc_feature 1..35: note = Synthetic: oLJLJ184
  source 1..35: mol_type = other DNA
gtgctgccgccgcctgcgcaagcggtggccgcggc

SEQ ID NO: 31 - DNA, 35 bp, synthetic construct
  misc_feature 1..35: note = Synthetic: oLJLJ185
  source 1..35: mol_type = other DNA
tcagcgccgcggccaccgcttgcgcaggcggcggc

SEQ ID NO: 32 - DNA, 39 bp, synthetic construct
  misc_feature 1..39: note = Synthetic: oLJLJ186
  source 1..39: mol_type = other DNA
atagaacctcctccacctcccgctggagtctgaggctcg

SEQ ID NO: 33 - DNA, 28 bp, synthetic construct
  misc_feature 1..28: note = Synthetic: oLJLJ187
  source 1..28: mol_type = other DNA
taggatatcattcaggacgagcctcaga

SEQ ID NO: 34 - DNA, 19 bp, synthetic construct
  misc_feature 1..19: note = Synthetic: oLJLJ188
  source 1..19: mol_type = other DNA
agaacctcctccacctccc

SEQ ID NO: 35 - DNA, 34 bp, synthetic construct
  misc_feature 1..34: note = Synthetic: oLJLJ189
  source 1..34: mol_type = other DNA
ggtggaggaggttctatggacggtgtactgtggc

SEQ ID NO: 36 - DNA, 36 bp, synthetic construct
  misc_feature 1..36: note = Synthetic: oLJLJ190
  source 1..36: mol_type = other DNA
ctgaatgatatcctactggcagtgacgattgttggt

SEQ ID NO: 37 - DNA, 22 bp, synthetic construct
  misc_feature 1..22: note = Synthetic: oLJLJ250
  source 1..22: mol_type = other DNA
ggaggtggaggaggttctatgg

SEQ ID NO: 38 - DNA, 32 bp, synthetic construct
  misc_feature 1..32: note = Synthetic: oLJLJ251
  source 1..32: mol_type = other DNA
ggtttcgttcctatatagtgttttgacttgat

SEQ ID NO: 39 - DNA, 33 bp, synthetic construct
  misc_feature 1..33: note = Synthetic: oLJLJ252
  source 1..33: mol_type = other DNA
tataggaacgaaaccatgcagaccaccgtcacc

SEQ ID NO: 40 - DNA, 30 bp, synthetic construct
  misc_feature 1..30: note = Synthetic: oLJLJ253
  source 1..30: mol_type = other DNA
acctcctccacctcccggaggcgcggcgca

SEQ ID NO: 41 - DNA, 39 bp, synthetic construct
  misc_feature 1..39: note = Synthetic: oLJLJ243
  source 1..39: mol_type = other DNA
aggaggtggaggtggaggaggttctatggacggtgtact

SEQ ID NO: 42 - DNA, 23 bp, synthetic construct
  misc_feature 1..23: note = Synthetic: LJ 565
  source 1..23: mol_type = other DNA
cctggaacgcagtttcgcagtgg

SEQ ID NO: 43 - DNA, 21 bp, synthetic construct
  misc_feature 1..21: note = Synthetic: LJ 566
  source 1..21: mol_type = other DNA
gcactatcttcctgattcagg

SEQ ID NO: 44 - protein, 20 aa, Ideonella sakaiansis
  REGION 1..20: note = misc_feature - Signal Peptide One
MNFPRASRLMQAAVLGGLMA

SEQ ID NO: 45 - protein, 24 aa, synthetic construct
  REGION 1..24: note = Synthetic: Signal Peptide Two
VSAAATAMQTTVTTMLLASVALAA

SEQ ID NO: 46 - DNA, 20 bp, synthetic construct
  misc_feature 1..20: note = Synthetic: Spacer sequence
  source 1..20: mol_type = other DNA
tctggcgcagttgatatgta

SEQ ID NO: 47 - DNA, 47 bp, synthetic construct
  misc_feature 1..47: note = Synthetic: Oligonucleotide primer
  source 1..47: mol_type = other DNA
acgaattcgagctcggtacccggggatcctatatgtgccggacgccg

SEQ ID NO: 48 - DNA, 49 bp, synthetic construct
  misc_feature 1..49: note = Synthetic: Oligonucleotide primer
  source 1..49: mol_type = other DNA
cggccagtgccaagcttgcatgcctgcaggttggcaggccgatgccaag

SEQ ID NO: 49 - DNA, 26 bp, synthetic construct
  misc_feature 1..26: note = Synthetic: oLJLJ243A
  source 1..26: mol_type = other DNA
ccacctcccggaggcgcggcgcaggc

(19) United States
(12) Patent Application Publication
(10) Pub. No.: US 2024/0376450 A1
(43) Pub. Date: Nov. 14, 2024

(54) ENGINEERED MICROBES FOR THE PRODUCTION OF BIOPOLYMERS FROM OHD-DERIVED ORGANIC CARBON

(71) Applicant: BOARD OF TRUSTEES OF SOUTHERN ILLINOIS UNIVERSITY, Carbondale, IL (US)

(72) Inventors: Lahiru N. Jayakody, Carbondale, IL (US); Kenneth B. Anderson, Carbondale, IL (US)

(21) Appl. No.: 18/692,182

(22) PCT Filed: Sep. 15, 2022
(86) PCT No.: PCT/US2022/043646

§ 371 Date: Mar. 14, 2024

Related U.S. Application Data
(60) Provisional application No. 63/245,242, filed on Sep. 17, 2021.

(51) Int. Cl. C12N 9/18; B09B 3/60; B09B 101/75; C12P 7/44

(52) U.S. Cl. CPC C12N 9/18; B09B 3/60; C12P 7/44; B09B 2101/75

(57) ABSTRACT

This disclosure generally relates to engineered recombinant bacterial cells producing enzymatic compositions for up-cycling plastics, biomass waste, or co-mixtures of plastics and biomass waste. The disclosure also relates to dual enzyme compositions produced by the recombinant bacterial cells for upcycling plastic materials and/or biomass waste, and methods for upcycling plastics, OHD-treated substrates and/or biomass waste.

### CROSS REFERENCES TO RELATED APPLICATIONS

This application claims the benefit of priority to U.S. Provisional Patent Application 63/245,242 filed Sep. 17, 2021, the entire disclosure of which is herein incorporated by reference in its entirety.

### INCORPORATION-BY-REFERENCE OF SEQUENCE LISTING FILE

A sequence listing submitted herein as an xml file, entitled 1B74388.xml created on Sep. 14, 2022, is incorporated herein in its entirety.

### TECHNICAL FIELD

This disclosure generally relates to engineered recombinant bacterial cells producing enzymatic compositions for upcycling plastics, biomass waste, or co-mixtures of plastics and biomass waste. The disclosure also relates to dual enzyme compositions produced by the recombinant bacterial cells for upcycling plastic materials and/or biomass waste, and methods for upcycling plastics, OHD-treated substrates and/or biomass waste.

### BACKGROUND

Plastics play a critical role in multiple sectors of global economies due to their versatility, advantageous material properties, and low production cost, with the plastics industry accounting for over $400 billion annually in revenue in the United States alone. Many plastics are designed for single-use applications, and as such, plastics account for >15% of the solid waste in landfills in the United States along with a major environmental burden on land and especially in the ocean. Indeed, it is projected that by 2050, there will be more plastics in the ocean than fish on a mass basis. Polyethylene terephthalate (PET) is the largest produced polyester globally with an annual production exceeding 26 million tons for use in synthetic fibers, used for carpet, clothing, etc., and single-use beverage bottles, among others. Furthermore, current recycling technologies for PET typically focus on conversion via thermochemical or mechanical means, resulting in materials with similar but often reduced mechanical properties, and thus a lower value.

Biomass-derived chemical building blocks such as Muconate could be used to manufacture novel biopolymers such as fiber-reinforced plastic (FRP) with better mechanical properties than existing products. For biodegradable polymers, a microbial-based selective degradation strategy could be implemented to ensure 100% recirculation of bioplastic monomers, and those monomers could used to produce new polymer with the same properties as the virgin material, unlike thermo/chemically or mechanically recycled PET.

Currently, beverage manufacturing produces large amounts of biological wastes (tea alone about 500,000 metric tons), including used tea and ground coffee. These wastes are typically disposed in landfills or are used to produce composite materials such as paper, household utensils, and compost via a recycling process. Oxidative Hydrothermal Dissolution (OHD) is a novel conversion strategy for the efficient conversion of macromolecular solid organic materials, including lignocellulosic wastes, to low MW water-soluble organic products by reaction with small amounts of molecular oxygen in subcritical water. The process is simple and does not require a use of complex and expensive catalysts or solvents other than water.

However, there remains the need for better upcycling methods that can be used for converting plastics and biomass waste into high-value products, including chemical building blocks that can be then upcycled into new polymeric products.

### SUMMARY

This disclosure provides enzymatic compositions and methods for complete dissolution of macromolecular organic solid with the recovery of >90% of the initial carbon as dissolved products in some embodiments. The process is robust and widely applicable to a broad range of substrates, including used tea and coffee waste. In some embodiments, green or back tea waste may be converted to soluble organic monomers, which recombinant bacterial cell factories can efficiently use. In some embodiments, the substrate could be upcycled to high-value chemical building blocks such as 0-ketoadipate for the production of biodegradable plastic.

In one aspect, this disclosure provides a recombinant bacterial cell comprising a heterologous DNA encoding and expressing at least one heterologous PETase-like enzyme and at least one heterologous MHETase-like enzyme,
wherein the PETase-like enzyme has a secretion signal peptide linked in frame to an enzymatic activity for degrading Bis(2-hydroxyethyl) terephthalate (BHET) into Mono-(2-hydroxyethyl)terephthalic acid (MHET) and comprises Leaf-Compost Cutinase (LCC) enzyme with SEQ ID NO: 5, or a functional variant thereof having at least 85% overall sequence identity to SEQ ID NO: 5; and
wherein the MHETase-like enzyme has a secretion signal peptide linked in frame to an enzymatic activity for degrading MHET into ethylene glycol and terephthalic acid and comprises a polypeptide with SEQ ID NO: 6, a functional variant thereof having at least 85% overall sequence identity to SEQ ID NO: 6, Mle046 enzyme with SEQ ID NO: 13, a functional variant thereof having at least 85% overall sequence identity to SEQ ID NO: 13, Mle046 mutant enzyme with SEQ ID NO: 14, a functional variant thereof having at least 85% overall sequence identity to SEQ ID NO: 14, or any combination thereof.

Preferably, the recombinant bacterial cell may be Pseudomonas putida or Erwinia aphidicola. In some preferred embodiments, the PETase-like enzyme and/or the MHETase-like enzyme may be thermostable and enzymatically active at a temperature ranging from about 30° C. to about 75° C. and/or at a pH ranging from about 6 to about 9. In some embodiments, the PETase-like enzyme and/or the MHETase-like enzyme may be expressed from an inducible promoter. In some embodiments, the PETase-like enzyme and the MHETase-like enzyme may be encoded by a plasmid and co-expressed from a single promoter. In some embodiments, the recombinant bacterial cell may contain a mutation in its genome, the mutation eliminating expression of muconate cycloisomerase enzymatic activity with SEQ ID NO: 4, wherein the mutation is a deletion and/or insertion of at least one nucleotide or more. In some embodiments, the PETase-like enzyme and the MHETase-like enzyme are enzymatically active at 30° C., degrading bis(2-hydroxyethyl) terephthalate (BHET) into ethylene glycol and terephthalic acid. In some embodiments, the recombinant bacterial cell may be capable of growing on a substrate containing biomass co-mixed with polyethylene terephthalate (PET) pretreated in oxidative hydrothermal dissolution (OHD) process and prefereferrably, wherein the biomass contains green tea waste, black tea waste, used green tea, used black tea, corn stover and/or coffee brewing waste; and/or wherein the substrate contains the biomass and PET in the following ratio by weight from 1:99 wt % to 50:50 wt % of PET to the biomass.

In another aspect, this disclosure repates to a method for producing an enzymatic composition for biodegradation of plastic material and/or biomass waste, the method comprising:
culturing any of the recombinant bacterial cells accordingly to this disclosure in a liquid medium, wherein the recombinant bacterial cells secret the PETase-like enzyme and the MHETase enzyme into the liquid medium; and
collecting the liquid medium containing the PETase-like enzyme and the MHETase-like enzyme.

In some preferred embodiments of the method, the method may further comprise:
centrifuging a bacterial culture and producing a supernatant and a pellet; and
collecting the supernatant containing the PETase-like enzyme and the MHETase-like enzyme.

In yet another aspect, this disclosure repates to a method for decomposing a plastic material containing polyethylene terephthalate (PET) or poly(butylene adipate-co-terephthalate (PBAT), the method comprising: contacting the plastic material with the recombinant bacterial cell according to this disclosure and/or an enzymatic composition comprising at least one PETase-like enzyme and at least one MHETase-like enzyme, the enzymatic composition being produced or producible by the recombinant bacterial cell.

Some preferred embodiments of this method include those, wherein the method further comprises prior to contacting the plastic material with the recombinant bacterial cell, co-mixing the plastic material with a biomass and processing the co-mixture by oxidative hydrothermal dissolution (OHD) in the presence of oxygen in subcritical water at a temperature in the range 100-374° C. and a pressure in the range 1500 to 3500 psi. Preferably, the biomass and PET are present in a ratio by weight ranging from 1:99 wt % to 50:50 wt % of PET to the biomass. In some preferred embodiments of the method, the plastic material may be contacted at ambient temperature and a pH in the range from about 6 to about 9.

In yet another aspect, this disclosure relates to a method for converting a biomass into carbon-containing substrate for synthesizing polymeric products, the method comprising treating the biomass in a hydrothermal dissolution (OHD) process in the presence of oxygen in subcritical water at a temperature in the range 100-374° C. and a pressure in the range 1500 to 3500 psi, and contacting the OHD-treated biomass with a recombinant bacterium according to this disclosure, or an enzymatic composition comprising at least one PETase-like enzyme and at least one MHETase-like enzyme, the enzymatic composition being produced or being producible by the recombinant bacterial cell. In some preferred embodiments of the method, the biomass may include tea waste, coffee waste and/or corn stover.

In yet another aspect, this disclosure relates to a dual enzyme composition comprising at least one PETase-like enzyme and at least one MHETase-like enzyme, the composition being produced and secreted by the recombinant bacterial cell or being producible by the recombinant bacterial cell according to this disclosure. Preferably, the dual enzyme composition may comprise at least one PETase-like enzyme and at least one MHETase-like enzyme, wherein the PETase-like enzyme comprises at least amino acids 28-320 of the polypeptide with SEQ ID NO: 5 and/or a functional variant therefore having at least 80% overall sequence identity to amino acids 28-320 of SEQ ID NO: 5, or any combination thereof, wherein the PETase-like enzyme having an enzymatic activity for degrading bis(2-hydroxyethyl) terephthalate (BHET) to mono-(2-hydroxyethyl)terephthalic acid (MHET); and wherein the MHETase-like enzyme comprises at least amino acids 18-613 of the polypeptide with SEQ ID NO: 6 and/or a functional variant thereof having at least 80% overall sequence identity to amino acids 28-320 of SEQ ID NO: 5, or any combination thereof, and/or Mle046 enzyme comprising SEQ ID NO: 13 and/or a functional variant therefore having at least 80% overall sequence identity to SEQ ID NO: 13, and/or mutant Mle046 enzyme comprising SEQ ID NO: 14, or a functional variant thereof having at least 80% overall sequence identity to SEQ ID NO: 14, and wherein the MHETase-like enzyme has an enzymatic activity for degrading MHET to terephthalic acid (TPA) and ethylene glycol (EG). In some embodiments of the dual enzyme composition, at least one PETase-like enzyme and/or the at least one MHETase-like enzyme may be linked in frame with at least one signal peptide and/or at least one purification tag. Preferably, the molar ratio of the PETase-like enzyme to the MHETase-like enzyme may be in a range from about 1:99 to about 99:1.

In a further aspect, this disclosure relates to a method for producing cis-cis muconate, the method comprising contacting a substrate comprising terephthalic acid (TPA) with a recombinant bacterial cell containing a mutation in its genome, the mutation eliminating expression of muconate cycloisomerase enzymatic activity with SEQ ID NO: 4, wherein the mutation is a deletion and/or insertion of at least one nucleotide or more and wherein the substrate is produced by degradation of biomass waste and/or plastic material. Some preferred embodiments of the method include those, wherein the degradation includes an oxidative hydrothermal dissolution (OHD) process and/or reacting the substrate with the dual enzyme composition according to this disclosure.

### BRIEF DESCRIPTION OF THE DRAWINGS

FIG. 1 is a diagram showing the potent Erwinia aphidicola LJJL01 pathways being engineered to enable OHD-plastic derived Terephthalic acid (TPA). Green color genes indicated the heterologous genes (Comamonas sp. E6).

FIG. 2 is a diagram showing the developed integrated process of OHD and engineered monoculture to funnel heterogeneous OHD products (aromatics, sugars, acids) to a single product (i.e. beta-ketoadipate). (˜90% yield from OHD aromatics)

FIG. 3 is a series of pictures and diagrams showing the efficient protein secretion in E. aphidicola LJJL01LJJL01 by employing I. sakaiensis secretion peptides.

FIG. 4 is a series of graphs and diagrams showing the selective degradation of BHET by engineered E. aphidicola LJJL01.

FIG. 5 is a series of graphs and diagrams showing the unspecific degradation of BHET and expression limitation of LCC in P. putida KT2240. The results are expressed as means±SEM (n=3).

FIG. 6 is a series of graphs and diagrams showing the synergistic effect and selectivity of BHET degradation of engineered E. aphidicola LJJL01 with LCC and MHETase enzymes. The results are expressed as means±SEM (n=3).

FIG. 7 is a graph and picture set showing the expression and secretion of fungal PU degradation enzymes in E. aphidicola LJJL01. The results are expressed as means±SEM (n=3).

FIG. 8 is a diagram showing the plasmid map of the LCC and MHETase.

FIG. 9 is a diagram showing a plasmid map of the expression of fungal PU-degradation genes in E. aphidicola LJJL01 design.

FIG. 10 is a diagram showing an additional plasmid map of the expression of fungal PU-degradation genes in E. aphidicola LJJL01 design.

FIG. 11 is a diagram showing the developed in situ degradation of PET without chemical catalyst of external supplementation of chemical (diol/acids), and the integrated OHD and engineered microbes to recover the original plastic monomer (i.e., TPA).

FIG. 12 is a series of graphs showing the conversion of OHD-plastic and corn stover-derived products by P. putida KT2440.

FIG. 13 is a series of graphs and a diagram showing the employment of PET-degradation enzymes to recover TPA from OHD-plastic.

FIG. 14 is a diagram showing the potential PET upcycling routes that can be engineered in E. aphidicola LJJL01.

FIG. 15 is a diagram showing the selectively designed microbe for depolymerization of PET.

FIG. 16 reports synergistic activity of Mle046 or Mle046 mutant with LCC. TPA (rhombus), MHET (triangle), and BHET (rectangle) concentrations (mM) along with OD₆₀₀ mapped out over 72 hours for EA-pBLT2, EA-LCC, EA-LCC-Mle046, and EA-LCC-Mle046 (mutant) at pH 7 and pH 8.

FIG. 17 reports TPA and MHET (g/h) produced by EA-pBLT2, EA-LCC, EA-LCC-Mle046, and EA-LCC-Mle046 (mutant) at pH 7 and pH 8. Results are given as the average of n=3 with the corresponding SEM. Bars labeled with different letters indicate statistical significance (p<0.05; one-way ANOVA followed by Tukey's post hoc honest significance difference test).

FIG. 18 reports MHET degradation by EA-pBLT2, EA-LCC, EA-LCC-Mle046 and EA-LCC-Mle046 (mutant). Results are given as the average of n=3 with the corresponding SEM. Bars labeled with different letters indicate statistical significance (p<0.05; one-way ANOVA followed by Tukey's post hoc honest significance difference test).

FIG. 19 reports TPA production by EA-pBLT2, EA-LCC, EA-LCC-Mle046, and EA-LCC-Mle046 (mutant). Results are given as the average of n=3 with the corresponding SEM. Bars labeled with different letters indicate statistical significance (p<0.05; one-way ANOVA followed by Tukey's post hoc honest significance difference test).

FIG. 20 is a plasmid map for pBLT-2-LCC-Mle046 construct for co-expressing LCC and Mle046 in bacterial cells.

FIG. 21 is a restriction map of a portion of the pBLT-2-LCC-mutant Mle046 construct.

### DETAILED DESCRIPTION

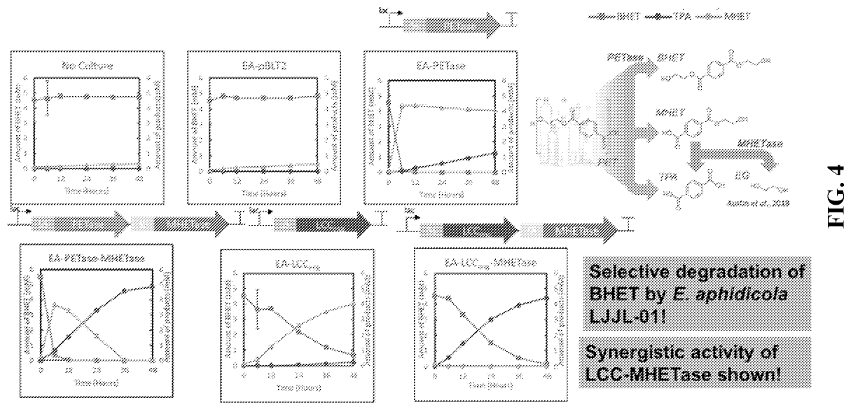

The following abbreviations may be used in this disclosure:
“MW” means molecular weight;
“PET” means polyethylene terephthalate, with its chemical structure being shown in FIG. 4;
“PBAT” means poly(butylene adipate-co-terephthalate);
“BHET” means bis(2-hydroxyethyl) terephthalate, with its chemical structure being shown in FIG. 4;
“MHET” means mono-(2-hydroxyethyl)terephthalic acid, with its chemical structure being shown in FIG. 4;
“TPA” means terephthalic acid which can be also referred to as benzene-1,4-dicarboxylic acid, with its chemical structure being shown in FIG. 4;
“EG” means ethylene glycol, with its chemical structure being shown in FIG. 4;
“OHD” means oxidative hydrothermal dissolution process;
“An OHD-substrate” means a substrate that was pretreated by the OHD process;
“LCC” means leaf-compost cutinase;
“PETase-like enzyme” means an enzyme having an enzymatic activity for degrading BHET into MHET; and
“MHETase-like enzyme” means an enzyme having an enzymatic activity for degrading MHET into TPA and EG.

In this disclosure the term “microbe” means a microbial organism and may refer to a bacterium, including a recombinant bacterium comprising heterologous DNA, and/or a recombinant bacterium produced by various genetic manipulations, including those that may result in a bacterial gene being overexpressed, knocked out or knocked down.

In this disclosure, the term “secreted protein” means a protein that a bacterial cell secretes from its cytoplasm through its cytoplasmic membrane and preferably also through the bacterial wall after the protein has been synthetized in the cytoplasm, as shown for example in FIG. 3. A secreted protein comprises at least one secretion signal peptide, which can be referred to as “a signal peptide,” and which is necessary for secretion and is recognized by the bacterial secretion system, for example as shown in FIG. 3. This signal peptide may be cleaved off by the bacterial secretion system, as shown for example in FIG. 3.

In this disclosure, the term “about” means±5% of the value. For example, “about 100” means a range from 95 to 105 and “about 200” means a range from 190 to 210.

In this disclosure, the term “ambient temperature” means a temperature in the range from about 10° C. to about 35° C.

In this disclosure, “heterologous DNA” means DNA that originates from a source other than a host bacterium species, including, but not limited to, synthetic DNA molecules produced in a laboratory and/or DNA encoding gene(s) from another microbial organism and/or plasmids. The terms “heterologous DNA sequence”, “exogenous DNA segment” or “heterologous nucleic acid,” as used herein, each refer to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. A “homologous” DNA sequence is a DNA sequence that is naturally associated with a host cell into which it is introduced.

Upcycling includes various processes for creating new products from waste plastic or biomass materials. Upcycling not only reduces waste, it also converts waste into useful products. This disclosure provides biocatalysts and methods for energy-efficient and environmentally safe upcycling of waste biomass and/or plastic materials, including post-consumer PET, a synthetic polymer which is commonly difficult to biodegrade. The biocatalysts and methods disclosed herein not only degrade biomass and/or plastic waste efficiently, they convert the waste into organic monomers with an important commercial value for producing new polymeric materials.

PET is difficult to hydrolyze due to its high crystallinity, which reduces chain mobility and prevents substrate-enzyme binding. According to published methods, PET must be heated to about 72° C. to reach the glass transition temperature, the point at which PET will exhibit macromolecular mobility. This allows it to fit more comfortably into the PHEs active site to be cleaved.¹ Previously, leaf-compost cutinase (LCC) has been engineered to degrade about 90% of postconsumer-PET (pc-PET) pretreated for 10 h at 72° C. and at a pH of 8.0.1 This produces 16.7 g of TPA L⁻¹ h⁻¹ with an enzyme concentration of 3 mg of LCC per 1 g of PET.¹ This enzyme degrades PET into MHET, but is limited by the low rate at which LCC degrades MHET into ethylene glycol and terephthalic acid (TPA)¹. Yet, TPA has a much higher upcycling value and can also be used for production of other commercially valuable monomers such as muconate.

In one aspect, this disclosure provides a dual enzyme composition (biocatalyst) for efficient decomposition of a biomass waste, a post-consumer plastic material, preferably the plastic material containing PET and/or PBAT, or any combination thereof. The dual enzyme composition comprises at least one PETase-like enzyme and at least one MHETase-like enzyme, the dual enzyme composition being produced and secreted by a recombinant bacterial cell expressing the enzymes from heterologous DNA. Preferred recombinant bacteria include, but are not limited to, Pseudomonas putida or Erwinia aphidicola. A particularly preferred bacterium is Erwinia aphidicola which is a Gram-negative, oxidase-negative, facultatively anaerobic, fermentative, rod-shaped bacterium as was described by Harada and co-workers in J. Gen Appl Microbiol., 1997, December; 43(6):349-354.

The inventors unexpectedly found that the combination of the two enzymes has a synergistic effect for degrading plastic materials and in particular plastic materials such as BHET which is an intermediary compound in PET degradation. It was also unexpectedly found that co-mixing PET with biomass waste improves the degradation rate of PET, allowing for conducting its depolymerization reaction at a lower temperature, preferably lower than 70° C., and more preferably at an ambient temperature at least in some embodiments wherein PET and biomass co-mixture was pre-treated in the OHD process.

In some preferred embodiments, the PETase-like enzyme may include, but is not limited to Leaf-Compost Cutinase (LCC) enzyme comprising, consisting essentially of, or consisting of a polypeptide with SEQ ID NO: 5 and/or a functional variant thereof having at least 80, 81, 82, 83, 84, 85, 86, 87, 88, 90, 91, 92, 93, 94, 95, 96, 97, 98, or 99% overall sequence identity to SEQ ID NO: 5, or any combination thereof. The PETase-like enzyme has an enzymatic activity for degrading BHET to MHET.

In some preferred embodiments, the PETase-like enzyme may include, but is not limited to a LCC functional fragment comprising, consisting essentially of, or consisting of amino acids 28-320 of the polypeptide with SEQ ID NO: 5, and preferably it does not include amino acids 1-27 of the polypeptide with SEQ ID NO: 5. In some preferred embodiments, the PETase-like enzyme may include a functional variant having at least 80, 81, 82, 83, 84, 85, 86, 87, 88, 90, 91, 92, 93, 94, 95, 96, 97, 98, or 99% overall sequence identity to amino acids 28-320 of SEQ ID NO: 5, or any combination thereof. The PETase-like enzyme has an enzymatic activity at least for degrading BHET to MHET.

The dual enzyme compositions (biocatalysts) according to this disclosure may comprise any of the above PETase-like enzymes and one or more MHETase-like enzymes. In some preferred embodiments, the MHETase-like enzyme may comprise, consist essentially of, or consist of a polypeptide with SEQ ID NO: 6 and/or a functional variant thereof having at least 80, 81, 82, 83, 84, 85, 86, 87, 88, 90, 91, 92, 93, 94, 95, 96, 97, 98, or 99% overall sequence identity to SEQ ID NO: 6, or any combination thereof. The MHETase-like enzyme has an enzymatic activity for degrading MHET to TPA and EG.

In some preferred embodiments, the MHETase-like enzyme may include, but is not limited to a MHETase-like functional fragment comprising, consisting essentially of, or consisting of amino acids 18-613 of the polypeptide with SEQ ID NO: 6, and preferably it does not include amino acids 1-17 of the polypeptide with SEQ ID NO: 6. In some preferred embodiments, the MHETase-like enzyme may include a functional variant having at least 80, 81, 82, 83, 84, 85, 86, 87, 88, 90, 91, 92, 93, 94, 95, 96, 97, 98, or 99% overall sequence identity to amino acids 18-613 of SEQ ID NO: 6, or any combination thereof. The MHETase-like enzyme has an enzymatic activity at least for degrading MHET to TPA and EG.

Other suitable MHETase-like enzymes include, but are not limited to, Mle046 enzyme comprising, consisting essentially of, or consisting of SEQ ID NO: 13, and/or a functional variant thereof having at least 80, 81, 82, 83, 84, 85, 86, 87, 88, 90, 91, 92, 93, 94, 95, 96, 97, 98, or 99% overall sequence identity to SEQ ID NO: 13, or any combination thereof. The MHETase-like enzyme has an enzymatic activity for degrading MHET to TPA and EG.

Other suitable MHETase-like enzymes further include, but are not limited to, mutant Mle046 enzyme comprising, consisting essentially of, or consisting of SEQ ID NO: 14, and/or a functional variant thereof having at least 80, 81, 82, 83, 84, 85, 86, 87, 88, 90, 91, 92, 93, 94, 95, 96, 97, 98, or 99% overall sequence identity to SEQ ID NO: 14, or any combination thereof. The MHETase-like enzyme has an enzymatic activity at least for degrading MHET to TPA and EG.

Mle046, is a marine MHETase enzyme identified through a metagenomics study, a homolog of MHETase of the PET-degrading bacterium Ideonella sakaiensis.² Mle046 enzyme degrades MHET into ethylene glycol and terephthalic acid efficiently, and is active in the temperature range of 10 to 60° C.², and at pH levels of 6.5-9.0.2 The primary reason for the interest in Mle046 is the turnover rate (K_{cat}) of MHET degradation by Mle046 is much higher than other MHETases.² What limits this enzyme is the enzyme-substrate affinity represented by Kₘ. Because Mle046 has a higher Kₘ, it reduces the catalytic efficiency by nearly 40 times compared to the similar MHETase enzymes. Unexpectedly, and as is reported in detail below, mutant Mle046 (Gi 17S) with SEQ ID NO. 14 has an improved conversion rate for producing TPA from MHET. Advantageously, the dual enzyme composition has a high substrate conversion rate at ambient temperature over a broad range of pH, including from about 6 to about 9.

It should be understood that any of the PETase-like and/or MHET-like enzymes according to this disclosure may further comprise one or more purification tags and/or one or more signal peptides in order to simplify enzyme secretion from a cell, and in particular from a bacterial cell, and/or purification from cell culture medium, if necessary. Examples of purification tags include, but are not limited to, His-tag or c-Myc tag, preferably linked at the C-terminus of the enzyme. Examples of signal peptides include, but are not limited to, MNFPRASRLMQAAVLGGLMA (SEQ ID NO: 44) and VSAAATAMQTTVTTMLLASVALAA (SEQ ID NO: 45), preferably linked to the N-terminus of the enzyme in frame with the enzyme, for example as shown in FIG. 3.

In this disclosure “a functional variant” means a polypeptide that may perform the same enzymatic reaction as its corresponding enzyme. Some functional variants may contain one or more of amino acid substitutions which do not significantly impact the enzymatic function. For example, one negatively charged amino acid may be substituted for another negatively charged amino acid. Some other examples include, but are not limited to, one or more amino acid deletion and/or insertion, especially when such mutations are made outside the enzyme catalytic domain.

In the dual enzyme compositions (biocatalysts) according to this disclosure, one or more PETase-like enzymes and one or more MHETase-like enzymes may be present in any molar ratio. Preferably, the molar ratio of the PETase-like enzyme to the MHETase-like enzyme may be in a range from about 1:99 to about 99:1.

One of the technical advantages for the dual enzyme compositions according to this disclosure is that they are thermostable and enzymatically active at a broad range of temperatures, including from about 10° C. to about 90° C., and most preferably from about 30° C. to about 75° C. Another technical advantage of the dual enzyme compositions according to this disclosure is that they remain enzymatically active in a broad range of pH, preferably in the pH range from about 6.0 to about 9.0. While prior art enzymes for degrading PET typically require an elevated temperature, e.g., at least about 70° C., the present compositions are suitable for upcycling PET and/or biomass waste at a much lower temperature, such as for example, at ambient temperature, including in the range from about 20° C. to about 35° C., and most preferably, from about 25° C. to about 30° C.

The dual enzyme compositions according to this disclosure are suitable for producing TPA and EG from a great variety of different substrates. Suitable substrates include, but are not limited to, biomass waste, post-consumer plastic, in particular PET and PBAT, or any combination thereof.

In this disclosure the term “biomass” refers to biological material derived from plants, fungi, microbial organisms or animal-derived wastes. Biomass waste refers to any biomass that is typically discarded for example, by burning, burial or other disposal methods.

Examples of suitable biomass substrates include, but are not limited to, algae; grass; wood; tree; shrub; tree leaves; tree needles; bushes; agricultural biomass wastes including leaves, crop stalks, roots, fruit and/or vegetable skins, corn stover, rice hulls, grain husks; beverage industry waste including black tea waste, green tea waste, ground coffee; forestry wood waste including branches, trees, bushes; construction and demolition biomass waste including saw dust, scrap wood; post-consumer biomass waste including paper, cardboard, used tea leaves, used ground coffee, peeled vegetable or fruit skins and other food preparation wastes. Preferred examples of biomass wastes include, but are not limited to, green tea waste, black tea waste, used tea leaves, ground coffee and/or corn stover.

Preferred substrates for the dual enzyme composition according to this disclosure include those which are pre-treated by oxidative hydrothermal dissolution (OHD) process in the presence of oxygen in subcritical water in a reactor at an elevated temperature under pressure. This pre-treated substrate may be referred in this disclosure as OHD-substrate, e.g., OHD-biomass and/or OHD-plastic. In order to OHD-pretreat the substrate, biomass and/or PET containing plastic material may be subjected to an OHD process which may be conducted at a temperature in the range from about 100° C. to about 374° C., and preferably in the range from about 200° C. to about 350° C. The pressure in the reactor may be specified to at least maintain the water in liquid state. In some embodiments, the pressure may be in the range 1500 to 3500 psi. “Subcritical water” means high-temperature and high-pressure water. Examples of OHD methods are known in the art, for example from U.S. Pat. No. 10,023,512, the entire disclosure of which is herein incorporated by reference.

Preferably, the dual enzyme composition according to this disclosure is produced in a recombinant bacterial cell which contains heterogenous DNA, e.g., a plasmid, encoding and expressing at least one the PETase-like enzyme and at least one the MHETase-like enzyme.

Thus, in another aspect, this disclosure relates to a recombinant bacterial cell comprising a heterologous DNA encoding and expressing at least one heterologous PETase-like enzyme and at least one heterologous MHET enzyme. Preferably, the bacterial cell is Pseudomonas putida or Erwinia aphidicola.

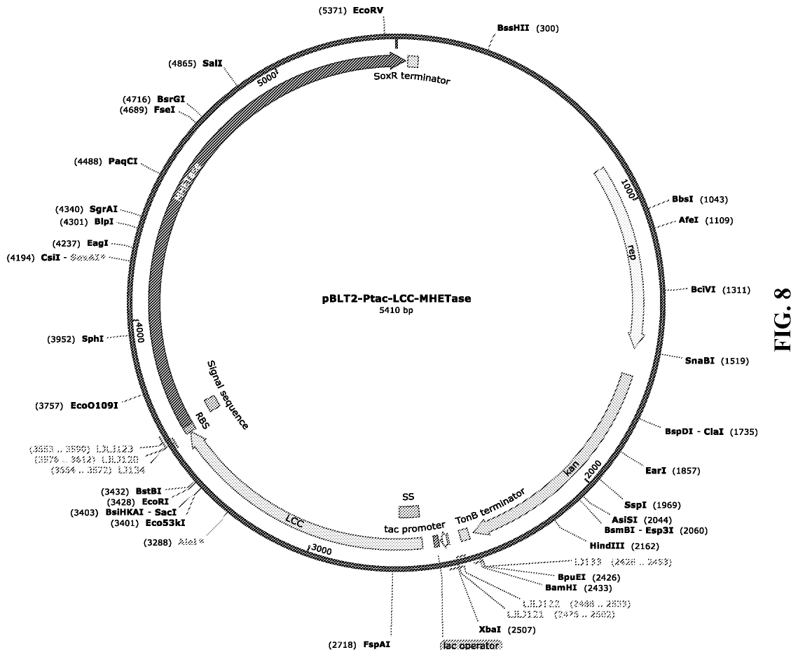

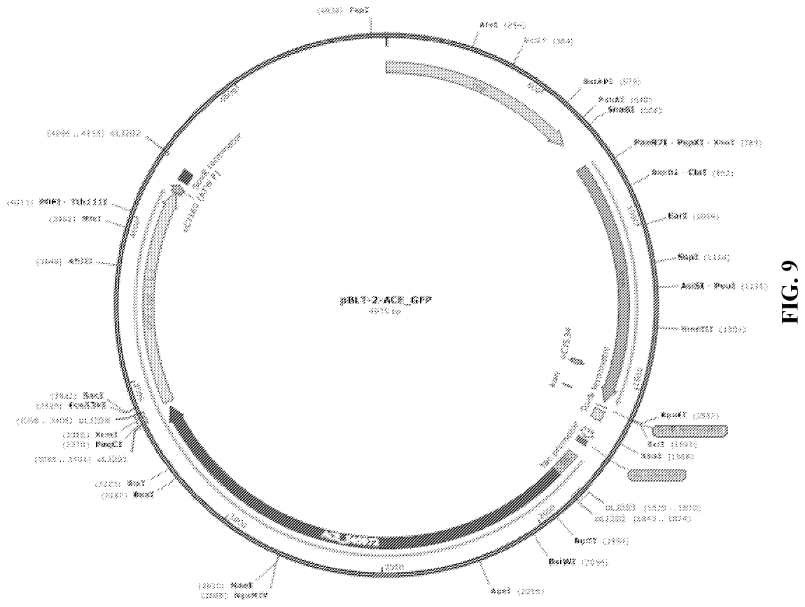

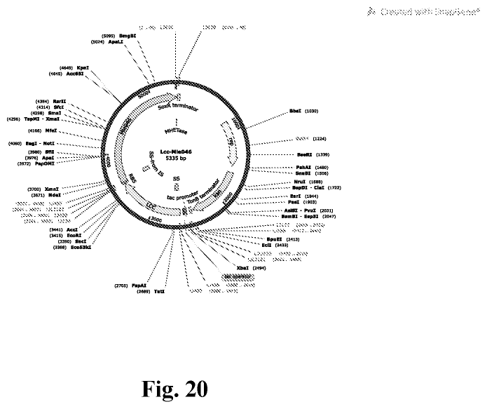

Preferably, at least one the PETase-like enzyme and at least one the MHETase-like enzyme are encoded by a plasmid, some examples of which are shown in plasmid maps of FIGS. 8, 9, 10, and 20. In some embodiments, for example as shown in FIG. 20, a plasmid may express both enzymes from the same promoter. Preferably, since each of the enzymes is produced with at least one signal peptide, each of the two enzymes is secreted from the recombinant bacterial cell. In some preferred embodiments, the recombinant cell may comprise one or more of the following heterologous DNA genes: SEQ ID NO: 7 encoding the LCC enzyme and MHETase enzyme, SEQ ID NO: 8 encoding LCC enzyme, SEQ ID NO: 11 encoding Mle046 enzyme, SEQ ID NO: 12 encoding mutant Mle046 enzyme, or any variants thereof having at least 80, 81, 82, 83, 84, 85, 86, 87, 88, 90, 91, 92, 93, 94, 95, 96, 97, 98, or 99% overall sequence identity to SEQ ID NO: 7, 8, 11, or 12, or any combination thereof.

A recombinant bacterial cell according to this disclosure may express and secrete a combination of a least one PETase-like enzyme according to this disclosure and at least one MHETase-like enzyme according to this disclosure. In some embodiments, a recombinant bacterial cell may comprise a heterologous DNA encoding and expressing at least one heterologous PETase-like enzyme and at least one heterologous MHETase-like enzyme, wherein the PETase-like enzyme has a secretion signal peptide linked in frame to an enzymatic activity for degrading Bis(2-hydroxyethyl) terephthalate (BHET) into Mono-(2-hydroxyethyl)terephthalic acid (MHET) and comprises Leaf-Compost Cutinase (LCC) enzyme with SEQ ID NO: 5, a functional variant thereof having at least 85% overall sequence identity to SEQ ID NO: 5, or any combination thereof, and wherein the MHETase-like enzyme has a secretion signal peptide linked in frame to an enzymatic activity for degrading MHET into ethylene glycol and terephthalic acid and comprises a polypeptide with SEQ ID NO: 6, a functional variant thereof having at least 85% overall sequence identity to SEQ ID NO: 6, Mle046 enzyme with SEQ ID NO: 13, a functional variant thereof having at least 85% overall sequence identity to SEQ ID NO: 13, Mle046 mutant enzyme with SEQ ID NO: 14, a functional variant thereof having at least 85% overall sequence identity to SEQ ID NO: 14, or any combination thereof.

In some preferred embodiments, the recombinant bacterial cell according to this disclosure further comprises a mutation in its genome, the mutation eliminating expression of muconate cycloisomerase activity with SEQ ID NO: 4, wherein the mutation is a deletion and/or insertion of at least one nucleotide or more. This mutation results in accumulation of cis-cis muconate in the recombinant bacterial cell, which is a highly valuable monomer in production of plastic materials. In some preferred embodiments, the mutation can be achieved by knocking out a gene encoding muconate cycloisomerase with SEQ ID NO: 4, preferably having a nucleotide sequence with SEQ ID NO. 3. Particularly preferred mutagenesis methods include genome editing methods, one example of which is CRISPR-Cas9 based-gene deletion as described in detail in Example 3.

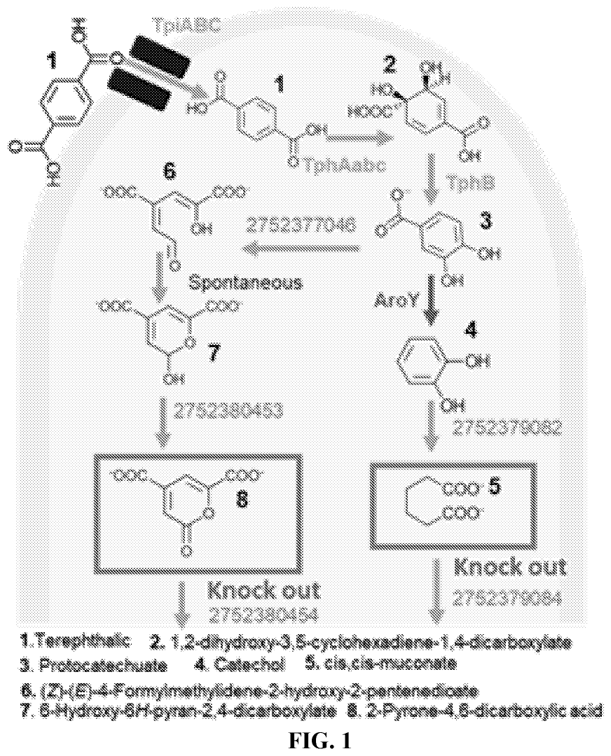

Technical advantages of recombinant bacterial cells according to this disclosure include the capacity of the recombinant bacterial cells to grow on substrates that contain oxidative hydrothermal dissolution (OHD) processed substrate, which may include biomass waste, PET plastic, or any mixture thereof. It has been unexpectedly found that growing the bacterial cells in the presence of the OHD-pretreated biomass and/or OHD-pretreated PET substrate results in efficient conversion of the OHD-pretreated biomass and/or PET to TPA and EG. Advantageously in the embodiments wherein the recombinant bacterial cells further comprise a deleted muconate cycloisomerase activity, the recombinant bacterial cells may further effectively intake TPA into the cell and process TPA to cis,cis-muconate as shown for example in FIG. 1. In some embodiments, the substrate may contain the OHD-pretreated mixture of biomass and PET in the following ratio by weight from 1:99 wt % to 50:50 wt % of PET to the biomass.

In yet another aspect, this disclosure relates to a method for producing an enzymatic composition (biocatalyst) for biodegradation of a plastic material and/or biomass waste, the method comprising culturing any of the recombinant cells according to this disclosure which produce and secrete a combination of at least one PETAse-like enzyme according to this disclosure and at least one MHETase-like enzyme according to this disclosure in a liquid medium, wherein the recombinant bacterial cells secret the PETase-like enzyme and the MHETase enzyme into the liquid medium; and collecting the liquid medium containing the PETase-like enzyme and the MHETase-like enzyme. It should be noted that the cells can be grown for a period of time in suspension, for example from about 10 hours to about 48 hours and even longer if necessary. The cells may be grown at any temperature suitable for bacterial growth, for example at a temperature in the range from about 30° C. to about 37° C., and preferably with agitation in order to improve access of oxygen to the cells. The liquid medium may contain M9 minimal medium containing M9 minimal medium (Fisher Scientific) containing 33.9 g/L disodium phosphate (anhydrous), 15.0 g/L monopotassium phosphate, 2.5 g/L sodium chloride, 5.0 g/L ammonium chloride, 4 mM magnesium sulphate, 36 μM ferrous sulphate, 200 μM calcium chloride supplemented with 20 mM glucose (Fisher Scientific). Other liquid growth medium typically used for growing bacterial cells may be also used. The bacterial growth in suspension can be monitored by measuring periodically an optical density (O.D.) of the bacterial culture at 600 nm in a spectrophotomer.

In some embodiments, a substrate containing OHD-pretreated PET and/or OHD-pretreated biomass waste may be added directly to the liquid growth medium. For example, such substrates may be added to the total concentration in the liquid growth medium of 1 mM to 10 mM.

In some embodiments, the cells may be allowed to grow for a period of time without the substrate being added, the liquid growth medium may be then reacted with the substrate. It should be further noted that it is not necessary to separate the cells from the growth medium which contains the enzymatic composition. However, in some embodiments, tie growth medium containing the enzymatic composition may be separated from the cells by any conventional method, including, but not limited to, centrifugation and/or filtration. In some embodiments, the growth medium containing the enzymatic composition may be further processed by being subjected to any of methods typically used for purification of enzymes, including, but not limited to, using chromatography, centrifugation, protein precipitation and/or gel purification.

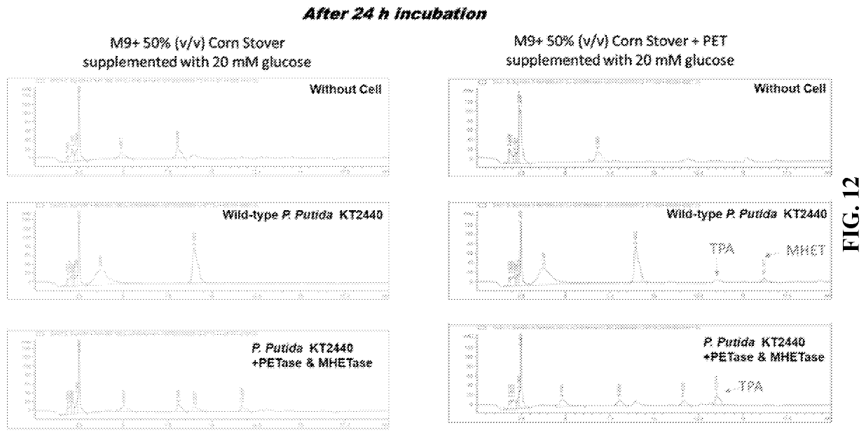

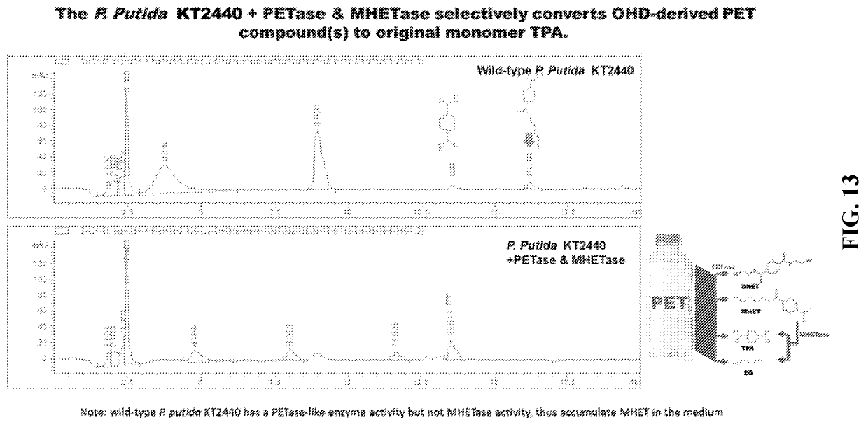

In yet further aspect, this disclosure relates to methods for decomposing a plastic material comprising PET and/or PBAT, the method comprising contacting the material with the recombinant bacterial cells according to this disclosure which express and secrete at least one PETase-like enzyme and at least one MHETase-like enzyme according to this disclosure. In the alternative, the plastic material may be contacted with the culture growth medium containing at least one PETase-like enzyme and at least one MHETase-like enzyme produced by the cells and or with a dual enzyme composition according to this disclosure. Some preferred embodiments of this method may be conducted at ambient temperature and a pH ranging from about 6 to about 9. The reaction can be carried out for a period of time, for example for any period of time from about 10 minutes to about 48 hours, or shorter, or longer as may be needed. A conversion rate and production of TPA and EG may be monitored for example, as discussed in examples in connection with FIGS. 12 and 13. A ratio of a substrate to the enzymes may be adjusted as needed. In some embodiments, from about 0.5 mg to about 5 mg of each of the two enzymes may be used per one gram of the substrate.

In some preferred embodiments of the method, prior to contacting the plastic material, preferably the plastic material comprising PET or PBAT, with the recombinant bacterial cell or the dual enzyme composition produced or producible by the recombinant bacterial cell, the plastic material may be co-mixed with a biomass. Preferably, the co-mixture, or the plastic material itself, is subjected to oxidative hydrothermal dissolution (OHD) process in the presence of oxygen in subcritical water at a temperature in the range 220-300° C. and a pressure in the range 1500 to 3500 psi, prior to be contacted with the recombinant bacterial cells or the dual enzyme composition. It has been unexpectedly found that co-mixing a plastic material, and in particular a PET containing plastic material with biomass, may improve the conversion rate of PET decomposition and in particular lead to the possibility of performing the degradation reaction at a temperature lower, e.g., lower than 70° C., than what is typically needed for decomposition of PET.

In some preferred embodiments for co-mixtures, the biomass and the plastic material, preferably PET and/or PBAT, may be present in the ratio by weight ranging from 1:99 wt % to 50:50 wt % of PET to the biomass.

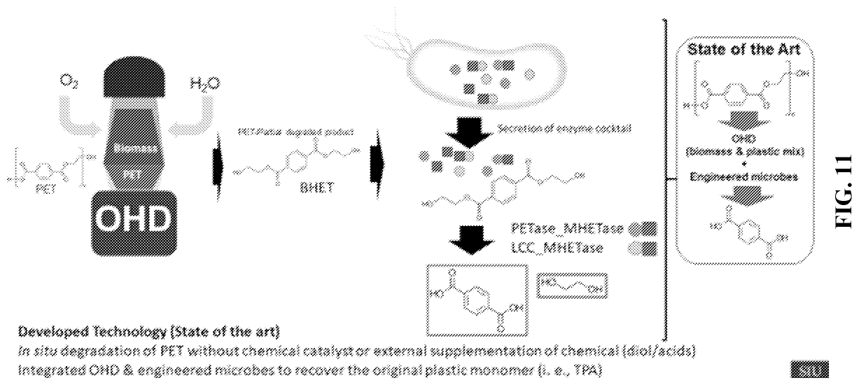

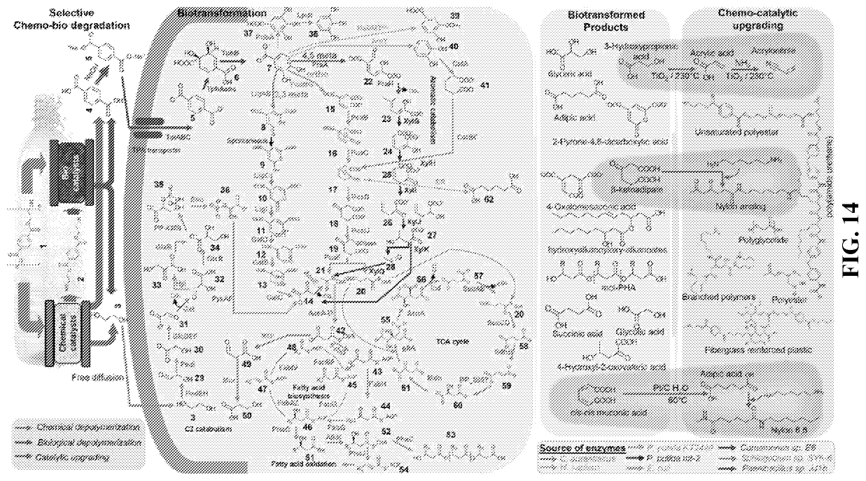

It has been unexpectedly found that the dual enzyme composition according to this disclosure has a synergistic property with respect to degrading PET. With reference to FIG. 11, it has been unexpectedly found that the present methods permit for in situ degradation of PET without a chemical catalyst or external supplementation of corrosive chemicals such as for example as diols and acids. Present methods which integrate the OHD process and degradation by the recombinant bacterial cells recover the original plastic monomer (TPA) which is highly valuable in production of new polymeric materials, including nylon and other materials as shown for example in FIG. 14.

One embodiment of the present disclosure is an engineered recombinant cell for conversion of a waste biomass feedstock into a chemical feedstock. The engineered microbe may be P. putida KT2440, E. aphidicola LJJL01, or any other suitable microbe including yeast (S. cerevisiae). The engineered microbe may comprise a deletion of a polynucleotide encoding a polypeptide with muconate cycloisomerase activity. The polynucleotide encoding a polypeptide with muconate cycloisomerase activity may comprise SEQ ID NO. 3, or a sequence at least 95% identical thereto, or a full-length complement thereof, or a functional fragment thereof. Alternatively, the polypeptide with muconate cycloisomerase activity may comprise SEQ ID NO. 4, or a sequence at least 95% identical thereto, or a full-length complement thereof, or a functional fragment thereof.

The waste biomass feedstock may comprise an OHD end product stream. The OHD end product stream may be generated by performing the OHD process on an OHD input stream comprising green tea, black tea, coffee, corn stover, coal, any other biomass material suitable for the OHD process, or a mixture thereof.

The chemical feedstock may comprise terephthalic acid, terephthalate, 1,2-dihydroxy-3,5-cyclohexadiene-1,4-dicarboxylate, protocatechuate, catechol, muconate, (z)-(e_-4-formylmethylidene-2-hydroxy-2-pentadioate, 6-hydroxy-6H-pyran-1,4-dicarboxylate, 2-pyrone-4,6-dicaboxylic acid, caffeine, vanillic acid, ferulic acid, coumaric acid, and syringic acid. Preferably, the chemical feedstock may comprise muconate, including cis,cis-muconate, beta-ketoadipate, or a mixture of the two.

Embodiments of this disclosure include OHD upcycling of plastics with engineered microbes. Another embodiment of the present disclosure is an engineered microbe for conversion of a waste plastic feedstock into a chemical feedstock. The engineered microbe (recombinant bacterial cell) may be P. putida KT2440, E. aphidicola LJJL01, or any other suitable microbe.

The engineered microbe may comprise an exogenous (heterologous) polynucleotide encoding a polypeptide with LCC enzyme activity. The polypeptide with LCC enzyme activity may comprise SEQ ID NO. 5, or a sequence at least 95% identical thereto, or a full-length complement thereof, or a functional fragment thereof.

The engineered microbe may comprise an exogenous polynucleotide encoding a polypeptide with MHET enzyme activity. The polypeptide with MHET enzyme activity may comprise SEQ ID NO. 6, or a sequence at least 95% identical thereto, or a full-length complement thereof, or a functional fragment thereof.

The engineered microbe may comprise an exogenous polynucleotide encoding a polypeptide with LCC enzyme activity and an exogenous polynucleotide encoding a polypeptide with MHET enzyme activity. The exogenous polynucleotide encoding a polypeptide with LCC enzyme activity and the exogenous polynucleotide encoding a polypeptide with MHET enzyme activity may be as described above. Alternatively, the exogenous polynucleotide encoding a polypeptide with LCC enzyme activity and the exogenous polynucleotide encoding a polypeptide with MHET enzyme activity may comprise a polynucleotide encoding a fusion protein with dual LCC enzyme activity and MHET enzyme activity.

The waste plastic feedstock may comprise an OHD end product stream. The OHD end product stream may be generated by performing the OHD process on an OHD input stream comprising PET, green tea, black tea, coffee, corn stover, coal, any other biomass material suitable for the OHD process, or a mixture thereof.

The chemical feedstock may comprise Bis(2-hydroxyethyl) terephthalate (BHET), Mono-(2-hydroxyethyl)terephthalic acid (MHET), Terephthalic acid (TPA), EG, or a mixture of the two.

### Sequences and Mutations

The amino acid sequences and nucleic acid sequences described herein may contain various mutations. Mutations may include insertions, substitutions, and deletions. Insertions are written as follows: (+)(amino acid/nucleic acid sequence position number)(inserted amino acid/nucleic acid base). For example, +287A would mean an insertion of an alanine residue after position 287 in the corresponding amino acid sequence. Substitutions are written as follows: (amino acid/nucleic acid base to be replaced)(amino acid/nucleic acid sequence position number)(substituted amino acid/nucleic acid base). For example, C1082A would mean a substitution of an adenine base instead of a cytosine base at position 1082 in the corresponding nucleic acid sequence. Deletions are written as follows: (amino acid/nucleic acid base to be deleted)(amino acid/nucleic acid sequence position number)(−). For example, C970—would mean a deletion of the cytosine base normally located at position 970 in the corresponding nucleic acid sequence.

The amino acid sequences and nucleic acid sequences described herein may contain mutations at various sequence positions. Sequence positions may be written a variety a ways for convenience. More specifically, sequence positions may be written from either the beginning of the sequence as a positive position number, or from the end of the sequence as a negative number. Sequence positions may be converted easily between a positive notation and a negative notation by comparing to the sequence length and either adding or subtracting the sequence length. For example, a promoter containing 10 nucleic acid bases with a mutation from cytosine to adenine at the second position from the start of the sequence may be written as C2A. Alternatively, this mutation may be written as C(−9)A, −9C/A, or in a similar fashion denoting the negative position number.

The following definitions and methods are provided to better define the present invention and to guide those of ordinary skill in the art in the practice of the present invention. Unless otherwise noted, terms are to be understood according to conventional usage by those of ordinary skill in the relevant art.

An “allele” refers to one of two or more alternative forms of a genomic sequence at a given locus on a chromosome.

The term “chimeric” is understood to refer to the product of the fusion of portions of two or more different polynucleotide molecules. “Chimeric promoter” is understood to refer to a promoter produced through the manipulation of known promoters or other polynucleotide molecules. Such chimeric promoters can combine enhancer domains that can confer or modulate gene expression from one or more promoters or regulatory elements, for example, by fusing a heterologous enhancer domain from a first promoter to a second promoter with its own partial or complete regulatory elements. Thus, the design, construction, and use of chimeric promoters according to the methods disclosed herein for modulating the expression of operably linked polynucleotide sequences are encompassed by the present disclosure.

Novel chimeric promoters can be designed or engineered by a number of methods. For example, a chimeric promoter may be produced by fusing an enhancer domain from a first promoter to a second promoter. The resultant chimeric promoter may have novel expression properties relative to the first or second promoters. Novel chimeric promoters can be constructed such that the enhancer domain from a first promoter is fused at the 5′ end, at the 3′ end, or at any position internal to the second promoter.

A “construct” is generally understood as any recombinant nucleic acid molecule such as a plasmid, cosmid, virus, autonomously replicating nucleic acid molecule, phage, or linear or circular single-stranded or double-stranded DNA or RNA nucleic acid molecule, derived from any source, capable of genomic integration or autonomous replication, comprising a nucleic acid molecule where one or more nucleic acid molecule has been operably linked.

A construct of the present disclosure can contain a promoter operably linked to a transcribable nucleic acid molecule operably linked to a 3′ transcription termination nucleic acid molecule. In addition, constructs can include but are not limited to additional regulatory nucleic acid molecules from, e.g., the 3′-untranslated region (3′ UTR). Constructs can include but are not limited to the 5′ untranslated regions (5′ UTR) of an mRNA nucleic acid molecule, which can play an important role in translation initiation and can also be a genetic component in an expression construct. These additional upstream and downstream regulatory nucleic acid molecules may be derived from a source that is native or heterologous with respect to the other elements present on the promoter construct.

“Expression vector”, “vector”, “expression construct”, “vector construct”, “plasmid”, or “recombinant DNA construct” is generally understood to refer to a nucleic acid that has been generated via human intervention, including by recombinant means or direct chemical synthesis, with a series of specified nucleic acid elements that permit transcription or translation of a particular nucleic acid in, for example, a host cell. The expression vector can be part of a plasmid, virus, or nucleic acid fragment. Typically, the expression vector can include a nucleic acid to be transcribed operably linked to a promoter.

The term “genotype” means the specific allelic makeup of an organism.

“Highly stringent hybridization conditions” are defined as hybridization at 65° C. in a 6×SSC buffer (i.e., 0.9 M sodium chloride and 0.09 M sodium citrate). Given these conditions, a determination can be made as to whether a given set of sequences will hybridize by calculating the melting temperature (Tₘ) of a DNA duplex between the two sequences. If a particular duplex has a melting temperature lower than 65° C. in the salt conditions of a 6×SSC, then the two sequences will not hybridize. On the other hand, if the melting temperature is above 65° C. in the same salt conditions, then the sequences will hybridize. In general, the melting temperature for any hybridized DNA:DNA sequence can be determined using the following formula: Tₘ=81.5° C.+16.6(log₁₀[Na^{+]})+0.41(fraction G/C content)−0.63(% formamide)−(600/1). Furthermore, the Tₘ of a DNA:DNA hybrid is decreased by 1-1.5° C. for every 1% decrease in nucleotide identity [see Sambrook and Russel, 2006].

The term “introgressed,” when used in reference to a genetic locus, refers to a genetic locus that has been introduced into a new genetic background. Introgression of a genetic locus can thus be achieved through plant breeding methods and/or by molecular genetic methods. Such molecular genetic methods include, but are not limited to, various plant transformation techniques and/or methods that provide for homologous recombination, non-homologous recombination, site-specific recombination, and/or genomic modifications that provide for locus substitution or locus conversion.

The term “linked,” when used in the context of nucleic acid markers and/or genomic regions, means that the markers and/or genomic regions are located on the same linkage group or chromosome.

A “marker” means a detectable characteristic that can be used to discriminate between organisms. Examples of such characteristics include, but are not limited to, genetic markers, biochemical markers, metabolites, morphological characteristics, and agronomic characteristics.

A “marker gene” refers to any transcribable nucleic acid molecule whose expression can be screened for or scored in some way.

Certain genetic markers useful in the present disclosure include “dominant” or “codominant” markers. “Codominant” markers reveal the presence of two or more alleles (two per diploid individual). “Dominant” markers reveal the presence of only a single allele. The presence of the dominant marker phenotype (e.g., a band of DNA) is an indication that one allele is present in either the homozygous or heterozygous condition. The absence of the dominant marker phenotype (e.g., absence of a DNA band) is merely evidence that “some other” undefined allele is present. In the case of populations where individuals are predominantly homozygous and loci are predominantly dimorphic, dominant and codominant markers can be equally valuable. As populations become more heterozygous and multiallelic, codominant markers often become more informative of the genotype than dominant markers.

“Operably-linked” or “functionally linked” refers preferably to the association of nucleic acid sequences on a single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation. The two nucleic acid molecules may be part of a single contiguous nucleic acid molecule and may be adjacent. For example, a promoter is operably linked to a gene of interest if the promoter regulates or mediates transcription of the gene of interest in a cell.

The term “phenotype” means the detectable characteristics of a cell or organism that can be influenced by gene expression.

The term “population” means a genetically heterogenous collection of organisms that share a common parental derivation.

A “promoter” is generally understood as a nucleic acid control sequence that directs transcription of a nucleic acid. An inducible promoter is generally understood as a promoter that mediates transcription of an operably linked gene in response to a particular stimulus. A promoter can include necessary nucleic acid sequences near the transcription start site, such as, in the case of a polymerase II type promoter, a TATA element. A promoter can optionally include distal enhancer or repressor elements, which can be located as much as several thousand base pairs from the start site of transcription.

A “quantitative trait locus (QTL)” is a chromosomal location that encodes for alleles that affect the expressivity of a phenotype.

A “transcribable nucleic acid molecule” as used herein refers to any nucleic acid molecule capable of being transcribed into a RNA molecule. Methods are known for introducing constructs into a cell in such a manner that the transcribable nucleic acid molecule is transcribed into a functional mRNA molecule that is translated and therefore expressed as a protein product. Constructs may also be constructed to be capable of expressing antisense RNA molecules, in order to inhibit translation of a specific RNA molecule of interest. For the practice of the present disclosure, conventional compositions and methods for preparing and using constructs and host cells are well known to one skilled in the art [Sambrook and Russel, 2006; Ausubel et al.; Sambrook and Russel, 2001; Elhai and Wolk].

The “transcription start site” or “initiation site” is the position surrounding a nucleotide that is part of the transcribed sequence, which is also defined as position+1. With respect to this site all other sequences of the gene and its controlling regions can be numbered. Downstream sequences (i.e., further protein encoding sequences in the 3′ direction) can be denominated positive, while upstream sequences (mostly of the controlling regions in the 5′ direction) can be denominated as negative.

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”.

“Transformed,” “transgenic,” and “recombinant” refer to a host cell or organism such as a plant into which a heterologous nucleic acid molecule has been introduced. The nucleic acid molecule can be stably integrated into the genome as generally known in the art. Known methods of PCR include, but are not limited to, methods using paired primers, nested primers, single specific primers, degenerate primers, gene-specific primers, vector-specific primers, partially mismatched primers, and the like. The term “untransformed” refers to normal cells that have not been through the transformation process.

“Wild-type” refers to a virus or organism, or any of their components, found in nature without any known mutation.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein.

Nucleotide and/or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2 or Megalign (DNASTAR) software is used to align sequences. Those skilled in the art can determine appropriate parameters for measuring alignment, including any algorithms needed to achieve maximal alignment over the full-length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as: percent sequence identity=X/Y100, where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A.

In some embodiments, the terms “a” and “an” and “the” and similar references used in the context of describing a particular embodiment (especially in the context of certain of the following claims) can be construed to cover both the singular and the plural, unless specifically noted otherwise. When used in conjunction with the word “comprising” or other open language in the claims, the words “a” and “an” denote “one or more,” unless specifically noted.

In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present disclosure.

Having described the present disclosure in detail, it will be apparent that all of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and this can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1. Plasmid Construction

All PCR reactions were done using Q5@ Hot Start High-Fidelity 2× Master Mix (New England Biolabs) using the primers synthesized by Integrated DNA technologies (Primers used for constructions are given in TABLE 1). Hifi DNA assembly performed with NEBuilder® HiFi DNA Assembly Master Mix, Q5 mutagenesis using NEB Q5® Site-Directed Mutagenesis Kit and Infusion cloning using Takara In-Fusion® HD EcoDry Cloning kit following manufacturer's instructions. All the constructed plasmids were transformed into NEB 5-alpha F′I^{q} E. coli competent cells following manufacturer's instructions. Transformants were selected using LB agar plates containing 50 μg/mL kanamycin as the antibiotic marker and the plates were incubated at 37° C. overnight. Colony PCR was done to confirm the correct plasmid constructs and sequence confirmation is done by MCLAB DNA Sequencing.

TABLE 1
Primers used for plasmid construction in the experiments.

| Primer | | Plasmid | Target region used | Construction |
|---|---|---|---|---|
| ID | Primer sequence | construct | for amplification | method |
| oLJ189 | ggaattgtgagcggataacaatttc | pBLT-2 | g-Block, containing | NEB-Hifi DNA |
| | acactTCATCAAGTCAA | PETase | the Codon optimize | Assembly were |
| | AACACTATATAGGAA | MHETase | (P. putida), PETase | performed to |
| | CGAAACCATGAACTT | | and MHETase | construct the |
| | CCC (SEQ ID NO: 15) | | | plasmid, briefly, |
| | | | | the amplified |
| | | | | DNA fragment |
| | | | | with |
| oLJ190 | cgctggagtctgaggctcgtcctga | | | oLJ189/oLJ190 |
| | atgatCGGAGGCGCGGC | | | was added to |
| | GCAGGC (SEQ ID NO: 16) | | | the vector |
| | | | | (pBLT-2) |
| | | | | digested with |
| | | | | XbaI and EcoRV. |
| oLJLJ102 | TAACAAGGATTACAT | pBLT2- | Deletion of PETase | NEB-Q5 |
| | ATAAGG (SEQ ID NO: 17) | MHETase | from pBLT2- | mutagenesis |
| OLJLJ103 | AAGTGTGAAATTGTT | | PETase-MHETase | |
| | ATCCG (SEQ ID NO: 18) | | | |
| oLJLJ104 | ATCATTCAGGACGAG | pBLT2- | Deletion of | NEB-Q5 |
| | CCTC (SEQ ID NO: 19) | PETase | MHETase from | mutagenesis |
| oLJLJ105 | TTAGCTGCAGTTCGC | | pBLT2-PETase- | |
| | AGT (SEQ ID NO: 20) | | MHETase | |
| oLJLJ120 | CAAGGATTACATATA | pBLT2- | Amplification | Takara-Infusion |
| | AGGGTATATAAATGC | LCC- | pBLT-2 PETase- | cloning |
| | AGACCAC (SEQ ID NO: 21) | MHETase | MHETase to swap | |
| oLJLJ121 | AATCATACCTGACCT | | PETase with LCC | |
| | CCATAGCAGAAAG | | | |
| | (SEQ ID NO: 22) | | | |
| OLJLJ122 | AGGTCAGGTATGATT | pBLT2- | Amplification of | |
| | ACACTCTAGAGAGCT | LCC- | LCC fragment to | |
| | GTTGACAATTAATCA | MHETase | swap PETase in | |
| | T (SEQ ID NO: 23) | | pBLT2-PETase- | |
| OLJLJ123 | TATATGTAATCCTTGT | | MHETase | |
| | CACTGGCAGTGACGA | | | |
| | TTGTTGG (SEQ ID NO: 24) | | | |
| OLJLJ131 | ATGAGCAAGGGCGAG | pBLT2- | Amplification of | NEB-Hifi DNA |
| | GAG (SEQ ID NO: 25) | LCC-GFP | pBLT2-GFP vector | Assembly |
| oLJLJ132 | TTGAGGGGGATCCGC | | | |
| | CCT (SEQ ID NO: 26) | | | |
| OLJLJ133 | TGAGGGCGGATCCCC | pBLT2- | Amplification of | |
| | CTCAACGGATCCCCC | LCC-GFP | LCC fragment from | |
| | TCAAGTCAAAAG | | pBLT2-LCC- | |
| | (SEQ ID NO: 27) | | MHETase to tag | |
| oLJLJ134 | AGCTCCTCGCCCTTGC | | GFP with | |
| | TCATAGAACCTCCTCC | | GlyGlyGlySer | |
| | ACCTCCCTGGCAGTG | | linker | |
| | ACGATTGTTG (SEQ ID NO: 28) | | | |
| oLJLJ183 | ACGAGCCTCAGACTC | pBLT2- | Amplification of | NEB-Hifi DNA |
| | CAGCGGGAGGTGGAG | MHET- | linearized pBLT2- | Assembly |
| | GAGGTTCTATGAG | GFP | LCC-GFP vector | |
| | (SEQ ID NO: 29) | | | |
| oLJLJ184 | GTGCTGCCGCCGCCT | | | |
| | GCGCAAGCGGTGGCC | | | |
| | GCGGC (SEQ ID NO: 30) | | | |
| OLJLJ185 | TCAGCGCCGCGGCCA | pBLT2- | Amplification of | |
| | CCGCTTGCGCAGGCG | MHET- | MHETase from | |
| | GCGGC (SEQ ID NO: 31) | GFP | pBLT2-PETase- | |
| OLJLJ186 | ATAGAACCTCCTCCA | | MHETase without | |
| | CCTCCCGCTGGAGTCT | | secretion signal and | |
| | GAGGCTCG (SEQ ID | | stop codon | |
| | NO: 32) | | | |
| oLJLJ187 | TAGGATATCATTCAG | pBLT2- | Amplification of | NEB-Hifi DNA |
| | GACGAGCCTCAGA | fused | linearized pBLT2- | Assembly |
| | (SEQ ID NO: 33) | MHET- | MHET-GFP to | |
| oLJLJ188 | AGAACCTCCTCCACC | LCC | assemble LCC in C | |
| | TCCC (SEQ ID NO: 34) | | terminus-without | |
| | | | secretion signal | |
| OLJLJ189 | GGTGGAGGAGGTTCT | pBLT2- | Amplification of | |
| | ATGGACGGTGTACTG | fused | LCC from pBLT2- | |
| | TGGC (SEQ ID NO: 35) | MHET- | LCC-GFP without | |
| oLJLJ190 | CTGAATGATATCCTA | LCC | secretion signal and | |
| | CTGGCAGTGACGATT | | stop codon | |
| | GTTGGT (SEQ ID NO: 36) | | | |
| oLJLJ250 | GGAGGTGGAGGAGGT | pBLT2- | Amplification of | Takara-Infusion |
| | TCTATGG (SEQ ID NO: 37) | fused | pBLT2-LCC with | cloning |
| oLJLJ251 | GGTTTCGTTCCTATAT | MHET- | linker from pBLT2- | |
| | AGTGTTTTGACTTGAT | LCC | LCC-MHET | |
| | (SEQ ID NO: 38) | | | |
| oLJLJ252 | TATAGGAACGAAACC | pBLT2- | Amplification of | |
| | ATGCAGACCACCGTC | fused | MHETase with | |
| | ACC (SEQ ID NO: 39) | MHET- | secretion signal | |
| OLJLJ253 | ACCTCCTCCACCTCCC | LCC | from pBLT2- | |
| | GGAGGCGCGGCGCA | | PETase-MHETase | |
| | (SEQ ID NO: 40) | | | |
| OLJLJ243 | AGGAGGTGGAGGTGG | pBLT2- | Addition of extra | NEB-Q5 |
| | AGGAGGTTCTATGGA | fused | GlyGlyGlyGly | mutagenesis |
| | CGGTGTACT (SEQ ID | MHET- | linker to pBLT2- | |
| | NO: 41) | LCC- | fused MHET-LCC | |
| oLJLJ243 | CCACCTCCCGGAGGC | linker | | |
| A | GCGGCGCAGGC (SEQ | | | |
| | ID NO: 49) | | | |

### Example 2. Analysis of BHET Hydrolysis Products

Shake flask experiments were performed using M9 minimal medium (Fisher Scientific) containing 33.9 g/L disodium phosphate (anhydrous), 15.0 g/L monopotassium phosphate, 2.5 g/L sodium chloride, 5.0 g/L ammonium chloride, 4 mM magnesium sulphate, 36 μM ferrous sulphate, 200 μM calcium chloride supplemented with 20 mM glucose (Fisher Scientific) and 5 mM bis (2-hydroxyethyl) terephthalate (BHET) (Fisher Scientific). Overnight cultures of strains in LB medium supplemented with the antibiotics were harvested and washed with M9 medium. A fresh culture of 25 mL in 250 mL flasks were prepared with a starting OD₆₀₀ value of 1.0 and shake flasks were incubated at 30° C. shaking at 225 rpm. All the shake flask experiments were performed in 3 replicates. The concentrations of BHET and terephthalic acid (TPA) filtered standard solutions were measured using high performance liquid chromatography (HPLC) using Agilent 1100 system using Eclipse Plus C18 column (4.6×100 nm, 3.5 μm) (Agilent, USA). Acetonitrile (C) and 0.5% formic acid (D) were used as the mobile phase with a flow rate of 0.5 ml/min and maximum pressure of 4000.00 psi and the method parameters are as given in TABLE 2. The compounds were detected using diode array detector (DAD) and fluorescence detectors (FLD). All instrument control, data analysis and data processing were performed with Agilent OpenLAB control Panel software. Compounds were identified based on the retention times compared with that of the standards and the concentrations were calculated based on calibration curve generated for each sample by the software. HPLC analysis was done at time intervals 0, 6, 12, 24, 36, and 48 hours. Growth of the cultures was determined by measuring OD₆₀ value at each time point by using spectrophotometer (GENESYS 30, Thermo Scientific, USA).

TABLE 2
HPLC solvent gradient for 20 min run.

| Time (min) | C (%) | D (%) |
|---|---|---|
| 0.00 | 5.0 | 95.0 |
| 1.00 | 5.0 | 95.0 |
| 19.00 | 30.0 | 70.0 |
| 19.10 | 5.0 | 95.0 |

### Example 3. CRISPR-Cas9 Based-Gene Deletion in Erwinia aphidicola LJJL-01

Erwinia aphidicola LJJL01 gene deletion was done using CRISPR-Cas9 genome editing system. All electrocompetent competent cells used in this experiment were prepared using 0.3M sucrose. Overnight cultures of 5 mL LB with the antibiotics were used to make fresh LB cultures in the following day with OD₆₀₀ value of 0.1. Strains containing λ-Red plasmid (modified by our lab) and pX2-Cas9 plasmids (Addgene: Cat #85811) were induced with 10 mM arabinose and the culture was allowed to grow until it reaches an OD₆₀₀ value of 0.4. Competent cells were prepared as described by Franden et al., 2018. 5 μL of plasmids were transformed into 50 μL of electrocompetent cells in chilled electroporation cuvettes and electroporated at 1600V using Eppendorf Electroporator 2510 Five hundred microliters of SOC medium was used for recovery at 30° C. shaking at 225 rpm for 3 hours Transformation tubes were centrifuged at 14000 rpm for 1 minute and the cell pellet was dissolved in 1 mL LB broth with the appropriate antibiotics. 200 μL of the mixture is added to LB agar selection plates with the appropriate antibiotic and incubated at 30° C. overnight.

### Example 4. Deletion of Muconate Cycloisomerase Gene in E. aphidicola LJJL01(2752379084)

The above-mentioned CRISPR-Cas9 based method was adopted to delete the gene. The gRNA (with tetracycline resistance) carrying the spacer sequence of “TCTGGCGCAGTTGATATGTA” was constructed using Q5 mutagenesis, the SS9 gRNA plasmid was used as the template (Addgene Cat #71656). The sequenced verified gRNA and the repair DNA (attached the FASTA file) to delete the gene were used for the construction. The gene knockout colonies were identified on a 10 mg/mL tetracycline-containing LB plate. The deleted colony were verified with the diagnostic colony PCR with the primers oLJLJ038:

(SEQ ID NO: 47)
ACGAATTCGAGCTCGGTACCCGGGGATCCTATATGTGCCGGACGCCG,
oLJLJ041:
(SEQ ID NO: 48)
CGGCCAGTGCCAAGCTTGCATGCCTGCAGGTTGGCAGGCCGATGCCAAG.

The plasmids used for the CRISPR-Cas9 genome editing were cured by passing the strain on LB medium without antibiotics, and the glycerol stock of clean (without plasmids), E. aphidicola LJJL01 lacking muconate cycloisomerase was prepare and stored in −80° C. freezer.

The gRNA plasmid sequence is shown as SEQ ID NO: 1 in the Sequence Listing section below. The repair DNA sequence is shown as SEQ ID NO: 2 in the Sequence Listing section below.

Deletion of pcaIJ gene in P. putida KT2440: The gene was deleted using pK18mobsacB-based plasmid via SacB-based gene deletion method [Jha et al., 2018].

### Example 5. OHD Process of Waste Tea, Coffee, Corn Stover, and PET

Two hundred grams of commercially available green and black tea leaves or coffee purchased from the store (IG, Carbondale, IL, USA). Then, leaves were rinsed thoroughly with water, and then 1.0 Liter of boiling water was poured over 5 grams of dry leaves or coffee until water ran clear, the obtained solid waste Tea and Coffee were then subjected to grinding. 2 Liters of slurry was prepared by grinding the leaves in water at 1.0% dry solids. For the Corn Stover, or Corn Stover and plastic mix condition, the Corn Stover obtained from field (Carbondale, IL) were subjected to grinding as the method mentioned above. The Polyethylene terephthalate powder (crystallinity >45%) obtain from GoodFellow, USA were mixed with 95% (w/w) Corn Stover to obtain Corn Stover-plastic OHD mix. An OHD experiment was performed at 260° C., 2000 psi, with an Oxygen:Carbon mass ratio of 0.3:1.0, and residence time of 22 seconds in the reactor. 3 replicates were performed, collecting ˜900 mL each, and the product was combined after filtering to 0.8 micron using Millipore ATTP poly filters. All the samples were produced under the same set of conditions, no mass balance closure was performed. Conversion was roughly 80-90%, unconverted solids were filtered off. No char or solid carbon was produced. For the microbial testing, pH of the OHD was adjusted to 7.0 by adding NaOH, and the samples were filtered with 0.2 micron filters to sterilize the medium.

### Example 6. Identification and Quantification of the OHD Products

Identification and quantification of individual compounds contained in the OHD were undertaken based on the suite of methods developed by Dr. Anderson's laboratory, and the analytical methods adopted in thermochemical wastewater characterization to achieved 100% mass closure. Briefly, organic products of corn stover OHD were extracted from the raw liquor with ethyl acetate and analyzed by with in-situ derivatization using Tetramethylammonium hydroxide for methylation of acidic oxygen functional groups. (Sanders, 2017) GC-MS analyses of OHD products were performed on an Agilent Technologies 7890A GC system equipped with a 5975C inert XL MS detector and a 7683B series injector. The GC system contains a 60 m Zebron ZB-1701 column, 0.25 mm internal diameter, and 0.25 μm film thickness. Analyses were performed using He carrier gas flow controlled to 1 ml/min in constant flow mode and the GC oven was temperature programmed as follows: initial temperature of 40° C. held for 4 min, increased at 4° C./min to 280° C., held for 15 min. The MS and transfer line will be programmed to 150° C. and 250° C., respectively. The MS scanned a range of m/z 10 to 400 to record full spectra. Data analysis were performed using Agilent software. Identification was based on a comparison of spectra with the Wiley and National Institute of Standards and Technology (NIST) mass spectral libraries, literature data, comparison with standards and interpretation. The method was successfully used to identify and quantify the ˜35 organic compounds from cane bagasse OHD. All analyses were performed in triplicate experiments, and all quantitative standard curves were maintained with an R² value of ≥0.995 with five or more points of reference concentrations. Individual analytical grade standards were used to construct the calibration curve, and select internal standards will be added to adjust for chromatographic and detector response shift.

### Example 7. Monitoring Growth and Conversion of OHD Substrates by Microbes

To assess the growth both 96 well-plate and 50 mL shake flask experiments were performed using 50 mL baffled flasks containing 50 mL modified M9 media supplemented with 1% (v/v) FPF (pH 7) and inoculated to OD₆₀₀ 0.2 with cells prepared as above but resuspended in M9 medium containing 1% (v/v) FPF. Cultures were incubated shaking at 225 rpm, 30° C. 2 mL samples were collected periodically and subjected to HPLC analysis, and OD₆₀₀ growth measurement using a Beckman DU640 spectrophotometer (Beckman Coulter, Brea CA) or MPlex plate reader (TECAN, USA). The above-mentioned the HPLC method (Section 2) was adopted to detect the compounds in OHD during the microbial conversion. We monitored the β-ketoadipate production with the biosensor previously developed by Jha and coworkers [Jha et al., 2018].

### Example 8. Monitoring Growth on Bacterial OHD

To evaluate the potential of using Bacterial-OHD as a growth media, we grow the P. putida KT2440 and E. aphidicola LJJL01 bacterial cells on 1 L LB medium, 30°, 225 rpm, and overnight. Cells were harvested by centrifugation (4000 rpm at 10 min), and subjected to the OHD process as described in Example 5. Next, we used different concentrations of Bacterial OHD and tested the growth off P. putida KT2440 and E. aphidicola LJJL01 with/without supplement M9 medium. Two hundred milliliters of the medium in a 96-well plate were inoculated with the strain (OD600=0.1), and incubated at maximum shaking, 30° C., using a Mplex plate reader (TECAN, USA) to monitor the growth.

### Example 9. Engineered E. aphidicola LJJL01 to Produce Muconate from OHD

Biological production of cis, cis-muconate was demonstrated with purified sugars or lignin-derived aromatics via engineered microbes. Fundamentally, the CatA/B deletion enables the accumulation of muconate [Johnson et al., 2019; Bentley et al., 2020]. Also, microbial systems have been engineered to produce muconate by introducing heterologous genes [Leavitt et al., 2017].

This is the first report of the utilization of E. aphidicola LJJL01 to produce muconate from OHD substrates (unpurified heterogeneous compounds). The potent genes, 2752379084 (sequence given, only 56 similarities to CatA, muconate cycloisomerase of P. putida [see FASTA the sequence]). We knocked out and developed the strain to produce cis,cis-muconate from aromatics molecules (FIG. 1). The production of cis-cis muconate from Catechol was demonstrated. The potent pathways of production of muconate from OHD-plastic & biomass described. (FIG. 1).

Given that E. aphidicola LJJL01 exhibits high tolerance to most of the toxic chemicals, the engineered organism will serve as one of the fundamental chassis or developing high-value bioproducts such as muconate from OHD substrates or aromatics.

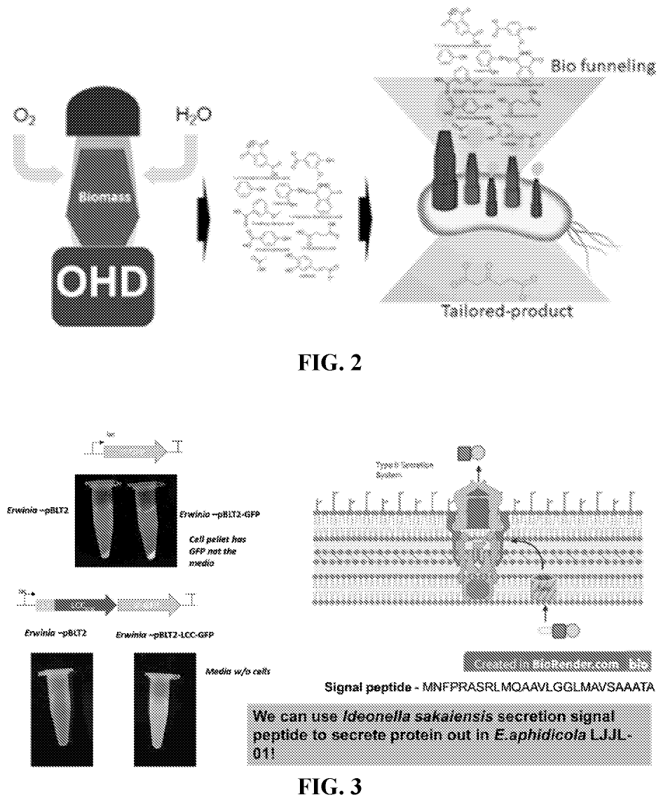

FIG. 2 shows the developed integrated process of OHD and engineered monoculture to funnel heterogeneous OHD products (aromatics, sugars, acids) to a single product (i.e. beta-ketoadipate). (˜90% yield from OHD aromatics).

### Example 10. Engineered Microbes to Selectively Degrade Plastic

Microbes have been engineered to degrade plastic into original monomers. However, substrates, intermediates, and product toxicity hamper the efforts. In addition, the limitation of secretion of the plastic degradation enzymes and selectivity of degradation are key hurdles to developing efficient cell factories [Jayakody & Dissanayake, 2021].

The French company Carbois, created the efficient enzyme, Leaf-branch compost cutinase (LCC), to degrade plastic at high temperature (80° C.) [Tournier et al., 2020].

Previous teams have developed P. putida KT2440 to degrade the PET selectively by secreting the PETase and MHETase using I. sakainesis secretion peptides.

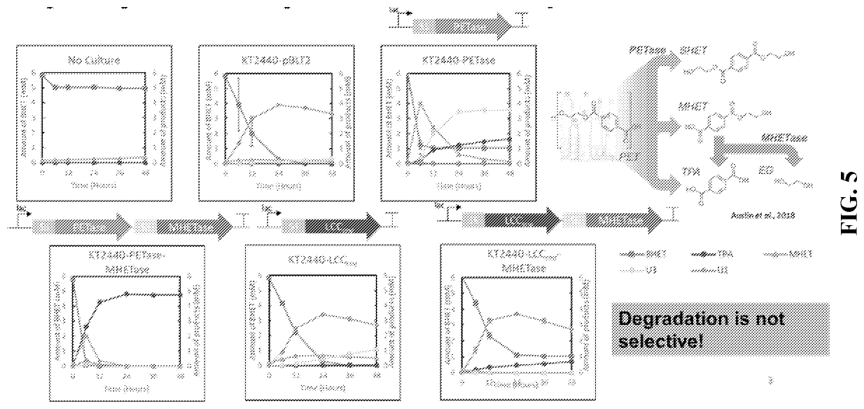

We demonstrated the E. aphidicola LJJL01 is a better platform organism relative to P. putida for selectively degrade PET. For instance, BHET can be 100% selectively degraded by expressing the codon-optimized engineered LCC enzyme (see the sequences) and the MHETase enzyme at 30° C. (FIGS. 3-6).

For the first time, we developed E. aphidicola LJJL01 to secrete the plastic degradation enzymes using secretion signal peptides originated from I. sakaiensis. (FIGS. 3 and 7) We simultaneously secret the enzymes efficiently, and enable efficient degradation (FIG. 4).

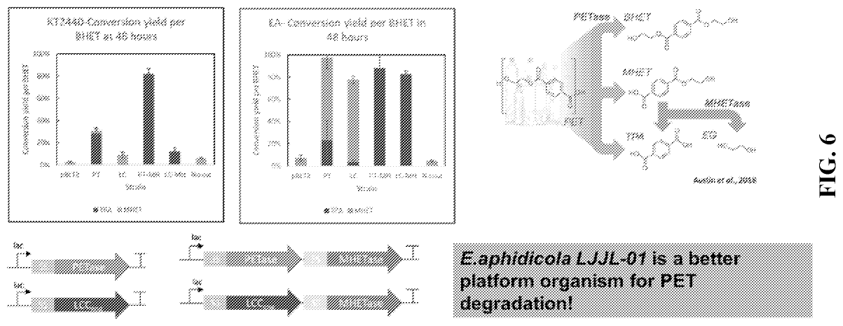

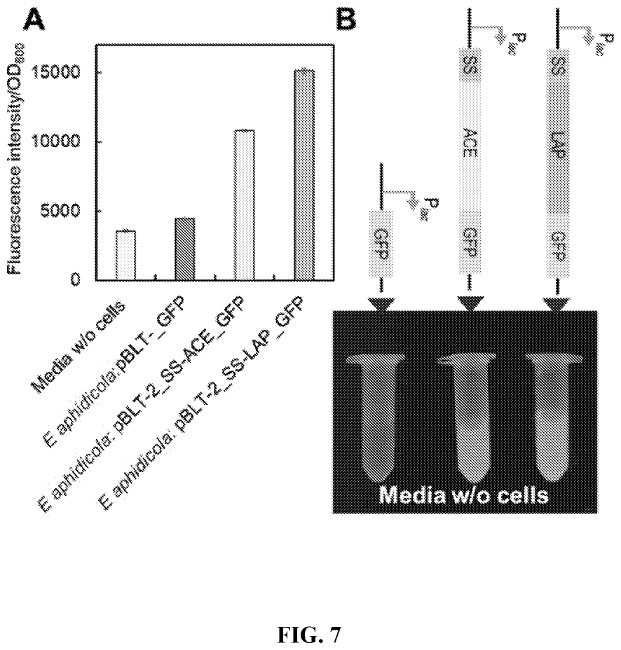

We discovered the synergistic effect of LCC and MHETase to degrade BHET. (FIG. 6).

We developed the whole-cell biocatalyst (E. aphidicola LJJL01) to degrade PET and other plastics efficiently.

In addition to PET degradation enzymes, we successfully express and secreted fungal-originated polyurethane degradation enzymes in E. aphidicola LJJL01. (FIG. 7).

The plasmid map of the LCC and MHETase is shown in FIG. 8, and the corresponding pBLT-2:Ptac-LCC_MHETase plasmid gene sequence is shown as SEQ ID NO. 7.

The new synthetic LCC expression cassette sequence is shown as SEQ ID NO. 8.

A plasmid map of the expression of fungal PU-degradation genes in E. aphidicola LJJL01 design is shown in FIG. 9, and the corresponding ACE-540972 plasmid gene sequence is shown as SEQ ID NO. 9.

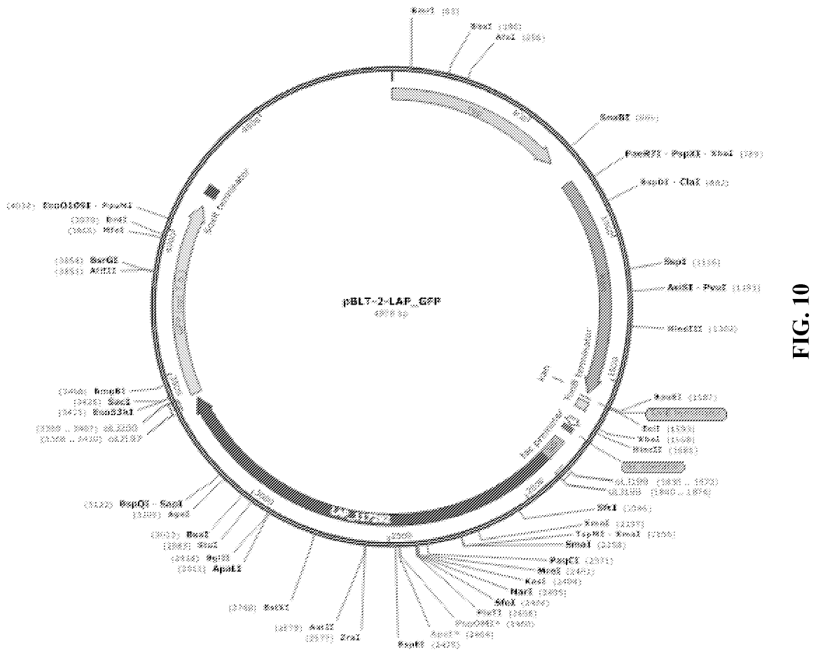

An additional plasmid map of the expression of fungal PU-degradation genes in E. aphidicola LJJL01 design is shown in FIG. 10, and the corresponding LAP_117292 plasmid gene sequence is shown as SEQ ID NO. 10.

FIG. 11 shows the developed in situ degradation of PET without chemical catalyst of external supplementation of chemical (diol/acids), and the integrated OHD and engineered microbes to recover the original plastic monomer (i.e., TPA).

### Example 11. Hybrid OHD-Microbe Process to Recycle or Upcycle PET

Given that high-crystallinity of PET, it is challenging to implement fully biological process to recover the monomer directly from commercial PET. Pyrolysis (high-temperature, costly process) and chemical deconstruction (expensive catalysts/harsh solvents) process have been developed to recover the monomers [Bhaskar et al., 2004; Shojaei et al., 2020; Walker et al., 2020]. Also, researchers developed the microbes to utilize those monomers and enable the plastic upcycling [Tiso et al., 2020; Kenny et al., 2008].

We developed the OHD process to solubilize high-crystalline PET (>45%) by mixing it with biomass. Thus, the novel process can apply to real-world waste (mix waste streams).

We demonstrated the both P. putida and E. aphidicola LJJL01 can grow on PET-Corn Stover OHD substrate. Also, if we use the engineered E. aphidicola LJJL01 or P. putida KT2440 harboring the PET degradation enzymes, they can convert BHET and MHET in the OHD to TPA selectively (FIGS. 12 and 13). (Enable OHD-microbes hybrid recirculation strategy of PET). The recovered TPA can be upcycled into various chemicals; indeed muconate and PDC (FIG. 14). Several other upcycling routes can be developed in E. aphidicola LJJL01.

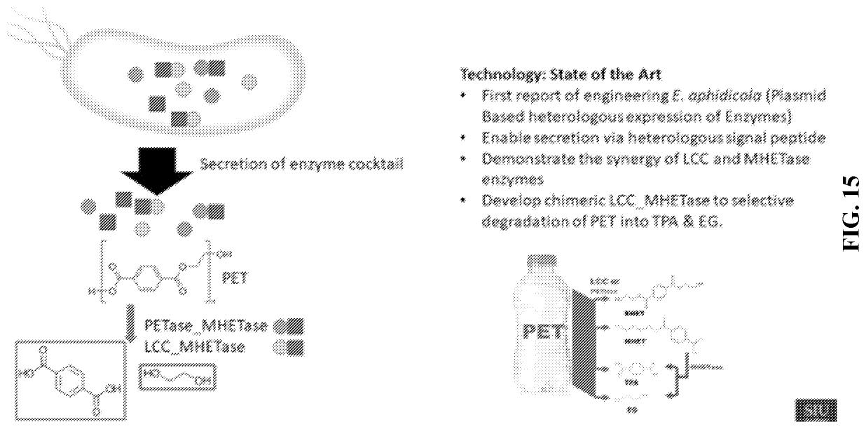

FIG. 15 shows the selectively designed microbe for depolymerization of PET.

### Example 12. Strain Construction

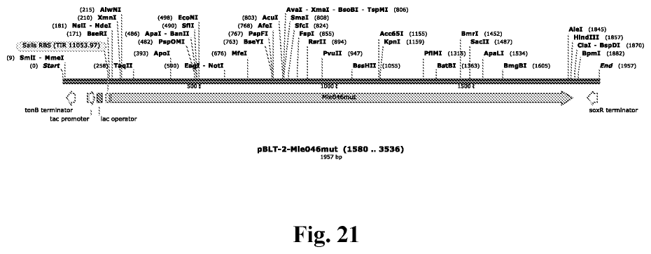

For the in vivo experiments, pBLT2-LCC-Mle046 plasmid (FIG. 20, SEQ ID NO: 11) was constructed using HiFi DNA assembly method. This plasmid was then transformed into E. coli DH5α-Iq strain using the heat shock method. Colonies were selected using LB agar plates supplemented with 50 μg/mL kanamycin. The colonies were screened by a colony PCR and visualized using gel electrophoresis. The plasmids were then extracted, and sequence verified. Q5 mutagenesis was performed to create the pBLT2-LCC-Mle046(mutant) (FIG. 21, SEQ ID NO: 12 and SEQ ID NO: 13) with the glycine in position 131 swapped out for a serine using primers as shown in the table below. Transformants were selected using LB agar plates supplemented with 50 μg/mL kanamycin and sequence verified. Correct plasmids were transformed into E. aphidicola LJJL01 using electroporation and selection was done with LB-kanamycin plates.

TABLE 3
Oligo (overlap-black, blue-PCR specific)-5′-3′

| | | Template | | |
|---|---|---|---|---|
| Primer | | used for | Plasmid | Construction |
| ID | Primer sequence | PCR | constructed | method |
| LJ 565 | CCTGGAACGCagtTTCGCAGTGG | PLATE31 | Mle046mut- | Q5 |
| | (SEQ ID NO: 42) | | Fw | |
| LJ 566 | GCACTATCTTCCTGATTCAGG | pLATE31 | Mle046mut- | Q5 |
| | (SEQ ID NO: 43) | | Rw | |

### Example 13. Shake Flask Experiments

Shake flask experiments for assaying the BHET degradation were performed using 2×M9 minimal medium at pH 8, supplemented with 10 g/L glucose, salts, 1 mM bis (2-hydroxyethyl) terephthalate (BHET), 50 μg/mL kanamycin and 1 μM IPTG. Strains used for the study were E. aphidicola-pBLT2, E. aphidicola-pBLT2-LCC, E. aphidicola-pBLT2-LCC-Mle046 and E. aphidicola-pBLT2-LCC-Mle046(mutant). Overnight cultures of strains in LB medium supplemented with the antibiotics were harvested and washed with M9 medium. A fresh culture of 25 mL in 250 mL flasks were prepared with an initial OD₆₀₀ value of 1.0 and shake flasks were incubated at 30° C. shaking at 225 rpm. All the shake flask experiments were performed in 2 replicates. The conversions were monitored by means of a HPLC analysis using Shimadzu LC-2050C 3D coupled to a PDA detector. An isocratic elution program was used with 0.5% formaic acid in water and acetonitrile (80:20) as the mobile phases. The total run time was 10 minutes. Compounds were identified based on the retention times compared with that of the standards. Quantification was done based on the matrix matched calibration curves generated for each analyte using standard solutions prepared in 2×M9. Chromatograms were analyzed using LabSolutions DB software. HPLC analysis was done at time intervals 0, 24, 48 and 72 hours. Growth of the cultures were determined by TECAN Sunrise plate reader—Infinite M Flex.

Shake flask results are expressed as average values of the duplicates data with their standard errors of the means (SEM). Multiple comparisons were done using one-way analysis of variance (ANOVA) followed by Tukey's post hoc honest significance difference test. (https://astatsa.com/OneWay_Anova_with_TukeyHSD/).

The purified Mle046 mutant did not have a high concentration to register. Therefore no in vitro assays were completed, preventing us from getting biochemical properties of the Mle046 (mutant).

### Example 13. Results and Discussion In-Vivo Investigation of the Synergy of Mle046 and its Mutant with LCC on BHET Degradation

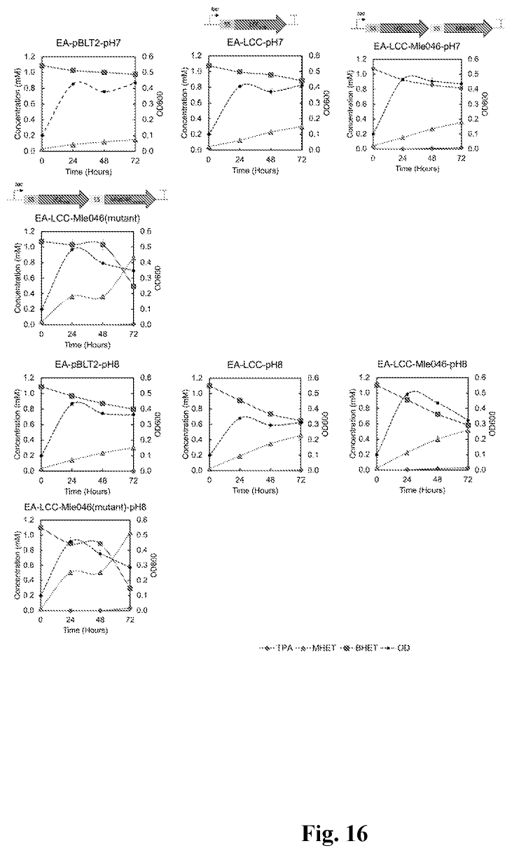

In FIG. 16, EA-LCC-Mle046 shows a synergistic effect against BHET compared to EA-LCC or other controls. Indeed, the TPA was detected only in the presence of Mle046, or its mutant. Notably, EA-LCC-Mle046 (mutant) yields 2×MHET (˜0.5 mM vs 0.25 mM after 72 h) from BHET relative to the EA-LCC-Mle046, but did not improve the TPA yield. The results suggest that the newly developed mutant has PETase activity. Given that both LCC and Mle046 activities are optimum at pH 8, the strains incubated at pH 8 exhibit higher conversion of BHET relative to pH 7.1 Of note, the host strain E. aphidicola LJJL01 can grow well in both pH 7 and pH 8, allowing this efficient conversion of BHET at pH 8. We demonstrated the synergistic activity of the dual enzyme system (PETase, and MHETase from I. sakaiensis) on selective plastic degradation using an in-vitro study.³ To the best of our knowledge, this is the first report demonstrating the synergistic activity of LCC and Mle046 on BHET degeneration by in-vivo study at ambient temperature. Of note, Mrigwani and coworkers demonstrate the synergy of LCC and thermostable Thermus thermophilus carboxylesterase at 60° C.⁴

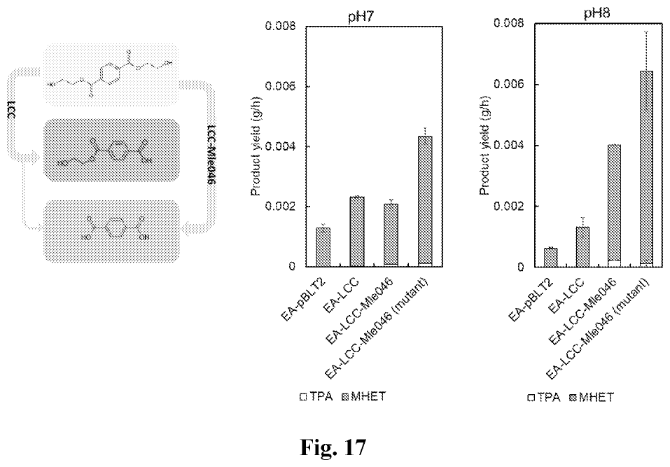

In FIG. 17, the samples at pH 7 versus the samples at pH 8 show that TPA conversion is increase by about 33% with the pH difference.

### Example 14. Investigation of the Synergy of Mle046 and its Mutant with LCC on MHET Degradation

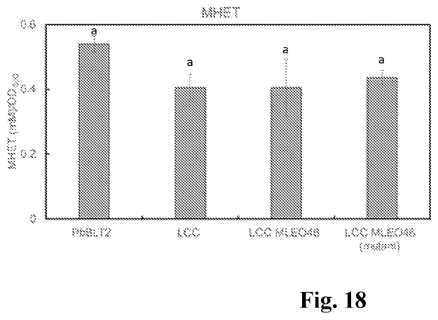

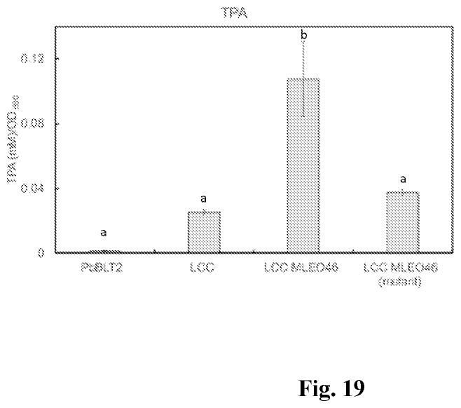

In FIG. 18, the efficiency of the enzymes in MHET degradation is given. There is no statistically significant difference between the four variants (0.05>p). In FIG. 19, the efficiency of the enzymes in TPA production are demonstrated, and here it is clear that EA-LCC-Mle046 (wild-type) has a much better conversion yield than the other three variants and shows statistical significance (0.05>p).

### Example 15. Discussion of Examples 12-14

We uncovered the Mle046 synergistic activity with LCC for selective degradation of PET and an engineering approach to modulate substrate specificity of Mle046 by mutating the catalytic domain (i.e., introducing PETase activity). Moving forward, more enzyme characterization through x-ray crystallography, in vitro assays to determine biochemical properties, and molecular dynamics studies will allow us to find better mutations to improve this enzyme and work towards a circular PET economy to serve our world better.

Sequence Listing

SEQ ID NO: 1
LENGTH: 2885
TYPE: RNA
ORGANISM: artificial
OTHER INFORMATION: gRNA plasmid sequence

| | |
|---|---|
| gaattctaaa gatctttgac agctagctca gtcctaggta taatactagt | 50 |
| tctggcgcag ttgatatgta gttttagagc tagaaatagc aagttaaaat | 100 |
| aaggctagtc cgttatcaac ttgaaaaagt ggcaccgagt cggtgctttt | 150 |
| tttgaagctt gggcccgaac aaaaactcat ctcagaagag gatctgaata | 200 |
| gcgccgtcga ccatcatcat catcatcatt gagtttaaac ggtctccagc | 250 |
| ttggctgttt tggcggatga gagaagattt tcagcctgat acagattaaa | 300 |
| tcagaacgca gaagcggtct gataaaacag aatttgcctg gcggcagtag | 350 |
| cgcggtggtc ccacctgacc ccatgccgaa ctcagaagtg aaacgccgta | 400 |
| gcgccgatgg tagtgtgggg tctccccatg cgagagtagg gaactgccag | 450 |
| gcatcaaata aaacgaaagg ctcagtcgaa agactgggcc tttcgtttta | 500 |
| tctgttgttt gtcggtgaac tggatcctta ctcgagtcta gactgcaggc | 550 |
| ttcctcgctc actgactcgc tgcgctcggt cgttcggctg cggcgagcgg | 600 |
| tatcagctca ctcaaaggcg gtaatacggt tatccacaga atcaggggat | 650 |
| aacgcaggaa agaacatgtg agcaaaaggc cagcaaaagg ccaggaaccg | 700 |
| taaaaaggcc gcgttgctgg cgtttttcca taggctccgc ccccctgacg | 750 |
| agcatcacaa aaatcgacgc tcaagtcaga ggtggcgaaa cccgacagga | 800 |
| ctataaagat accaggogtt tccccctgga agctccctcg tgcgctctcc | 850 |
| tgttccgacc ctgccgctta ccggatacct gtccgccttt ctcccttcgg | 900 |
| gaagcgtggc gctttctcat agctcacgct gtaggtatct cagttcggtg | 950 |
| taggtcgttc gctccaagct gggctgtgtg cacgaacccc ccgttcagcc | 1000 |
| cgaccgctgc gccttatccg gtaactatcg tcttgagtcc aacccggtaa | 1050 |
| gacacgactt atcgccactg gcagcagcca ctggtaacag gattagcaga | 1100 |
| gcgaggtatg taggcggtgc tacagagttc ttgaagtggt ggcctaacta | 1150 |
| cggctacact agaaggacag tatttggtat ctgcgctctg ctgaagccag | 1200 |
| ttaccttcgg aaaaagagtt ggtagctctt gatccggcaa acaaaccacc | 1250 |
| gctggtagcg gtggtttttt tgtttgcaag cagcagatta cgcgcagaaa | 1300 |
| aaaaggatct caagaagatc ctttgatctt ttctacgggg tctgacgctc | 1350 |
| agtggaacga aaactcacgt taagggattt tggtcatgag attatcaaaa | 1400 |
| aggatcttca cctagatcct tttaaattaa aaatgaagtt ttaaatcaat | 1450 |
| ctaaagtata tatgagtaaa cttggtctga cagttgacag cttatcatcg | 1500 |
| ataagcttta atgcggtagt ttatcacagt taaattgcta acgcagtcag | 1550 |
| gcaccgtgta tgaaatctaa caatgcgctc atcgtcatcc tcggcaccgt | 1600 |
| caccctggat gctgtaggca taggcttggt tatgccggta ctgccgggcc | 1650 |
| tcttgcggga tatcgtccat tccgacagca tcgccagtca ctatggcgtg | 1700 |
| ctgctagcgc tatatgcgtt gatgcaattt ctatgcgcac ccgttctcgg | 1750 |
| agcactgtcc gaccgctttg gccgccgccc agtcctgctc gcttcgctac | 1800 |
| ttggagccac tatcgactac gcgatcatgg cgaccacacc cgtcctgtgg | 1850 |
| atcctctacg ccggacgcat cgtggccggc atcaccggcg ccacaggtgc | 1900 |
| ggttgctggc gcctatatcg ccgacatcac cgatggggaa gatcgggctc | 1950 |
| gccacttcgg gctcatgagc gcttgtttcg gcgtgggtat ggtggcaggc | 2000 |
| cccgtggccg ggggactgtt gggcgccatc tccttgcatg caccattcct | 2050 |
| tgcggcggcg gtgctcaacg gcctcaacct actactgggc tgcttcctaa | 2100 |
| tgcaggagtc gcataaggga gagcgtcgac cgatgccctt gagagccttc | 2150 |
| aacccagtca gctccttccg gtgggcgcgg ggcatgacta tcgtcgccgc | 2200 |
| acttatgact gtcttcttta tcatgcaact cgtaggacag gtgccggcag | 2250 |
| cgctctgggt cattttcggc gaggaccgct ttcgctggag cgcgacgatg | 2300 |
| atcggcctgt cgcttgcggt attcggaatc ttgcacgccc tcgctcaagc | 2350 |
| cttcgtcact ggtcccgcca ccaaacgttt cggcgagaag caggccatta | 2400 |
| tcgccggcat ggcggccgac gcgctgggct acgtcttgct ggcgttcgcg | 2450 |
| acgcgaggct ggatggcctt ccccattatg attcttctcg cttccggcgg | 2500 |
| catcgggatg cccgcgttgc aggccatgct gtccaggcag gtagatgacg | 2550 |
| accatcaggg acagcttcaa ggatcgctcg cggctcttac cagcctaact | 2600 |
| tcgatcattg gaccgctgat cgtcacggcg atttatgccg cctcggcgag | 2650 |
| cacatggaac gggttggcat ggattgtagg cgccgcccta taccttgtct | 2700 |
| gcctccccgc gttgcgtcgc ggtgcatgga gccgggccac ctcgacctga | 2750 |
| cacatttccc cgaaaagtgc cacctgacgt ctaagaaacc attattatca | 2800 |
| tgacattaac ctataaaaat aggcgtatca cgaggcagaa tttcagataa | 2850 |
| aaaaaatcct tagctttcgc taaggatgat ttctg | 2885 |

SEQ ID NO: 2
LENGTH: 2068
TYPE: DNA
ORGANISM: artificial
OTHER INFORMATION: repair DNA sequence

| | |
|---|---|
| acgaattcga gctcggtacc cggggatcct atatgtgccg gacgccgacc | 50 |
| gtgccgcccc agctgattga gcagatgctg caccggcccg tcaggggggc | 100 |
| agccgtaccc tgacggcacg acgctgcgga actgatagcg cccctgttca | 150 |
| tcggtaatga tggtacgacg caggttaaac gcactctgac ttttatcgaa | 200 |
| gaacgagtaa ttaccgaggg tattggcgtg ccagacctca acgctggcgc | 250 |
| cgggaagcgg ttgaccctct tcgttccata ccgtgccctg catcaacagc | 300 |
| acttcgccct ggttatcttc agaaccatcg tccagccgcg cccaggcctg | 350 |
| acagactggc gccccggcca catagagagg accttcgatg gtgcgcggtg | 400 |
| tcccaccgtg cagccccgcc ctcgcttcag cctcatccgc ccggatatcc | 450 |
| ataaaacgct ccagtccggt tcccgcagcc agcagcccca tctcctgagc | 500 |
| gataccggta tcggccagat attccagccc tttccagatc tcagtttgct | 550 |
| gcaggcccag atcctcaatc gctttaaaca gatcgctcag gagacgaacg | 600 |
| gtgacctgct gaatacgtgg gttcaccgtg cctttcgcgg ttttgacgat | 650 |
| aaagcagtta accaggttgt cgatctcttt gctgttcata ttgacgctcc | 700 |
| atttcaggta cagctcagga tgagaaagta tttaacgatc gtccacacgc | 750 |
| accgccgaag ggtggcggca cagcggcact atctgcatcg tcataaaggg | 800 |
| ataaagcggc agctcactca gcagctggtg caactcctca acgctatcga | 850 |
| cgtcaaatac gctgacgttg gcgtaatgcc cggcaatgcg ccagatgtgt | 900 |
| cgccacttgc cgcgacgctg aagcgcctgc gagtaggctt tttcccgtgc | 950 |
| cttaagttcg ctggcgacat tttccgccat cgtctgcggc agattcacat | 1000 |
| ccatagttac gtgaaatagc atggtgaaat ttcctcctac gctatagaat | 1050 |
| cccgccgatc ggggatagcc aaatcctgct gaaaatgatt gacccgctgt | 1100 |
| atgagaatgc gcagatcgcg cctgttcgcc ccagctcaca taacttgcga | 1150 |
| taaaaacgtc ttttagtgcc aatgacactt ttaggttatt ttttcgccgg | 1200 |
| tgaagtcagc accctgatga ctcaaactca ggaggagcga gatgatcgac | 1250 |
| aaaagtctgt ttttggccga tgaagccgtg gctgacattc acgatggtgc | 1300 |
| cacgctgatg attggoggat ttggcccggc aggacagccc tatgcgctgc | 1350 |
| tggacgcact gattcggcgg cggccacaga acctcacgct ggtcagcaac | 1400 |
| aacgctggca acgctgattc gggtctggcg ctactgctca aagcggggtg | 1450 |
| cgtgcgcaag atgatctgtt cctttccgcg ccagagtgac tcatgggtat | 1500 |
| ttgacgacct ctaccgccgc ggagagattg agctggagct ggtgccgcag | 1550 |
| ggcaatcttg tggcgcgtat tcaggcaggt ggcagcgggc tgggggccat | 1600 |
| ttttacccca acgggtttcg gtactgaact ggctgcgggc aaagagacgc | 1650 |
| gcgaaattga ggggcggcag tacgtgctgg aactggcgct gaaggcagac | 1700 |
| tttgccctga tcaaagcaca gtgcggtgat cgctggggta acctggtcta | 1750 |
| tagcaaagcg gcacgcaact tcaacccgat tatggcgatg gctgcccgct | 1800 |
| gcaccatcgc tgaagtctcc cgccacgtcg cgctgggcaa gctggacccg | 1850 |
| gaggcggtgg tgactcccgg tatttttgtg cagcgcgtgg tctgttcagc | 1900 |
| caatctcagc cccgcaacga tcgcctcagg agcgtgacac aatgcaactc | 1950 |
| atccgcctca cgcataacca gctggcgcag cgcatcgccc gcgatatccc | 2000 |
| ggacggagcc tacgtcaatc ttggcatcgg cctgccaacc tgcaggcatg | 2050 |
| caagcttggc actggccg | 2068 |

SEQ ID NO: 3
LENGTH: 1110
TYPE: DNA
ORGANISM: bacteria
OTHER INFORMATION: E. aphidicola LJJL01muconate cycloisomerase
gene sequence (Gene ID: 2752379084)

| | |
|---|---|
| ttaacggcgg cgcagcgccg cgatcctgtc gcgatcgagg gcaatgccca | 50 |
| gcccgggccc ggtcgggacc tgcagcataa aatcacgata aactagcggt | 100 |
| tcactgagaa tatcttcggt cagcagcagc gggccaaaca gctcagtgcc | 150 |
| gaaactcaaa tcgttgaagg tggcgcagag atgcgccgtg gcggccgtgc | 200 |
| ctacggcgcc ttccagcatg gtgcccccgt aaagcgcgat atctgccagc | 250 |
| tgggcaatat cggcaacccg gcgcgcctgc gtcagcccgc cggactgggt | 300 |
| aatttttatc gagaacacgt cggcggcagc gtggcgggcc agctcgaaag | 350 |
| catcgcgcgg ccctttcagc gcttcgtccg cgatcactgg caggctgaaa | 400 |
| cggcgggtca gtcgcgccag tccggcacga ttttctgccg caatcggctg | 450 |
| ctcaacggca tcaatgccgc cggcttccag tgctgccatt ccgcgctcgg | 500 |
| cctggcgctc gctccaggcc tggttgacgt cgacgcgcac gctgacctca | 550 |
| tcccccaacg cctgcctgat ggccagcgcg tgcgccacgt ccgcatcaac | 600 |
| atcgcgcagg ccgattttga gtttaaaaat acgatgacgg cgcagggcta | 650 |
| acatctgccg ggcttcggca atatctttat cggtgctgcc gctggccaac | 700 |
| gtccaggcca ccggtagcgc atcgcgcacc cgtcctccca gcagttcact | 750 |
| gagcgcaata ttcagacgcc gtgcctgagc gtccagcaag gcggtttcaa | 800 |
| ttgcgcattt agcaaagcgg ttgccctgca ctgatttatt caggcgcgcc | 850 |
| atcagctggg cgatgcggcg cgcatcctga ccgatcagta acggcgtcat | 900 |
| ccagcggtca atattgactt tgacgctctc ggggctttcg tcgccgtagc | 950 |
| tcaggccacc aatggtggtg gcttctcccc agccctcaaa cccgtcctca | 1000 |
| cacaccagat ggaccagcac cagcgtttgc acctgaagcg tggccattgc | 1050 |
| caggcgatgt ggacggatag caggaacatc aataagcagc gtttcaatag | 1100 |
| ttttgatcat | 1110 |

SEQ ID NO: 4
LENGTH: 369
TYPE: protein
ORGANISM: bacteria
OTHER INFORMATION: E. aphidicola LJJL01Muconate cycloisomerase
protein sequence (Gene ID: 2752379084)

| | |
|---|---|
| MIKTIETLLI DVPAIRPHRL AMATLQVQTL VLVHLVCEDG | 40 |
| FEGWGEATTI GGLSYGDESP ESVKVNIDRW MTPLLIGQDA | 80 |
| RRIAQLMARL NKSVQGNRFA KCAIETALLD AQARRLNIAL | 120 |
| SELLGGRVRD ALPVAWTLAS GSTDKDIAEA RQMLALRRHR | 160 |
| IFKLKIGLRD VDADVAHALA IRQALGDEVS VRVDVNQAWS | 200 |
| ERQAERGMAA LEAGGIDAVE QPIAAENRAG LARLTRRFSL | 240 |
| PVIADEALKG PRDAFELARH AAADVFSIKI TQSGGLTQAR | 280 |
| RVADIAQLAD IALYGGTMLE GAVGTAATAH LCATFNDLSF | 320 |
| GTELFGPLLL TEDILSEPLV YRDFMLQVPT GPGLGIALDR | 360 |
| DRIAALRRR | 369 |

SEQ ID NO: 5
LENGTH: 320
TYPE: protein
ORGANISM: bacteria
OTHER INFORMATION: LCC protein sequence (signal peptide shown in bold)

| | |
|---|---|
| MNFPRASRLM QAAVLGGLMA VSAAATAMDG VLWRVRTAAL | 40 |
| MAALLALAAW ALVWASPSVE AQSNPYQRGP NPTRSALTAD | 80 |
| GPFSVATYTV SRLSVSGFGG GVIYYPTGTS LTFGGIAMSP | 120 |
| GYTADASSLA WLGRRLASHG FVVLVINTNS RFDGPDSRAS | 160 |
| QLSAALNYLR TSSPSAVRAR LDANRLAVAG HSMGGGGTLR | 200 |
| IAEQNPSLKA AVPLTPWHTD KTFNTSVPVL IVGAEADTVA | 240 |
| PVSQHAIPFY QNLPSTTPKV YVELCNASHI APNSNNAAIS | 280 |
| VYTISWMKLW VDNDTRYRQF LCNVNDPALC DFRTNNRHCQ | 320 |

SEQ ID NO: 6
LENGTH: 613
TYPE: DNA
ORGANISM: bacteria
OTHER INFORMATION: MHET protein sequence (signal peptide shown in bold)

| | |
|---|---|
| MQTTVTTMLL ASVALAACAG GGSTPLPLPQ QQPPQQEPPP | 40 |
| PPVPLASRAA CEALKDGNGD MVWPNAATVV EVAAWRDAAP | 80 |
| ATASAAALPE HCEVSGAIAK RTGIDGYPYE IKFRLRMPAE | 120 |
| WNGRFFMEGG SGTNGSLSAA TGSIGGGQIA SALSRNFATI | 160 |
| ATDGGHDNAV NDNPDALGTV AFGLDPQARL DMGYNSYDQV | 200 |
| TQAGKAAVAR FYGRAADKSY FIGCSEGGRE GMMLSQRFPS | 240 |
| HYDGIVAGAP GYQLPKAGIS GAWTTQSLAP AAVGLDAQGV | 280 |
| PLINKSFSDA DLHLLSQAIL GTCDALDGLA DGIVDNYRAC | 320 |
| QAAFDPATAA NPANGQALQC VGAKTADCLS PVQVTAIKRA | 360 |
| MAGPVNSAGT PLYNRWAWDA GMSGLSGTTY NQGWRSWWLG | 400 |
| SFNSSANNAQ RVSGFSARSW LVDFATPPEP MPMTQVAARM | 440 |
| MKFDFDIDPL KIWATSGQFT QSSMDWHGAT STDLAAFRDR | 480 |
| GGKMILYHGM SDAAFSALDT ADYYERLGAA MPGAAGFARL | 520 |
| FLVPGMNHCS GGPGTDRFDM LTPLVAWVER GEAPDQISAW | 560 |
| SGTPGYFGVA ARTRPLCPYP QIARYKGSGD INTEANFACA | 600 |
| APPIIQDEPQ TPA | 613 |

SEQ ID NO: 7
LENGTH: 5450
TYPE: DNA
ORGANISM: artificial
OTHER INFORMATION: pBLT-2:Ptac-LCC_MHETase plasmid gene sequence

| | |
|---|---|
| atcattcagg acgagcctca gactccagcg taactggact gaaaacaaac | 50 |
| taaagcgccc ttgtggcgct ttagttttgt tccgcggcca ccggctggct | 100 |
| cgcttcgctc ggcccgtgga caaccctgct ggacaagctg atggacaggc | 150 |
| tgcgcctgcc cacgagcttg accacaggga ttgcccaccg gctacccagc | 200 |
| cttcgaccac atacccaccg gctccaactg cgcggcctgc ggccttgccc | 250 |
| catcaatttt tttaattttc tctggggaaa agcctccggc ctgcggcctg | 300 |
| cgcgcttcgc ttgccggttg gacaccaagt ggaaggcggg tcaaggctcg | 350 |
| cgcagcgacc gcgcagcggc ttggccttga cgcgcctgga acgacccaag | 400 |
| cctatgcgag tgggggcagt cgaaggcgaa gcccgcccgc ctgccccccg | 450 |
| agcctcacgg cggcgagtgc gggggttcca agggggcagc gccaccttgg | 500 |
| gcaaggccga aggccgcgca gtcgatcaac aagccccgga ggggccactt | 550 |
| tttgccggag ggggagccgc gccgaaggcg tgggggaacc ccgcaggggt | 600 |
| gcccttcttt gggcaccaaa gaactagata tagggcgaaa tgcgaaagac | 650 |
| ttaaaaatca acaacttaaa aaaggggggt acgcaacagc tcattgcggc | 700 |
| accccccgca atagctcatt gcgtaggtta aagaaaatct gtaattgact | 750 |
| gccactttta cgcaacgcat aattgttgtc gcgctgccga aaagttgcag | 800 |
| ctgattgcgc atggtgccgc aaccgtgcgg caccctaccg catggagata | 850 |
| agcatggcca cgcagtccag agaaatcggc attcaagcca agaacaagcc | 900 |
| cggtcactgg gtgcaaacgg aacgcaaagc gcatgaggcg tgggccgggc | 950 |
| ttattgcgag gaaacccacg gcggcaatgc tgctgcatca cctcgtggcg | 1000 |
| cagatgggcc accagaacgc cgtggtggtc agccagaaga cactttccaa | 1050 |
| gctcatcgga cgttctttgc ggacggtcca atacgcagtc aaggacttgg | 1100 |
| tggccgagcg ctggatctcc gtcgtgaagc tcaacggccc cggcaccgtg | 1150 |
| tcggcctacg tggtcaatga ccgcgtggcg tggggccagc cccgcgacca | 1200 |
| gttgcgcctg tcggtgttca gtgccgccgt ggtggttgat cacgacgacc | 1250 |
| aggacgaatc gctgttgggg catggcgacc tgcgccgcat cccgaccctg | 1300 |
| tatccgggcg agcagcaact accgaccggc cccggcgagg agccgcccag | 1350 |
| ccagcccggc attccgggca tggaaccaga cctgccagcc ttgaccgaaa | 1400 |
| cggaggaatg ggaacggcgc gggcagcagc gcctgccgat gcccgatgag | 1450 |
| ccgtgttttc tggacgatgg cgagccgttg gagccgccga cacgggtcac | 1500 |
| gctgccgcgc cggtagtacg taagaggttc caactttcac cataatgaaa | 1550 |
| taagatcact accgggcgta ttttttgagt tatcgagatt ttcaggagct | 1600 |
| aaggaagcta aaatgagcca tattcaacgg gaaacgtctt gctcgaggcc | 1650 |
| gcgattaaat tccaacatgg atgctgattt atatgggtat aaatgggctc | 1700 |
| gcgataatgt cgggcaatca ggtgcgacaa tctatcgatt gtatgggaag | 1750 |
| cccgatgcgc cagagttgtt tctgaaacat ggcaaaggta gcgttgccaa | 1800 |
| tgatgttaca gatgagatgg tcaggctaaa ctggctgacg gaatttatgc | 1850 |
| ctcttccgac catcaagcat tttatccgta ctcctgatga tgcatggtta | 1900 |
| ctcaccactg cgatcccagg gaaaacagca ttccaggtat tagaagaata | 1950 |
| tcctgattca ggtgaaaata ttgttgatgc gctggcagtg ttcctgcgcc | 2000 |
| ggttgcattc gattcctgtt tgtaattgtc cttttaacgg cgatcgcgta | 2050 |
| tttcgtctcg ctcaggcgca atcacgaatg aataacggtt tggttggtgc | 2100 |
| gagtgatttt gatgacgagc gtaatggctg gcctgttgaa caagtctgga | 2150 |
| aagaaatgca taagcttttg ccattctcac cggattcagt cgtcactcat | 2200 |
| ggtgatttct cacttgataa ccttattttt gacgagggga aattaatagg | 2250 |
| ttgtattgat gttggacgag tcggaatcgc agaccgatac caggatcttg | 2300 |
| ccatcctatg gaactgcctc ggtgagtttt ctccttcatt acagaaacgg | 2350 |
| ctttttcaaa aatatggtat tgataatcct gatatgaata aattgcagtt | 2400 |
| tcacttgatg ctcgatgagt ttttctgagg gcggatcccc ctcaagtcaa | 2450 |
| aagcctccgg tcggaggctt ttgactttct gctatggagg tcaggtatga | 2500 |
| ttacactCTA GAGAGCTGTT GACAATTAAT CATCGGCTCG | 2540 |
| TATAATGTGT GGAATTGTGA GCGGATAACA ATTTCACACC | 2580 |
| ATCAAGTCAA AACACTATAT AGGAACGAAA CCATGAACTT | 2620 |
| CCCTCGCGCG TCGCGCCTGA TGCAGGCGGC GGTCCTCGGT | 2660 |
| GGTCTGATGG CAGTCAGCGC CGCGGCCACC GCTATGGACG | 2700 |
| GTGTACTGTG GCGTGTGCGC ACCGCAGCCC TGATGGCCGC | 2740 |
| CCTGCTGGCA CTGGCTGCGT GGGCGCTGGT GTGGGCGTCG | 2780 |
| CCGAGCGTTG AGGCGCAGAG CAACCCTTAC CAGCGTGGCC | 2820 |
| CGAACCCGAC CCGCTCGGCC CTGACCGCCG ACGGTCCATT | 2860 |
| CTCTGTCGCC ACCTATACCG TCAGCCGCCT GTCGGTGTCG | 2900 |
| GGTTTCGGCG GCGGCGTGAT CTACTACCCG ACCGGCACCT | 2940 |
| CCTTGACCTT CGGCGGCATT GCCATGTCCC CTGGCTACAC | 2980 |
| CGCTGACGCC TCGAGCCTGG CCTGGCTGGG TCGCCGTCTG | 3020 |
| GCCAGCCACG GCTTCGTCGT ACTGGTCATC AACACCAACA | 3060 |
| GCCGTTTCGA CGGCCCTGAC TCCCGCGCCA GCCAGCTGTC | 3100 |
| GGCTGCTCTG AACTACCTGC GTACCTCGTC GCCGTCCGCA | 3140 |
| GTCCGTGCCC GCCTGGACGC CAACCGTCTG GCCGTAGCCG | 3180 |
| GCCACTCGAT GGGCGGCGGC GGCACCCTGC GCATCGCCGA | 3220 |
| ACAAAACCCA AGCCTGAAGG CCGCTGTCCC ACTGACCCCG | 3260 |
| TGGCACACCG ACAAGACCTT CAACACCTCG GTGCCAGTGC | 3300 |
| TGATCGTAGG TGCGGAGGCC GACACCGTGG CCCCAGTGTC | 3340 |
| CCAGCACGCC ATCCCGTTCT ACCAGAACCT GCCGTCCACC | 3380 |
| ACCCCAAAGG TTTATGTCGA GCTCTGCAAC GCCAGTCACA | 3420 |
| TCGCCCCGAA TTCGAACAAC GCTGCCATCT CCGTCTACAC | 3460 |
| CATCAGCTGG ATGAAGCTGT GGGTTGACAA CGACACCCGC | 3500 |
| TACCGCCAGT TCCTGTGTAA CGTGAACGAC CCGGCCCTGT | 3540 |
| GCGATTTCCG TACCAACAAT CGTCACTGCC AGTGAcaagg | 3580 |
| attacatata agggtatata aatgcagacc accgtcacca ctatgctgct ggcatcggtc | 3640 |
| gccctggccg cctgcgcagg cggcggcagc accccgctgc cgctgccgca gcaacagccg | 3700 |
| ccacagcagg agccgccgcc tcctccagtc ccgctggctt cccgtgctgc | 3750 |
| gtgtgaggcc ctgaaggacg gcaacgggga catggtttgg ccgaacgccg | 3800 |
| ccaccgtagt tgaagtggcc gcatggcgcg acgctgcccc ggctaccgcg | 3850 |
| tccgccgccg ctctgccgga acactgcgaa gttagcggcg ccatcgccaa | 3900 |
| gcgcactggt attgacggtt atccgtacga aatcaagttc cgcctgcgca | 3950 |
| tgccggcgga gtggaatggc cgtttcttca tggagggtgg ttccggcacc | 4000 |
| aacggctccc tgagcgcggc caccggcagc atcggtggcg gccagatcgc | 4050 |
| ctcggccctg tcccgcaact tcgccaccat cgcgaccgac ggtggccacg | 4100 |
| acaacgctgt caacgacaat ccagacgccc tgggtacggt agcgttcggc | 4150 |
| ctggacccac aggctcgcct ggacatgggt tacaattcgt acgaccaggt | 4200 |
| gacccaagct ggcaaagccg ccgttgcccg tttctacggc cgtgccgccg | 4250 |
| acaagtcgta cttcatcggc tgctcggaag gtggtcggga gggcatgatg | 4300 |
| ctcagccaac gcttcccatc ccactacgac ggtatcgtcg ccggtgcccc | 4350 |
| tggctaccag ctgcctaaag ccggtatctc gggcgcttgg accactcagt | 4400 |
| cgctggcccc ggcggcggtg ggcctggacg ctcagggcgt cccgctgatc | 4450 |
| aacaagagct tctccgatgc cgacctgcac ctgctgtcgc aggccatcct | 4500 |
| cggtacttgc gatgcgctgg acggcctggc tgacggcatc gttgacaact | 4550 |
| accgcgcgtg ccaggccgct ttcgacccgg ctaccgcggc taaccctgcc | 4600 |
| aacggtcaag ctctgcaatg tgtgggtgcc aaaaccgccg attgcctgag | 4650 |
| cccggtacag gttaccgcca tcaaacgtgc aatggccggc ccggtcaaca | 4700 |
| gcgccggcac cccgctgtac aaccgttggg cctgggacgc tggtatgagc | 4750 |
| ggcctgtccg gtaccaccta caatcagggc tggcgttcct ggtggctggg | 4800 |
| tagcttcaac tcctcggcga acaacgcgca gcgtgtttcg ggtttctccg | 4850 |
| cccgctcctg gctggtcgac ttcgccaccc caccagagcc tatgccgatg | 4900 |
| acccaggtgg ctgcacgcat gatgaaattc gacttcgaca tcgacccgct | 4950 |
| gaagatctgg gccaccagcg gccagttcac ccagtcgagc atggactggc | 5000 |
| acggggccac ctccaccgac ctggccgcct tccgcgatcg tggcggcaag | 5050 |
| atgatcctgt accacggtat gagcgacgca gccttctcgg ccctggacac | 5100 |
| cgctgactac tacgaacgcc tgggcgccgc tatgccgggc gccgcgggct | 5150 |
| tcgctcgtct gttcctcgtc ccaggcatga accactgttc gggcggtcca | 5200 |
| ggtaccgacc gtttcgacat gctgacccct ctggtggcgt gggttgagcg | 5250 |
| cggcgaagcc ccggaccaga tctcggcgtg gagcggcacc ccaggctact | 5300 |
| tcggcgtcgc tgcccgtacc cgcccgctgt gcccgtaccc gcaaatcgca | 5350 |
| cgctacaagg gttccggcga tatcaacacc gaagcaaact tcGCCTGCGC | 5400 |
| CGCGCCTCCG | 5450 |

SEQ ID NO: 8
LENGTH: 1070
TYPE: DNA
ORGANISM: artificial
OTHER INFORMATION: new synthetic LCC expression cassette

| | |
|---|---|
| ctctagagag ctgttgacaa ttaatcatcg gctcgtataa tgtgtggaat | 50 |
| tgtgagcgga taacaatttc acaccatcaa gtcaaaacac tatataggaa | 100 |
| cgaaaccatg aacttccctc gcgcgtcgcg cctgatgcag gcggcggtcc | 150 |
| tcggtggtct gatggcagtc agcgccgcgg ccaccgctat ggacggtgta | 200 |
| ctgtggcgtg tgcgcaccgc agccctgatg gccgccctgc tggcactggc | 250 |
| tgcgtgggcg ctggtgtggg cgtcgccgag cgttgaggcg cagagcaacc | 300 |
| cttaccagcg tggcccgaac ccgacccgct cggccctgac cgccgacggt | 350 |
| ccattctctg tcgccaccta taccgtcagc cgcctgtcgg tgtcgggttt | 400 |
| cggcggcggc gtgatctact acccgaccgg cacctccttg accttcggcg | 450 |
| gcattgccat gtcccctggc tacaccgctg acgcctcgag cctggcctgg | 500 |
| ctgggtcgcc gtctggccag ccacggcttc gtcgtactgg tcatcaacac | 550 |
| caacagccgt ttcgacggcc ctgactcccg cgccagccag ctgtcggctg | 600 |
| ctctgaacta cctgcgtacc tcgtcgccgt ccgcagtccg tgcccgcctg | 650 |
| gacgccaacc gtctggccgt agccggccac tcgatgggcg gcggcggcac | 700 |
| cctgcgcatc gccgaacaaa acccaagcct gaaggccgct gtcccactga | 750 |
| ccccgtggca caccgacaag accttcaaca cctcggtgcc agtgctgatc | 800 |
| gtaggtgcgg aggccgacac cgtggcccca gtgtcccagc acgccatccc | 850 |
| gttctaccag aacctgccgt ccaccacccc aaaggtttat gtcgagctct | 900 |
| gcaacgccag tcacatcgcc ccgaattcga acaacgctgc catctccgtc | 950 |
| tacaccatca gctggatgaa gctgtgggtt gacaacgaca cccgctaccg | 1000 |
| ccagttcctg tgtaacgtga acgacccggc cctgtgcgat ttccgtacca | 1050 |
| acaatcgtca ctgccagtga | 1070 |

SEQ ID NO: 9
LENGTH: 4965
TYPE: DNA
ORGANISM: artificial
OTHER INFORMATION: ACE-540972 plasmid gene sequence

| | |
|---|---|
| atggccacgc agtccagaga aatcggcatt caagccaaga acaagcccgg | 50 |
| tcactgggtg caaacggaac gcaaagcgca tgaggcgtgg gccgggctta | 100 |
| ttgcgaggaa acccacggcg gcaatgctgc tgcatcacct cgtggcgcag | 150 |
| atgggccacc agaacgccgt ggtggtcagc cagaagacac tttccaagct | 200 |
| catcggacgt tctttgcgga cggtccaata cgcagtcaag gacttggtgg | 250 |
| ccgagcgctg gatctccgtc gtgaagctca acggccccgg caccgtgtcg | 300 |
| gcctacgtgg tcaatgaccg cgtggcgtgg ggccagcccc gcgaccagtt | 350 |
| gcgcctgtcg gtgttcagtg ccgccgtggt ggttgatcac gacgaccagg | 400 |
| acgaatcgct gttggggcat ggcgacctgc gccgcatccc gaccctgtat | 450 |
| ccgggcgagc agcaactacc gaccggcccc ggcgaggagc cgcccagcca | 500 |
| gcccggcatt ccgggcatgg aaccagacct gccagccttg accgaaacgg | 550 |
| aggaatggga acggcgcggg cagcagcgcc tgccgatgcc cgatgagccg | 600 |
| tgttttctgg acgatggcga gccgttggag ccgccgacac gggtcacgct | 650 |
| gccgcgccgg tagtacgtaa gaggttccaa ctttcaccat aatgaaataa | 700 |
| gatcactacc gggcgtattt tttgagttat cgagattttc aggagctaag | 750 |
| gaagctaaaa tgagccatat tcaacgggaa acgtcttgct cgaggccgcg | 800 |
| attaaattcc aacatggatg ctgatttata tgggtataaa tgggctcgcg | 850 |
| gcaatcaggt gcgacaatct atcgattgta tgggaagccc gatgcgccag | 900 |
| agttgtttct gaaacatggc aaaggtagcg ttgccaatga tgttacagat | 950 |
| gagatggtca ggctaaactg gctgacggaa tttatgcctc ttccgaccat | 1000 |
| caagcatttt atccgtactc ctgatgatgc atggttactc accactgcga | 1050 |
| tcccagggaa aacagcattc caggtattag aagaatatcc tgattcaggt | 1100 |
| gaaaatattg ttgatgcgct ggcagtgttc ctgcgccggt tgcattcgat | 1150 |
| tcctgtttgt aattgtcctt ttaacggcga tcgcgtattt cgtctcgctc | 1200 |
| aggcgcaatc acgaatgaat aacggtttgg ttggtgcgag tgattttgat | 1250 |
| gacgagcgta atggctggcc tgttgaacaa gtctggaaag aaatgcataa | 1300 |
| gcttttgcca ttctcaccgg attcagtcgt cactcatggt gatttctcac | 1350 |
| ttgataacct tatttttgac gaggggaaat taataggttg tattgatgtt | 1400 |
| ggacgagtcg gaatcgcaga ccgataccag gatcttgcca tcctatggaa | 1450 |
| ctgcctcggt gagttttctc cttcattaca gaaacggctt tttcaaaaat | 1500 |
| atggtattga taatcctgat atgaataaat tgcagtttca cttgatgctc | 1550 |
| gatgagtttt tctgagggcg gatccccctc aacggatccc cctcaagtca | 1600 |
| aaagcctccg gtcggaggct tttgactttc tgctatggag gtcaggtatg | 1650 |
| attacactCT AGAGAGCTGT TGACAATTAA TCATCGGCTC | 1690 |
| GTATAATGTG TGGAATTGTG AGCGGATAAC AATTTCACAC | 1730 |
| CATCAAGTCA AAACACTATA TAGGAACGAA ACCATGAACT | 1770 |
| TCCCTCGCGC GTCGCGCCTG ATGCAGGCGG CGGTCCTCGG | 1810 |
| TGGTCTGATG GCAGTCAGCG CCGCGGCCAC CGCTatgtgc | 1850 |
| ctgagcctgg cggcactgct gaacgcggtt ctggtgctgc gcgccgcacc | 1900 |
| ggccctgggt agcccggcaa agcgtagctg ctcccaggca gatctgaccg | 1950 |
| tggagctgga cagcggcatc gttcacggca ccgtggagag cggcaacccg | 2000 |
| gatgttcgtc agttcctggg tatcccgtac gcgaaaccgc cggtgggtga | 2050 |
| cctgcgcttc gcaccgagcg agccgattga gagcttcggc gagatcaccg | 2100 |
| cggataccct gccgccgagc tgtatgcagt acctgaccag cctggagagc | 2150 |
| atctggacca tcgacgtgct gcaattcaac gaagcgggtc tgaacaccac | 2200 |
| cggtccgcag agcgaggatt gcctgaccat cagcgtgtgg gcgccgcgtg | 2250 |
| gcgccaagaa agacctgccg gtcctgctgt ggatctacgg cggtagcttc | 2300 |
| aagaccggcg gtgaggatgt cccgtaccag atcccgaccc agtgggttca | 2350 |
| gcgtacccag gaccacatcg tggtcagctt caactaccgc gtcaacatct | 2400 |
| tcggtttccc gaacgcggcc ggtctggacg atgacaagca gaacctgggc | 2450 |
| ctgctggatc agcgtctggc ggtcgagtgg gttcgcgata acatcgccaa | 2500 |
| attcggcggt gacgttaacc gtatcggtct gtggggtcag agcgcgggcg | 2550 |
| gtatcagcgt cgcctactac agctacacct acccggagga cccgattgtg | 2600 |
| agcgcgctgc tgatgaacag cggtaacgag tacctggata tcaccagcag | 2650 |
| cgacaccacc cacagcaact tcaccttcat ggccagccag ttcggttgcg | 2700 |
| gtgatctggc cccggatgag gagctggcgt gtatgcgtac cgttgatgcg | 2750 |
| agcgccatcg aggagttcct gcacatctac atcgacaacg gcaccgagcc | 2800 |
| ggcggtgagc ttcgcaccga ttgtggacga gaagaccgtc ttcagcgatt | 2850 |
| tctacgaccg cgccgtcaac ggtagcgttg ccgcactgcc gaccatcctg | 2900 |
| ggcagcaacc tggatgacgg tgtgccgttc gtcacctaca gcccggatgg | 2950 |
| cgttaacgcg accgacgcct acgaggtgac caccgactac ttcttctgcc | 3000 |
| cggcgttcaa gagcgccaac aaccgtcacg cggccggtgc gccggtgttc | 3050 |
| cgctacgagt acagcggcaa cttcagcaac atcagcccga aaccgtggat | 3100 |
| gggtgcctgg cacaacagcg agctgccgct gctgttcggc acctacagca | 3150 |
| actaccgtgg tccgagcacc agcctggaga tcgagaccag catcgcgatg | 3200 |
| caggatgcct ggctgagcct ggtcgcgaac ggtgcggccg gtccgctggc | 3250 |
| cctgggttgg ccgctgtaca gcccggaggc gggcagcctg ctgcgcgatt | 3300 |
| toggtaaaga cgtggcggcc cagaccacca acttcgagga ctgggagagc | 3350 |
| acctgctccg aggtgttcca gccgggaggt ggaggaggtt ctatgagcaa | 3400 |
| gggcgaggag ctctttaccg gcgtcgtccc cattctcgtt gagctggacg | 3450 |
| gcgacgtgaa cggacataag ttcagtgtct cgggcgaggg cgaaggagat | 3500 |
| gccacctatg ggaagctaac cctgaagttc atctgcacaa ccgggaagct | 3550 |
| gccggtcccc tggccgacgc tggttaccac cctgacctac ggcgtgcaat | 3600 |
| gcttctcgcg ctaccctgac cacatgaagc gccacgactt cttcaaatcc | 3650 |
| gctatgccgg agggctacgt ccaggaacgc accatattct tcaaggacga | 3700 |
| cggtaactac aagacgcgcg ccgaagtcaa gttcgagggg gataccctcg | 3750 |
| tgaaccgaat cgagttgaag gggatcgact tcaaagaaga tggcaacatc | 3800 |
| ctcggccaca aactggagta caactacaat tcgcataacg tgtacatcat | 3850 |
| ggccgacaag cagaagaatg gcatcaaggt gaacttcaag attcgccaca | 3900 |
| acatcgagga cgggtccgtt cagctggccg accactatca gcagaacaca | 3950 |
| ccaattggag acggccccgt cctgctcccc gataaccatt acctttcgac | 4000 |
| acagtcggcg ctgtcgaagg acccgaacga aaagcgggac cacatggtgc | 4050 |
| tcctggagtt cgtcacggcg gccgggatca cgcacggaat ggacgaactc | 4100 |
| tacaagtagG ATATCattca ggacgagcct cagactccag cgtaactgga | 4150 |
| ctgaaaacaa actaaagcgc ccttgtggcg ctttagtttt gttccgcggc | 4200 |
| caccggctgg ctcgcttcgc tcggcccgtg gacaaccctg ctggacaagc | 4250 |
| tgatggacag gctgcgcctg cccacgagct tgaccacagg gattgcccac | 4300 |
| cggctaccca gccttcgacc acatacccac cggctccaac tgcgcggcct | 4350 |
| gcggccttgc cccatcaatt tttttaattt tctctgggga aaagcctccg | 4400 |
| gcctgcggcc tgcgcgcttc gcttgccggt tggacaccaa gtggaaggcg | 4450 |
| ggtcaaggct cgcgcagcga ccgcgcagcg gcttggcctt gacgcgcctg | 4500 |
| gaacgaccca agcctatgcg agtgggggca gtcgaaggcg aagcccgccc | 4550 |
| gcctgccccc cgagcctcac ggcggcgagt gggggttc caagggggca | 4600 |
| gcgccacctt gggcaaggcc gaaggccgcg cagtcgatca acaagccccg | 4650 |
| gaggggccac tttttgccgg agggggagcc gcgccgaagg cgtgggggaa | 4700 |
| ccccgcaggg gtgcccttct ttgggcacca aagaactaga tatagggcga | 4750 |
| aatgcgaaag acttaaaaat caacaactta aaaaaggggg gtacgcaaca | 4800 |
| gctcattgcg gcaccccccg caatagctca ttgcgtaggt taaagaaaat | 4850 |
| ctgtaattga ctgccacttt tacgcaacgc ataattgttg tcgcgctgcc | 4900 |
| gaaaagttgc agctgattgc gcatggtgcc gcaaccgtgc ggcaccctac | 4950 |
| cgcatggaga taagc | 4965 |

SEQ ID NO: 10
LENGTH: 5028
TYPE: DNA
ORGANISM: artificial
OTHER INFORMATION: LAP_117292 plasmid gene sequence

| | |
|---|---|
| atggccacgc agtccagaga aatcggcatt caagccaaga acaagcccgg | 50 |
| tcactgggtg caaacggaac gcaaagcgca tgaggcgtgg gccgggctta | 100 |
| ttgcgaggaa acccacggcg gcaatgctgc tgcatcacct cgtggcgcag | 150 |
| atgggccacc agaacgccgt ggtggtcagc cagaagacac tttccaagct | 200 |
| catcggacgt tctttgcgga cggtccaata cgcagtcaag gacttggtgg | 250 |
| ccgagcgctg gatctccgtc gtgaagctca acggccccgg caccgtgtcg | 300 |
| gcctacgtgg tcaatgaccg cgtggcgtgg ggccagcccc gcgaccagtt | 350 |
| gcgcctgtcg gtgttcagtg ccgccgtggt ggttgatcac gacgaccagg | 400 |
| acgaatcgct gttggggcat ggcgacctgc gccgcatccc gaccctgtat | 450 |
| ccgggcgagc agcaactacc gaccggcccc ggcgaggagc cgcccagcca | 500 |
| gcccggcatt ccgggcatgg aaccagacct gccagccttg accgaaacgg | 550 |
| aggaatggga acggcgcggg cagcagcgcc tgccgatgcc cgatgagccg | 600 |
| tgttttctgg acgatggcga gccgttggag ccgccgacac gggtcacgct | 650 |
| gccgcgccgg tagtacgtaa gaggttccaa ctttcaccat aatgaaataa | 700 |
| gatcactacc gggcgtattt tttgagttat cgagattttc aggagctaag | 750 |
| gaagctaaaa tgagccatat tcaacgggaa acgtcttgct cgaggccgcg | 800 |
| attaaattcc aacatggatg ctgatttata tgggtataaa tgggctcgcg | 850 |
| ataatgtcgg gcaatcaggt gcgacaatct atcgattgta tgggaagccc | 900 |
| gatgcgccag agttgtttct gaaacatggc aaaggtagcg ttgccaatga | 950 |
| tgttacagat gagatggtca ggctaaactg gctgacggaa tttatgcctc | 1000 |
| ttccgaccat caagcatttt atccgtactc ctgatgatgc atggttactc | 1050 |
| accactgcga tcccagggaa aacagcattc caggtattag aagaatatcc | 1100 |
| tgattcaggt gaaaatattg ttgatgcgct ggcagtgttc ctgcgccggt | 1150 |
| tgcattcgat tcctgtttgt aattgtcctt ttaacggcga tcgcgtattt | 1200 |
| cgtctcgctc aggcgcaatc acgaatgaat aacggtttgg ttggtgcgag | 1250 |
| tgattttgat gacgagcgta atggctggcc tgttgaacaa gtctggaaag | 1300 |
| aaatgcataa gcttttgcca ttctcaccgg attcagtcgt cactcatggt | 1350 |
| gatttctcac ttgataacct tatttttgac gaggggaaat taataggttg | 1400 |
| tattgatgtt ggacgagtcg gaatcgcaga ccgataccag gatcttgcca | 1450 |
| tcctatggaa ctgcctcggt gagttttctc cttcattaca gaaacggctt | 1500 |
| tttcaaaaat atggtattga taatcctgat atgaataaat tgcagtttca | 1550 |
| cttgatgctc gatgagtttt tctgagggcg gatccccctc aacggatccc | 1600 |
| cctcaagtca aaagcctccg gtcggaggct tttgactttc tgctatggag | 1650 |
| gtcaggtatg attacactCT AGAGAGCTGT TGACAATTAA | 1690 |
| TCATCGGCTC GTATAATGTG TGGAATTGTG AGCGGATAAC | 1730 |
| AATTTCACAC CATCAAGTCA AAACACTATA TAGGAACGAA | 1770 |
| ACCATGAACT TCCCTCGCGC GTCGCGCCTG ATGCAGGCGG | 1810 |
| CGGTCCTCGG TGGTCTGATG GCAGTCAGCG CCGCGGCCAC | 1850 |
| CGCTatgcgt gtcaccctgg gtctggcggc cctggccgtt ggtgccagcg | 1900 |
| ccatcccgac cgagctgcgt gagcgtcagg acggccacaa cagcaaaccg | 1950 |
| tgcccgaaga aaccgctggt gaccaaagac gccctggaga aggatatcac | 2000 |
| catcaagaaa ctgatgcgcg gtgcgcagca gctggaggat ttogcctaca | 2050 |
| gctacccggc gcgtaaccgc atcatgggcg gtcaggcgca caacgacacc | 2100 |
| gtcaagtggc tgaagaaaga gctggagagc accagctact acgatgtgac | 2150 |
| cctgcaaccg ttcagcaact acgtcatgct gaacggcacc ctgaacgcct | 2200 |
| tcaccatcga cggtcgtgcg atcaacagca ccatcctgga gtacagcgcc | 2250 |
| agcaccccgg gtctggttac cgccccgatt gtgccggtga acaacctggg | 2300 |
| ttgcgaggcc agcgatttcc cggccgaggt gtccggtgcc atcgcgctga | 2350 |
| tcagccgtgg cacctgcgag ttcggtctga aaagcgccct ggcgggtagc | 2400 |
| gccggcgccg ttggtgccat catctacaac aacgtggccg gcaccatcag | 2450 |
| cgccaccctg ggcccgccgc cgcgtccgga gggtgactac gtggcgagcg | 2500 |
| tcaccctgag cctggaggaa ggccaggcca tcgttgccaa agtggccggc | 2550 |
| ggtgcgaccg tgaccggcac cctggacgtc ctgaccgatg tccaggttat | 2600 |
| caccaccaac aacgtcctgg cgaccagcaa atgcggcgac aagaacaacc | 2650 |
| agctgtggct gggtgcccac accgatagcg ttggtgccgg tccgggtatc | 2700 |
| aacgacgatg gtagcggcac cgttgccatc ctgaacgtgg ccaaaagcct | 2750 |
| ggcgaagtac aacgtgaaca acgcggtcag cttcggtttc tggagcggtg | 2800 |
| aagagagcgg tctgctgggt agcaccttct tcgtggagag cctgagcccg | 2850 |
| gaggccgccc tggacgttcg tgcctacctg aacttcgata tgatcgcgag | 2900 |
| cccgaactac gtgcaccaga tctacgatgg tgatggcagc gcctacggcc | 2950 |
| tgagcggtcc ggcgggcagc gatgatatcg aggccttctt cgcgggttac | 3000 |
| ttcaaagacc tgaacatccc gagcaacgag accgagttca acggtcgcag | 3050 |
| cgactacggc ccgttcctgg atgcaaacat cccggcgggc ggcaccacca | 3100 |
| ccggtgcaga tgaggtcaag accgttgaag agcaggccat ctggggcggt | 3150 |
| gtggccggcg agatcctgga ccagaactac caccaggcgg ccgataacgt | 3200 |
| caccaacctg aacgaggaag cgtggctgct gcacagccgc ggcatcgcgg | 3250 |
| cggcggtggc ccactacgcc accagctggg aaggcatccc ggagcgtgtt | 3300 |
| ccggcaaacg gcaccgccaa acgtagcgtg cgtgagccgg tggtccgtaa | 3350 |
| gggtgtcaaa ggcggtaaag ttaccctgaa gctgctggga ggtggaggag | 3400 |
| gttctatgag caagggcgag gagctcttta ccggcgtcgt ccccattctc | 3450 |
| gttgagctgg acggcgacgt gaacggacat aagttcagtg tctcgggcga | 3500 |
| gggcgaagga gatgccacct atgggaagct aaccctgaag ttcatctgca | 3550 |
| caaccgggaa gctgccggtc ccctggccga cgctggttac caccctgacc | 3600 |
| tacggcgtgc aatgcttctc gcgctaccct gaccacatga agcgccacga | 3650 |
| cttcttcaaa tccgctatgc cggagggcta cgtccaggaa cgcaccatat | 3700 |
| tcttcaagga cgacggtaac tacaagacgc gcgccgaagt caagttcgag | 3800 |
| ggggataccc tcgtgaaccg aatcgagttg aaggggatcg acttcaaaga | 3850 |
| agatggcaac atcctcggcc acaaactgga gtacaactac aattcgcata | 3900 |
| acgtgtacat catggccgac aagcagaaga atggcatcaa ggtgaacttc | 3950 |
| aagattcgcc acaacatcga ggacgggtcc gttcagctgg ccgaccacta | 4000 |
| tcagcagaac acaccaattg gagacggccc cgtcctgctc cccgataacc | 4050 |
| attacctttc gacacagtcg gcgctgtcga aggacccgaa cgaaaagcgg | 4100 |
| gaccacatgg tgctcctgga gttcgtcacg gcggccggga tcacgcacgg | 4150 |
| aatggacgaa ctctacaagt agGATATCat tcaggacgag cctcagactc | 4200 |
| cagcgtaact ggactgaaaa caaactaaag cgcccttgtg gcgctttagt | 4250 |
| tttgttccgc ggccaccggc tggctcgctt cgctcggccc gtggacaacc | 4300 |
| ctgctggaca agctgatgga caggctgcgc ctgcccacga gcttgaccac | 4350 |
| agggattgcc caccggctac ccagccttcg accacatacc caccggctcc | 4400 |
| aactgcgcgg cctgcggcct tgccccatca atttttttaa ttttctctgg | 4450 |
| ggaaaagcct ccggcctgcg gcctgcgcgc ttcgcttgcc ggttggacac | 4500 |
| caagtggaag gcgggtcaag gctcgcgcag cgaccgcgca gcggcttggc | 4550 |
| cttgacgcgc ctggaacgac ccaagcctat gcgagtgggg gcagtcgaag | 4600 |
| gcgaagcccg cccgcctgcc ccccgagcct cacggcggcg agtgcggggg | 4650 |
| ttccaagggg gcagcgccac cttgggcaag gccgaaggcc gcgcagtcga | 4700 |
| tcaacaagcc ccggaggggc cactttttgc cggaggggga gccgcgccga | 4750 |
| aggcgtgggg gaaccccgca ggggtgccct tctttgggca ccaaagaact | 4800 |
| agatataggg cgaaatgcga aagacttaaa aatcaacaac ttaaaaaagg | 4850 |
| ggggtacgca acagctcatt gcggcacccc ccgcaatagc tcattgcgta | 4900 |
| ggttaaagaa aatctgtaat tgactgccac ttttacgcaa cgcataattg | 4950 |
| ttgtcgcgct gccgaaaagt tgcagctgat tgcgcatggt gccgcaaccg | 5000 |
| tgcggcaccc taccgcatgg agataagc | 5028 |

SEQ ID NO: 11
Length:
Type: DNA
Organism: Artificial
Other information: pBLT-2-LCC-Mle046 plasmid gene sequence
gtgtggccaataagctttgactggactgaaaacaaactaaagcgcccttgtggcgctttagttttgttccgcggccaccggctggctcgc
ttcgctcggcccgtggacaaccctgctggacaagctgatggacaggctgcgcctgcccacgagcttgaccacagggattgcccacc
ggctacccagccttcgaccacatacccaccggctccaactgcgcggcctgcggccttgccccatcaatttttttaattttctctggggaaa
agcctccggcctgcggcctgcgcgcttcgcttgccggttggacaccaagtggaaggcgggtcaaggctcgcgcagcgaccgcgca
gcggcttggccttgacgcgcctggaacgacccaagcctatgcgagtgggggcagtcgaaggcgaagcccgcccgcctgcccccc
gagcctcacggcggcgagtgcgggggttccaagggggcagcgccaccttgggcaaggccgaaggccgcgcagtcgatcaacaa
gccccggaggggccactttttgccggagggggagccgcgccgaaggcgtgggggaaccccgcaggggtgcccttctttgggcac
caaagaactagatatagggcgaaatgcgaaagacttaaaaatcaacaacttaaaaaaggggggtacgcaacagctcattgcggcacc
ccccgcaatagctcattgcgtaggttaaagaaaatctgtaattgactgccacttttacgcaacgcataattgttgtcgcgctgccgaaaag
ttgcagctgattgcgcatggtgccgcaaccgtgcggcaccctaccgcatggagataagcatggccacgcagtccagagaaatcggc
attcaagccaagaacaagcccggtcactgggtgcaaacggaacgcaaagcgcatgaggcgtgggccgggcttattgcgaggaaac
ccacggcggcaatgctgctgcatcacctcgtggcgcagatgggccaccagaacgccgtggtggtcagccagaagacactttccaag
ctcatcggacgttctttgcggacggtccaatacgcagtcaaggacttggtggccgagcgctggatctccgtcgtgaagctcaacggcc
ccggcaccgtgtcggcctacgtggtcaatgaccgcgtggcgtggggccagccccgcgaccagttgcgcctgtcggtgttcagtgcc
gccgtggtggttgatcacgacgaccaggacgaatcgctgttggggcatggcgacctgcgccgcatcccgaccctgtatccgggcga
gcagcaactaccgaccggccccggcgaggagccgcccagccagcccggcattccgggcatggaaccagacctgccagccttgac
cgaaacggaggaatgggaacggcgcgggcagcagcgcctgccgatgcccgatgagccgtgttttctggacgatggcgagccgttg
gagccgccgacacgggtcacgctgccgcgccggtagtacgtaagaggttccaactttcaccataatgaaataagatcactaccgggc
gtattttttgagttatcgagattttcaggagctaaggaagctaaaatgagccatattcaacgggaaacgtcttgctcgaggccgcgattaa
attccaacatggatgctgatttatatgggtataaatgggctcgcgataatgtcgggcaatcaggtgcgacaatctatcgattgtatgggaa
gcccgatgcgccagagttgtttctgaaacatggcaaaggtagcgttgccaatgatgttacagatgagatggtcaggctaaactggctga
cggaatttatgcctcttccgaccatcaagcattttatccgtactcctgatgatgcatggttactcaccactgcgatcccagggaaaacagc
attccaggtattagaagaatatcctgattcaggtgaaaatattgttgatgcgctggcagtgttcctgcgccggttgcattcgattcctgtttgt
aattgtccttttaacggcgatcgcgtatttcgtctcgctcaggcgcaatcacgaatgaataacggtttggttggtgcgagtgattttgatga
cgagcgtaatggctggcctgttgaacaagtctggaaagaaatgcataagcttttgccattctcaccggattcagtcgtcactcatggtgat
ttctcacttgataaccttatttttgacgaggggaaattaataggttgtattgatgttggacgagtcggaatcgcagaccgataccaggatctt
gccatcctatggaactgcctcggtgagttttctccttcattacagaaacggctttttcaaaaatatggtattgataatcctgatatgaataaat
tgcagtttcacttgatgctcgatgagtttttctgagggoggatccccctcaagtcaaaagcctccggtcggaggcttttgactttctgctatg
gaggtcaggtatgattacactCTAGAGAGCTGTTGACAATTAATCATCGGCTCGTATAATGTG
TGGAATTGTGAGCGGATAACAATTTCACACCATCAAGTCAAAACACTATATAGG
AACGAAACCATGAACTTCCCTCGCGCGTCGCGCCTGATGCAGGCGGCGGTCCTC
GGTGGTCTGATGGCAGTCAGCGCCGCGGCCACCGCTATGGACGGTGTACTGTGG
CGTGTGCGCACCGCAGCCCTGATGGCCGCCCTGCTGGCACTGGCTGCGTGGGCGC
TGGTGTGGGCGTCGCCGAGCGTTGAGGCGCAGAGCAACCCTTACCAGCGTGGCC
CGAACCCGACCCGCTCGGCCCTGACCGCCGACGGTCCATTCTCTGTCGCCACCTA
TACCGTCAGCCGCCTGTCGGTGTCGGGTTTCGGCGGCGGCGTGATCTACTACCCG
ACCGGCACCTCCTTGACCTTCGGCGGCATTGCCATGTCCCCTGGCTACACCGCTG
ACGCCTCGAGCCTGGCCTGGCTGGGTCGCCGTCTGGCCAGCCACGGCTTCGTCGT
ACTGGTCATCAACACCAACAGCCGTTTCGACGGCCCTGACTCCCGCGCCAGCCAG
CTGTCGGCTGCTCTGAACTACCTGCGTACCTCGTCGCCGTCCGCAGTCCGTGCCC
GCCTGGACGCCAACCGTCTGGCCGTAGCCGGCCACTCGATGGGCGGCGGCGGCA
CCCTGCGCATCGCCGAACAAAACCCAAGCCTGAAGGCCGCTGTCCCACTGACCC
CGTGGCACACCGACAAGACCTTCAACACCTCGGTGCCAGTGCTGATCGTAGGTG
CGGAGGCCGACACCGTGGCCCCAGTGTCCCAGCACGCCATCCCGTTCTACCAGA
ACCTGCCGTCCACCACCCCAAAGGTTTATGTCGAGCTCTGCAACGCCAGTCACAT
CGCCCCGAATTCGAACAACGCTGCCATCTCCGTCTACACCATCAGCTGGATGAAG
CTGTGGGTTGACAACGACACCCGCTACCGCCAGTTCCTGTGTAACGTGAACGACC
CGGCCCTGTGCGATTTCCGTACCAACAATCGTCACTGCCAGTGAcaaggattacatataagg
gtatataaatgAACTTCCCtcgcgcgtcgcgcctgatgcaggcggcggtcctcggtggtctgatggcagtcagcgccgcggc
caccgctcatatggcagttgcaggtgacttcccgaatcagttcctgagctgcttcgatgcagccaatctgaccgaaattgaactgccgg
cagatgttcagggcttccgtctgattgaaattgccgaacatgccggtgataaaggcatgccggcccattgtgaaattgtgggcgccatta
atgatcgcattagtccggtggatggccagcattatagcattaaattccgtctgcgtctgccgcaggattggaatggtcgcttctatatgga
aggcggtggcggtagcaatggtgttctgaaagatgcaatgggcccgaccggcctgaatcaggaagatagtgccctggaacgcggct
tcgcagtggtgaccaccgatagcggccatgataatgataccaatagcgatagcaatgccagcggccgcagcgccttcggtatggatc
cgcaggcacgcctggacttcggttatatgagttatgatattgttacccgtgtgggtaaagcaattgtggaaaaatattatggtgcagcccc
ggaaaaaagttacttcattggttgtagtgaaggcggtcgtgaagcagccctgatgacccagcgctatccggatctgtatgatggtgtggt
tgccggtgccccgggtattcacttcagctatagtgcagcctatgccccgttcctgctgcgcatcttcggtaatctggccgaaaccagaaa
tcagagcggtccggatggtattccgctgctgaataaactgtatagtgataatgatgtgcagctgattgccgatgccgttgtgggcgcctg
tgatgccctggatggcctggaagatcgtatgagcaataatattgaagcctgtaccaccgttaccgtgctgccgcgcctgcgcgctctga
catgcagtggcgccaaagaagaaggttgcctgctggaagatcagattgatgccttcgtggccggtatggcaggtccggtgaccagtg
atggtacccgtctgtatccgggtcatccgtgggatccgggtattggcggtcgtattggtgatagtgttaatgatggcttccgtagttggtg
gttcggtagctatgatagcgatcagaataatgcccgcaaagtgaccctgagcaccccgcagcatgccatgctgtggcagaccccgcc
ggttccgctgcgtcctgatgaatatgtgcgcttcgaaatgaacttcaatattgatgaaacacctgccctggcatacgctaccaccgatctg
tatccggtgagcagtgcagaactgggcaatgccgatagtccggatctgagcgacttcgcaagccgcggcggtaaactggtgatctat
catggtgccgcagatgcagcattcagtgcactggataccattaaatattggaatgccgttaatgaaaccgccgatggtcaggccgcag
acttcgcacgtctgttcattattccgggtatgaatcattgccagggtggtccggccaccgatgatgttgatctgctgaccccgctgatggc
atgggttgaagatgatctgccgattgaacgcctggaagccaccgtgagcaatccggattacttcggtggtaaaaatctgagtcgcccg
ctgtgtccgtatccgctgtatgccgaatatgatggtgaaggtgatccgagtagcgcagaatcattcacct
SEQ ID NO: 12
Length:
Type: DNA
Organism: Artificial
Other information: Mutant Mle046 DNA sequence
Ggatccccctcaagtcaaaagcctccggtcggaggcttttgactttctgctatggaggtcaggtatgattttgcattaggcaccccaggc
ttgacaattaatcatcggctcgtataatgtgtggaattgtgagcggataacaatttcacactctagaggaggaatcttactctagaatgcat
atggcagttgcaggtgacttcccgaatcagttcctgagctgcttcgatgcagccaatctgaccgaaattgaactgccggcagatgttca
gggcttccgtctgattgaaattgccgaacatgccggtgataaaggcatgccggcccattgtgaaattgtgggcgccattaatgatcgcat
tagtccggtggatggccagcattatagcattaaattccgtctgcgtctgccgcaggattggaatggtcgcttctatatggaaggcggtgg
cggtagcaatggtgttctgaaagatgcaatgggcccgaccggCCTGAATCAGGAAGATAGTGCCCTGGAA
CGCAGTTTCGCAGTGGtgaccaccgatagcggccatgataatgataccaatagcgatagcaatgccagcggccgcag
cgccttcggtatggatccgcaggcacgcctggacttcggttatatgagttatgatattgttacccgtgtgggtaaagcaattgtggaaaaa
tattatggtgcagccccggaaaaaagttacttcattggttgtagtgaaggcggtcgtgaagcagccctgatgacccagcgctatccgga
tctgtatgatggtgtggttgccggtgccccgggtattcacttcagctatagtgcagcctatgccccgttcctgctgcgcatcttcggtaatc
tggccgaaaccagaaatcagagcggtccggatggtattccgctgctgaataaactgtatagtgataatgatgtgcagctgattgccgat
gccgttgtgggcgcctgtgatgccctggatggcctggaagatcgtatgagcaataatattgaagcctgtaccaccgttaccgtgctgcc
gcgcctgcgcgctctgacatgcagtggcgccaaagaagaaggttgcctgctggaagatcagattgatgccttcgtggccggtatggc
aggtccggtgaccagtgatggtacccgtctgtatccgggtcatccgtgggatccgggtattggcggtcgtattggtgatagtgttaatga
tggcttccgtagttggtggttcggtagctatgatagcgatcagaataatgcccgcaaagtgaccctgagcaccccgcagcatgccatgc
tgtggcagaccccgccggttccgctgcgtcctgatgaatatgtgcgcttcgaaatgaacttcaatattgatgaaacacctgccctggcat
acgctaccaccgatctgtatccggtgagcagtgcagaactgggcaatgccgatagtccggatctgagcgacttcgcaagccgcggc
ggtaaactggtgatctatcatggtgccgcagatgcagcattcagtgcactggataccattaaatattggaatgccgttaatgaaaccgcc
gatggtcaggccgcagacttcgcacgtctgttcattattccgggtatgaatcattgccagggtggtccggccaccgatgatgttgatctg
ctgaccccgctgatggcatgggttgaagatgatctgccgattgaacgcctggaagccaccgtgagcaatccggattacttcggtggta
aaaatctgagtcgcccgctgtgtccgtatccgctgtatgccgaatatgatggtgaaggtgatccgagtagcgcagaatcattcacctgtg
tggccaataagctttgagatatcgatatcattcaggacgagcctcagactccagcgtaactggactgaaaacaaactaaagcgcccttg
tggcgctttagttttgttcc
SEQ ID NO: 13
Length:
Type: Protein
Organism: bacteria
Other information: Mle046 protein sequence
MHMAVAGDFPNQFLSCFDAANLTEIELPADVQGFRLIEIAEHAGDKGMPAHCEIVGAINDRISPVDGQH
YSIKFRLRLPQDWNGRFYMEGGGGSNGVLKDAMGPTGLNQEDSALERGFAVVTTDSGHDNDTNSDS
NASGRSAFGMDPQARLDFGYMSYDIVTRVGKAIVEKYYGAAPEKSYFIGCSEGGREAALMTQRYPDL
YDGVVAGAPGIHFSYSAAYAPFLLRIFGNLAETRNQSGPDGIPLLNKLYSDNDVQLIADAVVGACDAL
DGLEDRMSNNIEACTTVTVLPRLRALTCSGAKEEGCLLEDQIDAFVAGMAGPVTSDGTRLYPGHPWDP
GIGGRIGDSVNDGFRSWWFGSYDSDQNNARKVTLSTPQHAMLWQTPPVPLRPDEYVRFEMNFNIDETP
ALAYATTDLYPVSSAELGNADSPDLSDFASRGGKLVIYHGAADAAFSALDTIKYWNAVNETADGQAA
DFARLFIIPGMNHCQGGPATDDVDLLTPLMAWVEDDLPIERLEATVSNPDYFGGKNLSRPLCPYPLYAE
YDGEGDPSSAESFTCVANKL*
SEQ ID NO: 14
Length:
Type: Protein
Organism: artificial
Other information: mutant Mle046 (G117S) protein sequence
MHMAVAGDFPNQFLSCFDAANLTEIELPADVQGFRLIEIAEHAGDKGMPAHCEIVGAINDRISPVDGQH
YSIKFRLRLPQDWNGRFYMEGGGGSNGVLKDAMGPTGLNQEDSALERSFAVVTTDSGHDNDTNSDSN
ASGRSAFGMDPQARLDFGYMSYDIVTRVGKAIVEKYYGAAPEKSYFIGCSEGGREAALMTQRYPDLY
DGVVAGAPGIHFSYSAAYAPFLLRIFGNLAETRNQSGPDGIPLLNKLYSDNDVQLIADAVVGACDALD
GLEDRMSNNIEACTTVTVLPRLRALTCSGAKEEGCLLEDQIDAFVAGMAGPVTSDGTRLYPGHPWDPG
IGGRIGDSVNDGFRSWWFGSYDSDQNNARKVTLSTPQHAMLWQTPPVPLRPDEYVRFEMNFNIDETPA
LAYATTDLYPVSSAELGNADSPDLSDFASRGGKLVIYHGAADAAFSALDTIKYWNAVNETADGQAAD
FARLFIIPGMNHCQGGPATDDVDLLTPLMAWVEDDLPIERLEATVSNPDYFGGKNLSRPLCPYPLYAEY
DGEGDPSSAESFTCVANKL*

### REFERENCES

Bentley, G. J.; Narayanan, N.; Jha, R. K.; Salvachna, D.; Elmore, J. R.; Peabody, G. L.; Black, B. A.; Ramirez, K.; De Capite, A.; Michener, W. E., Engineering glucose metabolism for enhanced muconic acid production in Pseudomonas putida KT2440. Metabolic engineering 2020, 59, 64-75.
Bhaskar, T.; Kaneko, J.; Muto, A.; Sakata, Y.; Jakab, E.; Matsui, T.; Uddin, M. A., Pyrolysis studies of PP/PE/PS/PVC/HIPS-Br plastics mixed with PET and dehalogenation (Br, Cl) of the liquid products. Journal of analytical and applied pyrolysis 2004, 72 (1), 27-33.
Jayakody, L. N.; Dissanayake, L., Engineering microbes to bio-upcycle polyethylene terephthalate. Frontiers in Bioengineering and Biotechnology 2021, 9, 356.
R. K. Jha, J. M. Bingen, C. W. Johnson, T. L. Kern, P. Khanna, D. S. Trettel, C. E. Strauss, G. T. Beckham, T. Dale, A protocatechuate biosensor for Pseudomonas putida KT2440 via promoter and protein evolution, Metabolic engineering communications, 6 (2018) 33-38.
Johnson, C. W.; Salvachna, D.; Rorrer, N. A.; Black, B. A.; Vardon, D. R.; John, P. C. S.; Cleveland, N. S.; Dominick, G.; Elmore, J. R.; Grundl, N., Innovative chemicals and materials from bacterial aromatic catabolic pathways. Joule 2019, 3 (6), 1523-1537.
Kenny, S. T.; Runic, J. N.; Kaminsky, W.; Woods, T.; Babu, R. P.; Keely, C. M.; Blau, W.; O'Connor, K. E., Up-cycling of PET (polyethylene terephthalate) to the biodegradable plastic PHA (polyhydroxyalkanoate). Environmental science & technology 2008, 42 (20), 7696-7701.
Leavitt, J. M.; Wagner, J. M.; Tu, C. C.; Tong, A.; Liu, Y.; Alper, H. S., Biosensor-enabled directed evolution to improve muconic acid production in Saccharomyces cerevisiae. Biotechnology journal 2017, 12 (10), 1600687.
Shojaei, B.; Abtahi, M.; Najafi, M., Chemical recycling of PET: A stepping-stone toward sustainability. Polymers for Advanced Technologies 2020, 31 (12), 2912-2938.
Tiso, T.; Narancic, T.; Wei, R.; Pollet, E.; Beagan, N.; Schroder, K.; Honak, A.; Jiang, M.; Kenny, S.; Wierckx, N., Bio-upcycling of polyethylene terephthalate. BioRxiv 2020.
Tournier, V.; Topham, C.; Gilles, A.; David, B.; Folgoas, C.; Moya-Leclair, E.; Kamionka, E.; Desrousseaux, M.-L.; Texier, H.; Gavalda, S., An engineered PET depolymerase to break down and recycle plastic bottles. Nature 2020, 580 (7802), 216-219.
Walker, T. W.; Frelka, N.; Shen, Z.; Chew, A. K.; Banick, J.; Grey, S.; Kim, M. S.; Dumesic, J. A.; Van Lehn, R. C.; Huber, G. W., Recycling of multilayer plastic packaging materials by solvent-targeted recovery and precipitation. Science advances 2020, 6 (47), eaba7599.
Sanders, M. M. 2017. The Application of Oxidative Hydrothermal Dissolution (OHD) to Organic-Rich Shales. Southern Illinois University at Carbondale.
1. Tournier, V.; Topham, C.; Gilles, A.; David, B.; Folgoas, C.; Moya-Leclair, E.; Kamionka, E.; Desrousseaux, M.-L.; Texier, H.; Gavalda, S., An engineered PET depolymerase to break down and recycle plastic bottles. Nature 2020, 580 (7802), 216-219.
2. Meyer-Cifuentes, I. E.; Öztürk, B., Mle046 is a marine mesophilic MHETase-like enzyme. Frontiers in Microbiology 2021, 12.
3. Knott, B. C.; Erickson, E.; Allen, M. D.; Gado, J. E.; Graham, R.; Kearns, F. L.; Pardo, I.; Topuzlu, E.; Anderson, J. J.; Austin, H. P., Characterization and engineering of a two-enzyme system for plastics depolymerization. Proceedings of the National Academy of Sciences 2020, 117 (41), 25476-25485.
4. Mrigwani, A.; Thakur, B.; Guptasarma, P., Enhancing high-temperature degradation of polyethylene terephthalate through a synergistic division of enzyme labour between a solid-degrading thermostable cutinase and a reaction intermediate-degrading thermostable carboxylesterase. bioRxiv 2022.

1. A recombinant bacterial cell comprising a heterologous DNA encoding and expressing at least one heterologous PETase-like enzyme and at least one heterologous MHETase-like enzyme,
wherein the PETase-like enzyme has a secretion signal peptide linked in frame to an enzymatic activity for degrading Bis(2-hydroxyethyl) terephthalate (BHET) into Mono-(2-hydroxyethyl)terephthalic acid (MHET) and comprises Leaf-Compost Cutinase (LCC) enzyme with SEQ ID NO: 5, or a functional variant thereof having at least 85% overall sequence identity to SEQ ID NO: 5; and
wherein the MHETase-like enzyme has a secretion signal peptide linked in frame to an enzymatic activity for degrading MHET into ethylene glycol and terephthalic acid and comprises a polypeptide with SEQ ID NO: 6, a functional variant thereof having at least 85% overall sequence identity to SEQ ID NO: 6, Mle046 enzyme with SEQ ID NO: 13, a functional variant thereof having at least 85% overall sequence identity to SEQ ID NO: 13, Mle046 mutant enzyme with SEQ ID NO: 14, a functional variant thereof having at least 85% overall sequence identity to SEQ ID NO: 14, or any combination thereof.

2. The recombinant bacterial cell according to claim 1, wherein the recombinant bacterial cell is Pseudomonas putida or Erwinia aphidicola.

3. The recombinant bacterial cell according to claim 1, wherein the PETase-like enzyme and/or the MHETase-like enzyme is thermostable and enzymatically active at a temperature ranging from about 30° C. to about 75° C. and/or at a pH ranging from about 6 to about 9.

4. The recombinant bacterial cell according to claim 1, wherein the PETase-like enzyme and/or the MHETase-like enzyme is expressed from an inducible promoter.

5. The recombinant bacterial cell according to claim 1, wherein the PETase-like enzyme and the MHETase-like enzyme are encoded by a plasmid and co-expressed from a single promoter.

6. The recombinant bacterial cell according to claim 1, wherein the recombinant bacterial cell contains a mutation in its genome, the mutation eliminating expression of muconate cycloisomerase enzymatic activity with SEQ ID NO: 4, wherein the mutation is a deletion and/or insertion of at least one nucleotide or more.

7. The recombinant bacterial cell according to claim 1, wherein the PETase-like enzyme and the MHETase-like enzyme are enzymatically active at 30° C., degrading bis(2-hydroxyethyl) terephthalate (BHET) into ethylene glycol and terephthalic acid.

8. The recombinant bacterial cell according to claim 1, wherein the bacterial cell is capable of growing on a substrate containing biomass co-mixed with polyethylene terephthalate (PET) pretreated in oxidative hydrothermal dissolution (OHD) process.

9. The recombinant bacterial cell according to claim 8, wherein the biomass contains green tea waste, black tea waste, used green tea, used black tea, corn stover and/or coffee brewing waste; and/or
wherein the substrate contains the biomass and PET in the following ratio by weight from 1:99 wt % to 50:50 wt % of PET to the biomass.

10. A method for producing an enzymatic composition for biodegradation of plastic material and/or biomass waste, the method comprising:
culturing the recombinant bacterial cell accordingly to claim 1 in a liquid medium, wherein the recombinant bacterial cell secrets the PETase-like enzyme and the MHETase enzyme into the liquid medium; and
collecting the liquid medium containing the PETase-like enzyme and the MHETase-like enzyme.

11. The method of claim 10, wherein the method further comprises:
centrifuging a bacterial culture and producing a supernatant and a pellet; and
collecting the supernatant containing the PETase-like enzyme and the MHETase-like enzyme.

12. A method for decomposing a plastic material containing PET (polyethylene terephthalate) or PBAT (poly(butylene adipate-co-terephthalate), the method comprising:
contacting the plastic material with the recombinant bacterial cell according to claim 1 and/or an enzymatic composition comprising at least one PETase-like enzyme and at least one MHETase-like enzyme, the enzymatic composition being produced or producible by the recombinant bacterial cell.

13. The method of claim 12, wherein the method further comprises prior to contacting the plastic material with the recombinant bacterial cell, co-mixing the plastic material with a biomass and processing the co-mixture by oxidative hydrothermal dissolution (OHD) in the presence of oxygen in subcritical water at a temperature in the range 100-374° C. and a pressure in the range 1500 to 3500 psi.

14. The method of claim 13, wherein the biomass and PET are present in a ratio by weight ranging from 1:99 wt % to 50:50 wt % of PET to the biomass.

15. The method of claim 13, wherein the plastic material is contacted at ambient temperature and a pH in the range from about 6 to about 9.

16. A method for converting a biomass into carbon-containing substrate for synthesizing polymeric products, the method comprising treating the biomass in a hydrothermal dissolution (OHD) process in the presence of oxygen in subcritical water at a temperature in the range 100-374° C. and a pressure in the range 1500 to 3500 psi, and contacting the OHD-treated biomass with a recombinant bacterium according to claim 1, or an enzymatic composition comprising at least one PETase-like enzyme and at least one MHETase-like enzyme, the enzymatic composition being produced or being producible by the recombinant bacterial cell.

17. The method of claim 16, wherein the biomass includes tea waste, coffee waste and/or corn stover.

18. A dual enzyme composition comprising at least one PETase-like enzyme and at least one MHETase-like enzyme, the composition being produced and secreted by the recombinant bacterial cell or being producible by the recombinant bacterial cell according to claim 1.

19. A dual enzyme composition comprising at least one PETase-like enzyme and at least one MHETase-like enzyme,
wherein the PETase-like enzyme comprises at least amino acids 28-320 of the polypeptide with SEQ ID NO: 5 and/or a functional variant therefore having at least 80% overall sequence identity to amino acids 28-320 of SEQ ID NO: 5, or any combination thereof, wherein the PETase-like enzyme having an enzymatic activity for degrading bis(2-hydroxyethyl) terephthalate (BHET) to mono-(2-hydroxyethyl)terephthalic acid (MHET); and
wherein the MHETase-like enzyme comprises at least amino acids 18-613 of the polypeptide with SEQ ID NO: 6 and/or a functional variant thereof having at least 80% overall sequence identity to amino acids 28-320 of SEQ ID NO: 5, or any combination thereof, and/or Mle046 enzyme comprising SEQ ID NO: 13 and/or a functional variant therefore having at least 80% overall sequence identity to SEQ ID NO: 13, and/or mutant Mle046 enzyme comprising SEQ ID NO: 14, or a functional variant thereof having at least 80% overall sequence identity to SEQ ID NO: 14, and wherein the MHETase-like enzyme has an enzymatic activity for degrading MHET to terephthalic acid (TPA) and ethylene glycol (EG).

20. The dual enzyme composition according to claim 19, wherein the at least one PETase-like enzyme and/or the at least one MHETase-like enzyme is linked in frame with at least one signal peptide and/or at least one purification tag.

21. The dual enzyme composition according to claim 19, wherein the molar ratio of the PETase-like enzyme to the MHETase-like enzyme is in a range from about 1:99 to about 99:1.

22. A method for producing cis-cis muconate, the method comprising contacting a substrate comprising terephthalic acid (TPA) with a recombinant bacterial cell containing a mutation in its genome, the mutation eliminating expression of muconate cycloisomerase enzymatic activity with SEQ ID NO: 4, wherein the mutation is a deletion and/or insertion of at least one nucleotide or more and wherein the substrate is produced by degradation of biomass waste and/or plastic material.

23. The method of claim 22, wherein the degradation includes an oxidative hydrothermal dissolution (OHD) process and/or reacting the substrate with the dual enzyme composition comprising at least one PETase-like enzyme and at least one MHETase-like enzyme,
wherein the PETase-like enzyme comprises at least amino acids 28-320 of the polypeptide with SEQ ID NO: 5 and/or a functional variant therefore having at least 80% overall sequence identity to amino acids 28-320 of SEQ ID NO: 5, or any combination thereof, wherein the PETase-like enzyme having an enzymatic activity for degrading bis (2-hydroxyethyl) terephthalate (BHET) to mono-(2-hydroxyethyl)terephthalic acid (MHET); and
wherein the MHETase-like enzyme comprises at least amino acids 18-613 of the polypeptide with SEQ ID NO: 6 and/or a functional variant thereof having at least 80% overall sequence identity to amino acids 28-320 of SEQ ID NO: 5, or any combination thereof, and/or Mle046 enzyme comprising SEQ ID NO: 13 and/or a functional variant therefore having at least 80% overall sequence identity to SEQ ID NO: 13, and/or mutant Mle046 enzyme comprising SEQ ID NO: 14, or a functional variant thereof having at least 80% overall sequence identity to SEQ ID NO: 14, and wherein the MHETase-like enzyme has an enzymatic activity for degrading MHET to terephthalic acid (TPA) and ethylene glycol (EG).
